(19) United States
(12) Patent Application Publication
(10) Pub. No.: US 2008/0269065 A1
(43) Pub. Date: Oct. 30, 2008

(54) Conformationally Constrained Analytical Probes

(75) Inventors: Robert Lyon, Sammamish, WA (US); John A. Zebala, Sammamish, WA (US)

Correspondence Address: Syntrix Biosystems, Inc., 215 CLAY STREET NW, SUITE B-5, AUBURN W, WA, 98001, US

(73) Assignee: Syntrix Biosystems, Inc., Auburn, WA (US)

(21) Appl. No.: 11/834,533

(22) Filed: Aug. 6, 2007

Related U.S. Application Data
(60) Provisional application No. 60/915,106, filed on Apr. 30, 2007.

(51) Int. Cl. C40B 30/04; G01N 33/00; G01N 33/53; C40B 60/12; C12Q 1/68

(52) U.S. Cl. 506/9; 436/94; 436/86; 435/7.2; 435/6; 506/39

(57) ABSTRACT

There is disclosed probes bearing partial metal chelators that in some embodiments are conformationally constrained. In certain embodiments such probes are useful in methods for the detection of a specific target molecule. These target molecules may include oligonucleotides, peptides, proteins, polysaccharides, or small molecules. There is further disclosed the use of probes with partial metal chelators engaged in a coordination complex with one another that imposes a structural constraint in the probe and increases the specificity factor of the probe.

### CROSS REFERENCE TO RELATED APPLICATION

This application claims priority to U.S. provisional patent application 60/905,106 filed on 30 Apr. 2007.

This disclosure was developed with government funding under National Institutes of Health Grant Nos. R44CA94612 and R44CA94419. The U.S. Government may have certain rights in the invention.

### 1. TECHNICAL FIELD

The present disclosure provides probes bearing partial metal chelators that in some embodiments are conformationally constrained. In certain embodiments such probes are useful in methods for the detection of a specific target molecule. Such target molecules may include, for example, oligonucleotides, peptides, proteins, polysaccharides, or small molecules. The disclosure further provides a use of probes with partial metal chelators engaged in a coordination complex with one another. This imposes a structural constraint in the probe and increases the specificity factor of the probe.

### 2. BACKGROUND

Probes which bind specifically to a particular target molecule through a binding element in the probe architecture are widely used in analytical and bioanalytical chemistry. Examples of probe binding elements used in the art include small-molecules, nucleobase polymers (e.g. DNA, RNA and PNA), peptides, peptoids and polysaccharides. Probes which further contain a label are particularly useful for the determination of a specific target molecule present in a complex sample.

### 2.1 Stemmed Molecular Beacons

Probes containing nucleobase polymers that specifically bind to a complementary target molecule (e.g. single strand or duplex DNA or RNA) may be readily designed using predictable and well-understood binding rules (e.g. Watson-Crick and Hoogsteen base pairing rules). Tyagi et al. have introduced a probe design which they call molecular beacons' (Tyagi and Kramer, Nat. Biotechnol. 14:303, 1996; Bonnet, et al., Proc. Natl. Acad. Sci. USA, 96:6171, 1999; U.S. Pat. No. 5,925,517; and U.S. Pat. No. 6,150,097). Molecular beacons employ a DNA binding element flanked on either end by two stretches of self-complementary DNA that together form a duplex stem with one another through Watson-Crick base-pairing (e.g., stem loop). The duplex stem causes the probe to form a hairpin structure, wherein the intervening binding element is presented as a looped-out region of single-stranded DNA. When the binding element contacts a complementary target molecule, the duplex stem of the molecular beacon separates as a result of forming the semi-rigid linear probe-target complex.

The conformational switch that molecular beacons undergo upon binding to the target molecule introduces a convenient means for detecting the binding event. Generally, a fluorophore is attached to one end of the molecular beacon and a fluorescence quencher is attached to the opposite end. When the two ends are in a duplex stem in the absence of the target molecule, the fluorophore is significantly quenched by the quencher since they are in close physical proximity to one another. When the two ends are separated upon formation of a probe-target complex, an increase in the signal from the fluorophore is observed and provides a convenient means for detecting binding of the target molecule.

There are several disadvantages with the design of molecular beacons. One disadvantage is the incorporation of several additional nucleobases to bind the termini of the probe together through the formation of the internal duplex stem. These additional nucleobases either alone, or in combination with the intervening binding element, may bind fortuitously to molecules in a sample other than the intended target molecule, creating the potential for false positive binding events. This possibility becomes a significant concern when the intended target molecule is part of a sample of high sequence complexity, containing potentially thousands of other nucleobase sequences other than the intended target nucleobase sequence.

Another disadvantage of using additional nucleobases to constrain the structure of the molecular beacon probe is the additional synthetic steps required in its manufacture. In a typical molecular beacon, the target binding sequence contains 15-20 nucleobases, while an additional 5 nucleobases are present on each end. Thus, 10 nucleobases out of a total of 25-30 nucleobases are extraneous to target binding. This represents a 30-40% increase in the number of manufacturing steps, each of which consumes time and chemical reagents. This becomes a significant burden when many thousands of probes are required, as might be the case if such probes were intended for use in a microarray.

An additional disadvantage of molecular beacons is that in order for the probe to perform correctly, a proper balance must be found between the binding energy of the probe-target complex and the binding energy of the internal duplex stem. For a given target sequence of interest, finding this balance is often an empirical exercise requiring the synthesis of multiple molecular beacons with different terminal sequences. This process is expensive and time consuming.

### 2.2 Stemless Molecular Beacons

Other investigators have developed “stemless” DNA, PNA and peptide beacons that dispense with a duplex stem while still constraining the conformation of the binding element in the absence of target (Gildea et al., WO A-9921881, 1999; Kuhn et al., J. Am. Chem. Soc., 124(6):1097, 2002; Wei et al., Anal. Chem., 66:1500, 1994; Packard et al., Proc. Natl. Acad. Sci. USA, 93:11640, 1996; Geoghegan et al., Bioconjugate Chem., 11:71, 2000; Wei and Herron, J. Molec. Recog., 15:311, 2002; and U.S. Pat. Nos. 6,037,137; 6,482,655; and 6,576,419). In the absence of target, stemless beacons adopt a target-free conformation mediated by a fluorophore-fluorophore association, a fluorophore-quencher association, a tertiary structure of the binding element, and/or a globular collapse of the binding element. When the binding element contacts a complementary target molecule (e.g. a nucleobase polymer, antibody or protein), the target-free conformation is abolished with the formation of the probe-target conformation. As with molecular beacons, the conformational switch that stemless beacons undergo upon binding to the target molecule provides a convenient means for detecting the binding event.

There are several disadvantages associated with stemless beacons. One disadvantage is that the target-free conformation is often unique to particular fluorophores, quenchers and binding elements, and therefore is not a general strategy for developing conformational-switching probes with uniform and predictable spectral properties. For example, while peptides end-labeled with fluorescein and tetramethylrhodamine form a dye-dye association, peptides end-labeled with fluorescein do not (Geoghegan et al., Bioconjugate Chem., 11:71, 2000). In other cases, the binding element is engineered to adopt a particular tertiary conformation, as was the case for the bent polypeptide derived from α₁-antitrypsin described by Packard et al. (Packard et al. Proc. Natl. Acad. Sci. USA, 93:11640, 1996). In examples where the binding element adopts a collapsed random coil in the absence of target (e.g. PNA), the target-free conformation and therefore baseline label output (e.g. fluorescence) will depend unpredictably on the composition (e.g. particular nucleobase sequence) of the binding element.

Another disadvantage of stemless beacons is that the target-free conformation is relatively fragile, being mediated only by weak intra-probe interactions between fluorophores, quenchers or in the binding element itself. For example, the K_{d} of the tetramethylrhodamine-tetramethylrhodamine association is estimated to be only about 0.5 mM (Geoghegan et al., Bioconjugate Chem., 11:71, 2000). Such weak interactions endow stemless beacons with low specificity factors, making them susceptible to incorrectly forming complexes with low-affinity and non-specific targets whose binding energies are sufficient to break the target-free conformation. In other cases, idiosyncratic tertiary conformations within the binding element may be more energetically favorable than the weak dye-dye association, precluding formation of the target-free conformation altogether and further limiting the general applicability of the stemless approach.

An additional disadvantage of stemless beacons that employ dye-dye associations to mediate the target-free conformation is that the dye both constrains and labels the probe. It can be problematic to optimize either the label or constraint without affecting aspects of the other. Thus, the inability to independently adjust the energetics of the target-free conformation and the spectral output of the probe is a major disadvanatage of the stemless approaches.

### 2.3 Probes Bearing ‘Partial Metal Chelators’ (See “5.1 Glossary”)

A fluorophore with a partial metal chelator and adjacent alcohol has been attached to a terminal end of a peptide probe that is a substrate for a kinase (Chen et al., Biochemica et Biophysica Acta, 1697:39, 2004 and Chen et al., J. Am. Chem. Soc., 124:3840, 2002). Phosphorylation of the alcohol by the kinase caused the partial metal chelator to complex with the added phosphoryl moiety in the presence of a metal cation. The complexation with the metal cation caused a phosphorylation-induced fluorescence change in the fluorophore.

A partial metal chelator may be introduced into an analytical probe to allow the probe, either bound to its target or in isolation, to be ‘captured’ onto a solid support that displays metal ions (U.S. Pat. No. 6,346,378; and U.S. Pat. App. 20030207296).

A partial metal chelator has been incorporated into an oligonucleotide probe to increase the stability of the probe-target duplex (Mokhir et al., Bioorg. Med. Chem. Let. 13:1399, 2003; Mokhir et al., J. Am. Chem. Soc., 126:6208, 2004). In the duplex state, the metal atom remained partially bound to the probe metal binding site, but also coordinated with elements of the target.

Therefore, the art teaches the use of partial metal chelators in probe labeling, probe capture, and probe-target stabilization, wherein the number of partial metal chelators is strictly limited to one per probe.

Probes bearing two partial metal chelators derived from terpyridine have been incorporated in DNA (Goritz and Kramer, J. Am. Chem. Soc., 127:18016, 2005). However, these authors reported that the metal-cyclized probe was unable to bind target DNA molecules, and that addition of metal to preformed probe-target complexes actually resulted in dissociation from their targets.

### 2.4 Metal Chelates As Structural Elements in Probes

Zinc finger peptides have been synthesized and studied with regard to conformational stablization and peptide folding as a function of zinc binding. However, although zinc finger peptidesare not able to bind DNA in a manner similar to the transcription proteins from which they are derived (Krizek et al., J. Am. Chem. Soc., 113:4518, 1991 and Rhodes et al., Sci. Am., 268(2):56, 1993).

Stabilization of alpha helix in short peptides has been reported by making an exchange-inert ruthenium^{III} complex (Ghadiri and Femholz, J. Am. Chem. Soc., 112:9633, 1990) or an exchange-labile Cu, Zn, or Cd complex (Ghadiri and Choi, J. Am. Chem. Soc. 112:1630, 1990) with peptides that have a propensity to form helical structures. In 17 amino acid-long peptides, two His residues or a Cys and a His residue were placed at i and i+4 positions which would reside on the same side of two consecutive turns in an α-helix and formed an exchange-inert complex with cis[Ru(III)(NH₃)₄(H₂O)₂]²⁺ or an exchange-labile complex with Zn, Cu, or Cd. The resulting complexes were shown by circular dichroism studies to have a higher helical content (U.S. Pat. Nos. 5,200,504; 5,408,036; and 5,410,020). The polypeptide itself serves only in a structural capacity with no affinity for a target.

Cyclic and linear peptides contain a biological-function domain that can bind a target, and a plurality of amino acids that provide a metal ion-binding backbone that complex a metal ion (U.S. Pat. Nos. 5,891,418; 6,027,711; and 6,331,285). Binding by metal conformationally constrains the biological-function domain. The characteristics of metallopeptides are that a) target binding does not disrupt the metal complex, b) the cyclic peptides are connected entirely via covalent linkages and thus cannot be linearized upon binding to target, and c) the affinity for target is generally substantially higher when the metal ion-binding backbone is complexed with the metal ion than the affinity for target when the metal ion-backbone is not complexed with the metal ion.

Two partial metal chelators of γ-carboxyl-glutamic acid have been suggested to cyclize a peptide (U.S. Pat. Nos. 5,510,240; 5,650,489; 5,855,882; 5,858,670; 5,861,238; 5,888,763; 5,891,341; 5,962,245; 6,030,780; 6,077,682; 6,162,627; 6,312,887; and 6,495,139). Cyclization through chelation achieved conformational constraint. The binding of a probe to a target resulted in no disruption of the chelate. A peptide fragment related to a natural calcium binding protein exhibited enhanced α-helical structure upon binding to calcium (Shaw et al., Science 249:280, 1990; and Reid et al., J. Biol. Chem., 256:2742, 1981). This was due to dimerization of two helical peptide segments located at each end, which was induced by complexation of a calcium ion in the middle peptide segment.

### 3. SUMMARY

The present disclosure provides analytical probes comprises an element which possesses binding affinity for a target analyte, and two partial metal chelators. In the presence of a transition metal and in the absence of a target, the two partial metal chelators together form a coordination complex with a single metal ion. Binding of the probes to their targets produces a conformational change in the probes, forcing the two partial metal chelators apart and disrupting the coordination complex. Certain embodiments of probes employ interactive labels, which allow the conformational change to be detected.

In a preferred embodiment, a probe is provided comprising first and second partial metal chelators attached to one another through a portion of a flexible binding element, wherein a specificity factor of the probe is enhanced in the presence of a transition metal. In these embodiments, the specificity factor preferably comprises a value selected from the group consisting of K_{d}, IC₅₀, Kₘ, kₐ, k_{d}, log₂(PM/MM), Tₘ, T_{d-50} and T_{d-w}.

In some embodiments, the flexible binding element comprises a segment selected from the group consisting of PNA, poly-morphilino, PNAMs, DNA, RNA, siRNA, peptide, and oligosaccharide. In other embodiments, the first and second partial metal chelators may selected from the group consisting of:

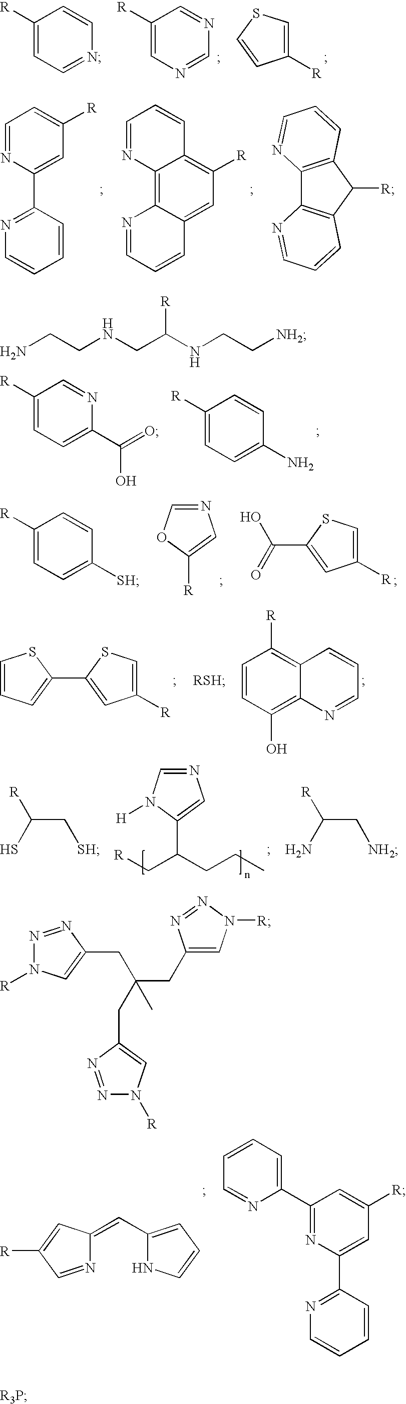

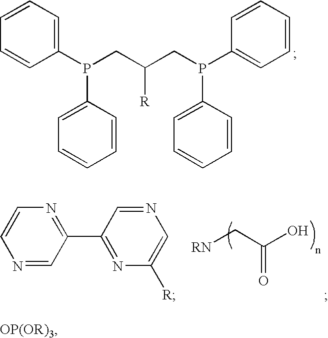

where R is the attachment to the probe.

In a preferred embodiment, a plurality of probes according to the disclosure are attached to a microarray. In other preferred embodiments, the first and second partial metal chelators are the same.

In some embodiments, the transition metal is selected from the group consisting of zinc, cadmium, copper, nickel, ruthenium, platinum, palladium, cobalt, magnesium, barium, strontium, iron, vanadium, chromium, manganese, rhodium, silver, mercury, molybdenum, tungsten, calcium, lead, cerium, aluminum and thorium.

Also provided is a method of binding a target comprising (a) providing a probe according to the disclosure; and (b) contacting the probe with the target and a transition metal. In preferred embodiments, a specificity factor of the probe is enhanced in the presence of a transition metal and the target. In preferred embodiments, the method of binding a target occurs inside a cell, and more preferably alters (e.g. reduces) the amount of mRNA or protein in the cell.

Also provided is a method of binding a target comprising (a) providing a probe comprising first and second partial metal chelators covalently attached to one another through a portion of a flexible binding element, wherein the partial metal chelators form a coordination complex with a transition metal, and wherein the coordination complex is disrupted when the binding element contacts a target; and (b) contacting the probe with the target and a transition metal.

Other embodiments of the disclosure provide a partial metal chelator synthon of structure

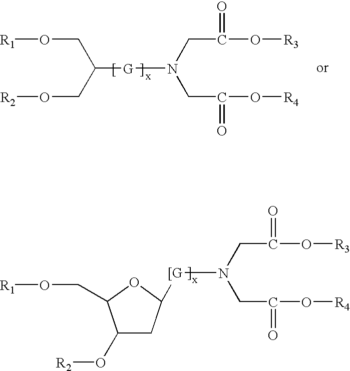

wherein
R₁ is a hydroxyl protecting group;
R₂ is selected from the group consisting of a linker;

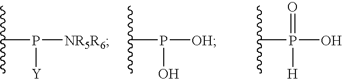

and salts thereof;
R₃ and R₄ are carboxyl protecting groups;
R₅ and R₆ are independently selected from the group consisting of C₃₋₁₀ branched alkyl and C₁₋₁₂ unbranched alkyl, and cyclic hydrocarbons;
Y is beta-cyanoethyl;
G is selected from the group consisting of alkyl, heteroalkyl, aryl, aryl(alkylene), heteroaryl, heteroaryl(alkylene), carbocycle, carbocyle(alkylene), heterocycle, heterocycle(alkylene),

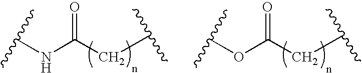

wherein n=1 to 10; and
X is from 0 to 10.

The partial metal chelator synthon may further be attached to controlled pore glass beads or flat glass. A preferred partial metal chelator synthon is a compound with structure

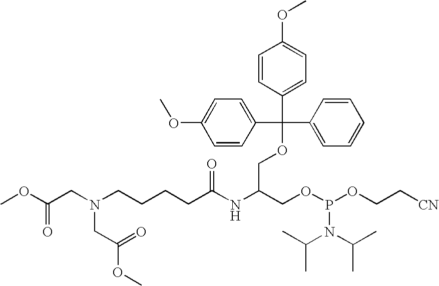

### 4. BRIEF DESCRIPTION OF THE DRAWINGS

FIG. 1A is a diagram of a binding element and two partial metal chelators with at least part of the binding element lying between them.

FIG. 1B is a diagram of ‘closed state’ of the probe where, in the absence of target and in the presence of a transition metal, the two partial metal chelators together form a coordination complex with a single metal ion.

FIG. 1C is a diagram of the ‘open state’ of the probe where binding of the probe to a target molecule produces a conformational change in the probe which forces the two partial metal chelators apart and disrupts the coordination complex.

FIG. 2 is a diagram of melting curves of a probe with its DNA target and with a DNA strand containing a single base mismatch in the middle of the sequence, as prepared in Example 6.2.2.

FIG. 3 is a diagram of melting curves of a dihistidine probe with varying concentrations of nickel as prepared in Example 6.2.3.

FIG. 4 is a diagram of the melting temperature versus nickel concentration for dihistidine and hexahistidine probes.

FIG. 5 is a diagram of the fluorescence exhibited by a probe with a fluorophore/quencher pair in the open and closed conformations as provided in Example 6.3.4.

FIG. 6 is a diagram of fluorescence spectra of a probe provided in Example 6.3.6.

FIG. 7 is a diagram of the kinetic binding curves of a probe in the presence and absence of a transition metal as provided in Example 6.2.6.

### 5. DETAILED DESCRIPTION

### 5.1 Glossary of Terms Used

“Affinity” means a binding interaction between two molecules. Affinity can be measured and quantitated indirectly in an assay. Assays suitable for measuring affinity will be known to those skilled in the art, and include, but are not limited to assays that measure spectral absorption or transmission (e.g. chromogenic changes or hyperchromic shifts in the UV, visible, and IR portions of the spectrum), fluorescence, fluorescence resonance energy transfer (FRET), fluorescence polarization, excimer formation, phosphorescence, luminescence (i.e. chemiluminescence), catalytic activity (e.g. via a chromogenic substrate), molecular weight (e.g. using quantitative MALDI-TOF, ESI-MS, LC-MS), charge, density, melting point, chromatographic mobility (e.g. LC-MS, affinity column chromatography), electrophoretic mobility (e.g. polyacrylamide or agarose gel-shift assays), turbidity (e.g. nephelometry), diffraction, nuclear magnetic resonance (e.g. ³H-NMR), surface plasmon resonance, elemental composition, structure (e.g. co-crystal structures using x-ray diffraction cystallography), binding by an antibody (e.g. by ELISA), radioactivity (e.g. quantitative autoradiography in a solid-phase capture assay), nucleic acid quantity (e.g. using real time PCR or colony counting after in vivo transfection), and in vivo dose response (e.g. in an animal xenograft model or in a human). Numerical values are often obtained from assays that are useful for quantitating affinity. These include, but are not limited to the thermal-dissociation temperature of a nucleobase polymer homo- or heteroduplex (Tₘ), the dissociation constant (K_{d}), the 50% inhibitory concentration (IC₅₀), the Michaelis-Menten constant (Kₘ), and the inhibitory constant (K_{I}). As used in this disclosure, relative differences in such numerical values are used to express relative differences in affinity (see “specificity factor” below).

“Alkyl” means a saturated or unsaturated; unsubstituted or substituted (substituted by, for example, Cl, Br, F, I, —NH₂, —OH, ═O, —NO₂, —COOH, —SO₃H, —SO₂NH₂, —CF₃, or C₆₋₂₀ aryl); straight chain, branched chain, or cyclic hydrocarbon moiety having 1 to 20 carbon atoms and preferably from 1 to 6 carbon atoms this chain or cyclic hydrocarbon moiety may be interrupted by at least one heteroatom such as N, O or S.

“Aryl” means a carbocyclic moiety which may be substituted by, for example, Cl, Br, F, I, —NH₂, —OH, ═O, —NO₂, —COOH, —SO₃H, —SO₂NH₂, —SO₂(alkyl), —CF₃, or C₆₋₂₀ aryl; and containing one or more benzenoid-type rings preferably containing from 6 to 40 carbon atoms, this carbocyclic moiety may be interrupted by at least one heteroatom such as N, O or S.

“Aralkyl” means an aryl group attached to the adjacent atom by an alkyl group (e.g., benzyl), preferably containing from 6 to 30 carbon atoms.

“Alkoxyalkyl” means a substituted or unsubstituted alkyl group containing from 1 to 30 carbon atoms and preferably from 1 to 6 carbon atoms, wherein the alkyl group is covalently bonded to an adjacent element through an oxygen atom (e.g., methoxy and ethoxy).

“Complete metal chelator” refers to a chelating moiety with a plurality of ligands that coordinate with a transition metal, wherein the plurality of ligands equals or exceeds the maximum number of ligands the transition metal can except, and all of the plurality of ligands are connected to one another by one or more chemical bonds that are devoid of a binding element (see also “partial metal chelator”). Thus, a transition metal cannot be simultaneously bound by more than one complete metal chelator (e.g. EDTA). Whether a particular chelating moiety is a partial or complete metal chelator will depend on the metal, and in some cases a particular chelating moiety will be a partial metal chelator with one metal and a complete metal chelator with another metal.

“Inhibition” refers to contacting an enzyme or receptor with a probe of the invention (i.e. the “inhibitor”) such that the probe interferes with the ability of the enzyme or receptor to engage in enzymatic catalysis, binding with another protein, or both. A probe is said to cause “inhibition”, and thus “inhibit”, if the probe can be contacted with the enzyme or receptor in a sufficient concentration to reduce enzymatic catalysis, binding with another protein, or both, by at least 50% as measured in an assay. In some embodiments, inhibition may be associated with covalent modification of the inhibitor although this is not a requirement of the invention. In preferred embodiments, an inhibitor is not covalently modified during inhibition.

An “interactive label pair” includes a pair of labels wherein at least one label exhibits a measurable characteristic upon binding of the probe to a target molecule. An interactive label pair can be selected from FRET and non-FRET pairs. Common labels in the context of the present invention include: the dye fluorescein, and fluorescein derivatives, such as 5-carboxyfluorescein (5-FAM), 6-caroxyfluorescein (6-FAM), 2′7′-390 dimethoxy-4′5′-dichloro-6-carboxyfluorescein (JOE), hexachloro fluorescein (HEX), tetrachloro fluorescein (TET); rhodamine, and rhodamine derivatives, such as N,N,N′,N′-tetramethyl-6-carboxyrhodamine (TAMRA), 6-carboxyrhodamine (R6G), tetramethyl-indocarbocyanine (Cy3), tetramethyl-benzindocarbocyanine (Cy3.5), tetramethyl-indodicarbocyanine (Cy5), tetramethyl-indotricarbocyanine (Cy7), 6-carboxy-X-rhodamine (ROX); R-phycoerythrin; 4-(4′-dimethylaminophenylazo) benzoic acid (DABCYL); 5-(2′-aminoethyl)aminonaphthalene-1-sulfonic acid (EDANS); tetramethylrhodamine (TMR); pyrene; 3-(ε-carboxy-pentyl)-3′-ethyl-5,5′-dimethyloxacarbocyanine (CYA); Erythrosine; the BODIPY series; the AlexaFluor series; Oregon Green; Iowa Black; Black Hole Quenchers (1 & 2); Eosine; Lucifer Yellow; Texas Red; 4-dimethylaminophenylazophenyl-4′-maleimide (DABMI); Malachite Green; Coumarin, and coumarin derivatives; dimethylaminoazosulfonic acid (DABSYL); and Methyl Red.

A “label” is a moiety that facilitates detection of a target molecule. Common labels in the context of the present disclosure include fluorophores and quenchers. For example, one or more atoms within the compound may be replaced with radioactive isotopes. Alternatively, labels may provide antigenic determinants, radioactive isotopes, non-radioactive isotopes, nucleic acids available for hybridization, altered fluorescence-polarization or altered light-scattering. Still other labels include those that are chromogenic, chemiluminescent or electrochemically detectable. Other methods available to label a probe will be readily apparent to those skilled in the art, and as discussed more fully below.

A “ligand” refers to a site on either a partial or complete metal chelator with an unshared electron pair that is capable of forming a coordination bond with a metal ion (see also “partial metal chelator” and “complete metal chelator” definitions). For example, the common chelator ethylenediamine tetraacetic acid (EDTA) possesses six ligands—two amino nitrogen atoms and four carboxylate functionalities—and is thus called a hexadentate ligand. Different metal ions are capable of coordinating with a different number of ligands. Many transition metals can accept a maximum of six ligands and thus adopt octahedral coordination geometry. For these metals, EDTA represents a complete metal chelator, since the metal atom would be completely bound by the EDTA group and has no additional ligand sites available for coordination.

A “linker” is a molecule that connects and spaces a probe or other molecule from a solid-support. Linkers may further facilitate target molecule binding by the probe, or may supply a labile linkage that allows probes to be detached from the solid-support (e.g. a photocleavable linkage). The composition of a linker in preferred embodiments is as described for the composition of a spacer (see ‘spacer’ in “5.1 Glossary”).

A “nucleobase” is a nitrogenous heterocyclic group typically found in nucleic acids (such as the purine bases adenine and guanine, or the pyrimidine bases cytosine, thymine and uracil), or an analog of such a group. Analogs include, for example, purine bases in which the ring substituents are other than those found in adenine or guanine, or pyrimidine bases in which the ring substituents are other than those found in uracil, thymine and cytosine. A number of analogs of nucleobases are well known in the art; many of which have been tested as chemotherapeutic agents. Some of these are described herein; see also, e.g., Beilstein's Handbuch der Organischen Chemie (Springer Verlag, Berlin), and Chemical Abstracts, which provide references to publications describing the properties and preparation of such compounds.

A “nucleobase polymer” is a polymer of nucleobases linked to a backbone. The backbone may be naturally occurring (as in a nucleic acid molecule) or may be non-naturally-occurring. Nucleobase polymers with non-naturally-occurring backbones are preferably resistant to degradative enzymes. Representative examples include peptide nucleic acids (see Buchardt et al., PCT WO 92/20702 and Buchardt et al., U.S. Pat. No. 5,719,262), morpholino-based nucleobase polymers (see Summerton and Weller, U.S. Pat. No. 5,698,685; Summerton et al., U.S. Pat. No. 5,378,841 and Summerton and Weller, U.S. Pat. No. 5,185,444), peptide-base nucleic acid mimics or PENAMs (see Shah et al., U.S. Pat. No. 5,698,685), and polynucleosides with linkages comprising carbamate (see Stirchak and Summerton, J. Org. Chem. 52:4202, 1987), amide (see Lebreton et al., Synlett. February 1994:137), methylhydroxylamine (see Vasseur et al., J. Am. Chem. Soc. 114:4006, 1992), 3′-thioformacetal (see Jones et al., J. Org. Chem. 58:2983, 1993), sulfamate (see Huie and Trainor, U.S. Pat. No. 5,470,967) and others (see Swaminathan et al., U.S. Pat. No. 5,817,781 and Freier and Altmann, Nucl. Acids Res. 25:4429, 1997, and references cited therein).

“Partial metal chelator” refers to a moiety with a plurality of ligands that coordinate with a transition metal, wherein all of the plurality of ligands are less in 460 number than the maximum number of ligands the transition metal can except, and all of the plurality of ligands are connected to one another by one or more chemical bonds that are devoid of the binding element (see also “complete metal chelator”). Whether a particular chelating moiety is a partial or complete metal chelator will depend on the metal, and in some cases a particular chelating moiety will be a partial metal chelator with one metal and a complete metal chelator with another metal. In the probes disclosed herein, the internal structural constraint mediated by the coordination complex is achieved through complexation with a transition metal. Thus, the probe can only be constrained by two partial metal chelators that ‘share’ the transition metal between them. Accordingly, the number of ligands in one partial metal chelator must be less the maximum number of ligands the transition metal can except (i.e. coordinate). As a contrasting example that would not be an embodiment of the present disclosure, a probe bearing two complete metal chelators (e.g. EDTA) each supplying a maximum number of ligands to a transition metal would result only in the open probe conformation with each complete metal chelator independently binding a single transition metal (i.e. more than one complete metal chelator cannot simultaneously bind a single transition metal ion). Partial metal chelators are designated as bidentate, tridentate and tetradentate ligands depending on whether they provide two, three, and four ligands, respectively.

A “peptide nucleic acid” (PNA) is a molecule comprising repeating units of N-(2-aminoethyl)-glycine linked by amide bonds (see Buchardt et al., PCT WO 92/20702). Unlike the natural DNA backbone, no deoxyribose or phosphate groups are present. The bases are attached to the backbone by methylene carbonyl linkages.

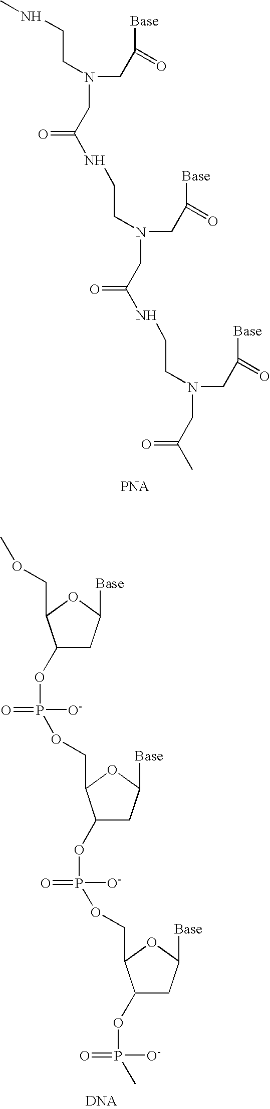

In this disclosure, PNA sequences are written using the single-letter designation of the attached base just as DNA sequences are written. PNA sequences are distinguished from DNA sequences by an “NH₂” group at what would be the 5′ end of a DNA sequence. For example, in this disclosure AGGTC-5′ is a DNA sequence, while AGGTC-NH₂ is a PNA sequence. If the “NH₂” is not designated in a particular PNA or otherwise peptide-linked probe (e.g. see the Examples), the convention used herein will be that the probe is written left to right from the carboxy to the amino termini, respectively. Similarly, if the “5′” is not designated in a particular DNA probe, it will be understood that the convention used herein will be that the probe is written left to right from the 5′ to the 3′ termini, respectively. Certain preferred peptide nucleic acid polymers comprise a repeating unit of the form:

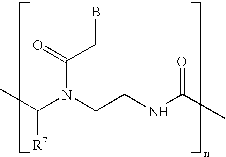

wherein each B is independently selected from the group consisting of nucleobases; each R⁷ is independently selected from the group consisting of hydrogen, C₁-C₈ alkylamines and spacers; and each n is an independently selected integer ranging from 1 to 100.

A “peptide nucleic acid mimic” (PENAM) is a nucleobase polymer that comprises a repeating unit of the form:

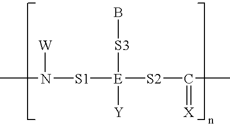

wherein each is E is independently selected from the group consisting of carbon and nitrogen; each W is independently selected from the group consisting of hydrogen and spacers; each Y is independently selected from the group consisting of hydrogen and spacers, in repeating units wherein E is carbon; each Y is a lone pair of electrons, in repeating units wherein E is nitrogen; each S1 is optional, and if present is an independently selected first spacer; each S2 is optional, and if present is an independently selected second spacer; each S3 is optional, and if present is an independently selected third spacer; each X is independently selected from the group consisting of oxygen and sulfur; each B is independently selected from the group consisting of nucleobases; N is nitrogen; and each n is an independently selected integer ranging from 1 to 100.

A “spacer” is a molecule that connects and separates probe components from one another (e.g. separates the binding element from a chelating chelating group). A spacer may sometimes be referred to as a “spacer group.” A spacer is relatively small, containing a backbone of 1-30 atoms (not counting hydrogen atoms), preferably selected from carbon, nitrogen, oxygen, and sulfur. Typically, such spacers comprise substituted or unsubstituted alkyl, alkenyl, alkenyl groups or aminoalkyl carboxylic acids, the side chain of an amino acid, natural and unnatural amino acids, aminooxyalkylacids, alkyl diacids, alkyloxy diacids, alkyldiamines, and FMOC-AEEA (Applied Biosystems, Foster City, Calif.). However, spacers can also comprise for example: carbonyl(C═O), thiocarbonyl(C═S), amine (NH), substituted amine (NR), amide(C(═O)NH), substituted amide(C(═O)NR), carbamate (NHC(═O)O), urea (NHC(═O)NH), thioamide(C(═S)NH), substituted thioamide(C(═S)NR), hydrazine (NH—NH), substituted hydrazine (N(R)—N(R)), ether (C—O—C), thioether (C—S—C), disulfide (S—S), sulphone (S(═O)) and/or sulphoxide (SO₂) groups. A spacer can also be substituted with one or more small chemical groups, for example, small chain alk(ane, ene, yne)s, hydroxyl, alkoxyl, ketone, aldehyde, thiol, amino and/or halogen groups. In some embodiments a spacer may be photocleavable.

The “specificity factor” refers to the degree to which the affinity of a probe for a target molecule differs from the affinity of the probe for another, typically structurally related molecule. The specificity factor also refers to the amount by which the affinity of a free binding element for a target molecule differs from the affinity of the binding 540 element for another, typically structurally related molecule. The specificity factor may be used to characterize the probe or binding element in the open or closed state, or in the presence or absence of metal, or any combination thereof (the context of the description will determine which species is being described). In addition to this qualitative definition, the specificity factor may be defined as a numerical ratio or numerical difference in affinity values that are quantified in an assay. For example, the specificity factor (SF) of a probe or binding element for a particular target molecule relative to another structurally related molecule, may be described as the ratio of their equilibrium dissociation constants (e.g. non-target K_{d}/target K_{d}), the ratio of their 50% inhibitory concentrations (e.g. non-target IC₅₀/target IC₅₀), the ratio of their Michaelis-Menten constants (e.g. non-target Kₘ/target Kₘ), or the ratio of their inhibition constants (e.g. non-target K_{I}/target K_{I}). The ratio of non-equilibrium association (kₐ) or dissociation (k_{d}) constants are also suitable specificity factors. When the ratio of physical constants is determined in the presence of metal it is denoted SF_{+M}, and when it is determined in the absence of metal it is denoted SF_{-M}. In a further example where the binding element is a nucleobase polymer, the specificity factor of a probe or binding element for a particular target molecule relative to another structurally related molecule (e.g. a molecule identical to the target molecule except for a single nucleobase change), may be described as the difference in their thermal-dissociation temperatures (ΔTₘ=target Tₘ−non-target Tₘ). When the ΔTₘ is determined in the presence of metal it is denoted ΔT_{m+M}, and when it is determined in the absence of metal it is denoted ΔT_{m-M}. In the above examples, increasing values of the specificity factor correspond to an increasing preference for the probe to bind the target molecule over another, typically structurally related molecule. Suitable specificity factors in microarray applications include the difference between perfectly complementary and single-base mismatch values for T_{d-50} or T_{d-w} (for definitions of these non-equilbrium values, see Wick et al., Nucleic Acids Res., 34:e26, 2006). A preferred specificity factor in microarray embodiments is log₂(PM/MM), where PM is the fluorescence intensity of a perfectly matched probe and MM is the fluorescence intensity of a probe with a single mismatch (Wick et al., Nucleic Acids Res., 34:e26, 2006). A preferred specificity factor in RNAi applications is the ratio of on-target to off-570 target reduction in RNA or protein levels (Jackson et al., RNA, 12:1197, 2006). A specificity factor is said to be ‘enhanced’ if the relative affinity to a desired target compared to a structurally related molecule increases or improves. In common usage in the art, if a first probe had an enhanced specificity factor relative to a second probe, it would be said ‘that the first probe is more specific than the second probe’. It is desirable, although not required, to express a specificity factor in such a way that it numerically increases when the specificity factor is enhanced. For example, a specificity factor that is enhance may in fact numerically decrease.

A “target molecule” or “target” is a molecule for which a probe as affinity, and is 580 able to bind. Target molecules may be naturally-occurring or man-made molecules, and can be employed in their unaltered state or as aggregates with other species. Target molecules may covalently or non-covalently modify a given probe after binding by the probe, and vice versa. Such modifications include, but are not limited to labeling, altering conformation, cleaving (e.g. caspase, nuclease), and covalently binding. Examples of target molecules include, but are not limited to, antibodies, monoclonal antibodies, cell membrane receptors (e.g. kinases), enzymes, proteases (e.g. caspases), nucleases, drugs, polynucleotides, nucleic acid, aptamers, catalytic nucleic acids, peptides, catalytic peptides, PNA, morpholino-based nucleobase polymers, other nucleobase polymers, cofactors, lectins, sugars, polysaccharides, cells, cellular membranes and organelles.

“Tₘ” refers to the equilibrium temperature at which a nucleobase-duplex (e.g., DNA:DNA, PNA:DNA or PNA:PNA) dissociates into separate nucleobase polymers such that the combined concentration of separate nucleobase polymers is twice the concentration of the intact nucleobase-duplex. In an alternative definition, the Tₘ refers to the temperature at which the first derivative of the curve plotting light absorbance of a nucleobase-duplex, usually at about 260 nm, versus temperature is at a maximum. Depending on the nucleobase-duplex, the melting temperature can be influenced by pH, and salt concentration. When the Tₘ is determined in the presence of metal it is denoted T_{m+M}, and when it is determined in the absence of metal it is denoted T_{m-M}.

### 5.2 Probe Architecture and Properties

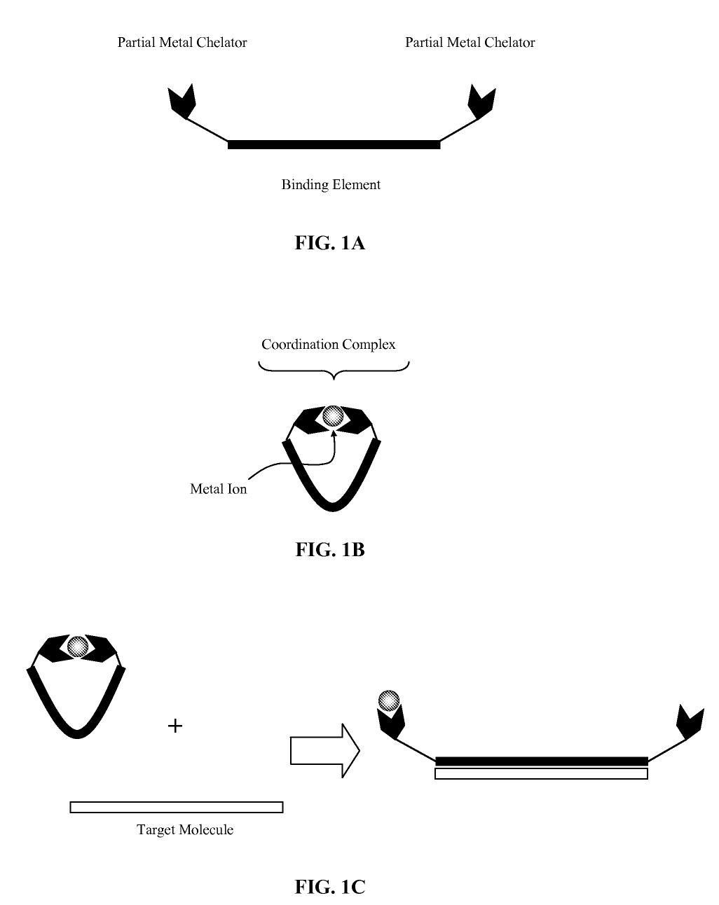

Probes comprise a binding element that possesses binding affinity for a target molecule, and two partial metal chelators (see “5.1 Glossary”) with at least part of the binding element lying between them (see FIG. 1A). In the presence of a transition metal and in the absence of target, the two partial metal chelators together form a coordination complex with a single metal ion (i.e. the ‘closed’ state, see FIG. 1B). Binding of the probe to a target molecule produces a conformational change in the probe, which forces the two partial metal chelators apart and disrupts the coordination complex (i.e. the ‘open’ state, see FIG. 1C). Although the probe is flexible while undergoing the ‘switch’ between the open and closed conformations, it cannot adopt a closed state while bound by target. Thus, there is a competition between the closed metal-bound conformation and the open target-bound conformation.

The location of the two partial metal chelators within the overall probe architecture is important to the proper functioning of the probe. There are two general requirements. First, the geometry and flexibility of the probe allows the two partial metal chelators to be close enough together that they can simultaneously coordinate a single metal ion. Second, when in the target-bound state, the two partial metal chelators are far enough apart that they cannot coordinate simultaneously to a single metal ion. Typically, the partial metal chelators are on opposing ends of the probe. This arrangement, however, is not a requirement. In some applications, it may be desirable to insert one or both of the partial metal chelators within the binding element, as described more fully below (see section “5.8 Methods of Use And Other Embodiments”).

Certain probe embodiments achieve the primary benefits of molecular beacons while avoiding many of the disadvantages. In particular, probes are constrained by the binding energy of a transition metal complex, rather than by nucleobase pairing. Thus, there are no extraneous nucleobases that may inadvertently interact with incorrect targets. The two partial metal chelators can be incorporated into the probe in as few as two synthetic steps. Substantial savings in synthesis time and material can therefore be achieved relative to molecular beacons. By using different transition metals, the ‘strength’ of the intramolecular constraint can be adjusted without any additional synthesis. This represents a significant improvement over molecular beacons, where many probes with different duplex sequences must frequently be synthesized and purified to find one with the proper constraint energy. Also, the physical size of the partial metal chelators and the resultant coordination complex is substantially smaller than the duplex stem in molecular beacons. Small target molecules that were of insufficient linear length to disrupt a molecular beacon stem upon forming the rigid target-probe complex may thus be able to disrupt the much smaller coordination complex in the probes of the disclosure.

The probe arrangement also avoids many of the disadvantages of stemless beacons as well. In particular, the use of partial metal chelators provides a general strategy for introducing conformational constraint into probes that does not rely on designing around idiosyncratic properties of particular fluorophores, quenchers and binding elements. Further, the coordination complex places the binding element and its labels in a substantially fixed and reproducible spatial orientation, providing for more uniform spectral properties among probes bearing different binding elements but having the same labels. By separating probe constraint from probe labeling, which are both mediated by the same dye molecules in certain stemless approaches, the energetics of probe-target binding and the spectral output of the probe can be independently adjusted and optimized. Finally, in contrast to the relatively fragile nature of the target-free conformation in stemless beacons, a metal coordination complex provides a strong internal constraint in the probe that markedly enhances the specificity factor of the probe, and overcomes the majority of tertiary structures that may be encountered in a binding element.

The probes have, affinity for the target molecule in the open state, and optionally, affinity for the target molecule in the closed state. Preferably, the probe in the presence of metal has an affinity for a target molecule that is characterized by a K_{d}, IC₅₀, Kₘ, or K_{I} whose numerical value is less than 1000 μM, 100 μM, 10 μM, and most preferably less than 1 μM. In embodiments where the probe is a nucleobase polymer, the probe will have an affinity in the presence of metal that is characterized by a T_{m+M} whose numerical value is between 20° C. and 100° C., between 30° C. and 90° C., between 40° C. and 80° C., and most preferably between 50° C. and 70° C.

In addition to affinity for a target molecule, the probe has specificity for the target molecule. As used herein, a probe is said to have specificity for a target molecule if it has detectably greater affinity for the target molecule than for at least one other molecule (typically one that is structurally related to the target molecule). The degree to which the affinity of the probe for a target molecule is greater than the affinity of the probe for another molecule is defined herein by a ‘specificity factor’ (see “5.1 Glossary”).

The specificity factor of the probes, described herein, is enhanced in the presence of a transition metal relative to the specificity factor of the probe in the absence of metal, or relative to the specificity factor of the free binding element. This enhanced specificity of target molecule binding is a major advantage, especially when probes are used to detect target molecules in a mixture containing a large number of molecules closely related in structure to the target molecule (e.g. expression profiling of large mRNA mixtures on arrays of probes having a nucleobase polymer binding element, or detecting one SH2 domain in a large mixture of SH2 domains using arrays of probes having a peptide binding element).

In preferred embodiments where the specificity factor (SF) is characterized by a ratio of physical constants, the specificity factor of the probe in the presence of a transition metal (SF_{+M}) is increased relative to the probe (or free binding element) in the absence of metal (SF_{-M}) by at least a factor (ASF) of 1.2, 2.0, 5.0, and most preferably by at least a factor of 20 (i.e. SF_{+M}=Δ_{SF}×SF_{-M}). In other preferred embodiments employing probes comprising nucleobase polymers where the specificity factor is characterized by a ΔTₘ, the specificity factor of the probe in the presence of a transition metal (ΔT_{m+M}) is increased relative to the probe (or free binding element) in the absence of metal (ΔT_{m-M}) by at least an amount (ΔΔTₘ) of 0.5° C., 2.0° C., 4.0° C., and most preferably by greater than 10.0° C. (i.e. ΔΔTₘ=ΔT_{m+M}−ΔT_{m-M}).

In preferred embodiments, the specificity factor of the probe in the presence of metal (SF_{+M}) is greater than 1, 2, 10, and most preferably greater than 100, where the specificity factor is characterized by, for example, a ratio of non-target K_{d}/target K_{d}, non-target IC₅₀/target IC₅₀, non-target Kₘ/target Kₘ, or non-target K_{I}/target K_{I}. In embodiments where the binding element of the probe is a nucelobase polymer, the specificity factor of the probe in the presence of metal (ΔT_{m+M}) is greater than 2° C., 5° C., 10° C., and most preferably greater than 20° C. It will be understood however, that depending on how the specificity factor is numerically constructed, that an enhancement of the specificity factor may in fact be associated with a numerical decrease in its value (e.g. ratios that are the inverse of those described above).

In preferred embodiments, the probe will exhibit the above metal-enhanced specificity factors when the structural difference between the target molecule and another molecule is only a single nucleobase, a single amino acid, or a single carbohydrate. In other preferred embodiments, the probe will exhibit the above metal-enhanced specificity factors for a target-related molecule whose structure is greater than 85%, 90%, 95%, and greater than 99% identical to the target molecule. In particularly preferred embodiments, the probe will exhibit the above metal-enhanced specificity factors for a target-related molecule that differs from the target molecule by only a single hydrogen bond donor or acceptor.

Without being bound by theory, it is believed that the metal-enhanced specificity (i.e. increased specificity factor) of these probes arises from the fact that probe binding to a target molecule must overcome the favorable energetics of the probe's internal structural constraint mediated by the metal chelate in the closed state. Molecules which are similar to the desired target molecule, but which are slightly different (and therefore exhibit slightly less favorable binding energies), may not be able to overcome this internal constraint. In these cases, the internal conformational constraint essentially ‘wins’ the competition, thereby shifting probe binding away from false-positive binding by molecules whose structure is similar, but not identical to the structure of the target molecule.

The probe is designed to have an appropriate balance between the binding energy of the target molecule and the binding energy of the conformational constraint imposed by the coordination complex. In preferred embodiments, the energy of binding to the target molecule should at least approximate or exceed the binding energy of the metal-mediated conformational constraint, allowing the probe to favor formation of the open state relative to the internal coordination complex. In less preferred embodiments, the energy of binding of the coordination complex exceeds the energy of binding to the target molecule such that the open complex cannot form, or cannot form in a reasonable period of time under a particular set of binding conditions (i.e. temperature and concentration). To be of practical use, at least 50% of the probe should form the open complex (target in excess to probe) in less than 24 hours, less than 12 hours, less than 6 hours, and most preferably less than 2 hours, under particular conditions of temperature and probe/target concentration (i.e. the rate of open complex formation).

In embodiments where the precise numerical values of these binding energies cannot be calculated precisely a priori, a suitable balance between the binding energy of the target molecule and the coordination complex can be identified by synthesizing a library of probes having differing binding elements with a range of predicted binding energies for the target molecule and then systematically testing their affinity and specificity. The library of probes may further incorporate different partial metal chelators presenting, for example, a range of from 2 to 6 ligands, in order to examine a range of binding energies of the coordination complex. Suitable probes resulting from testing will be identified as those that possess affinities and rates of open complex formation whose values are as defined above.

In still another approach to optimizing the balance between the binding energy of the target molecule and the coordination complex, different transition metals can be employed to vary the ‘strength’ of the intramolecular structural constraint for a given probe without any additional synthesis whatsoever. This represents a significant improvement over the use of molecular beacons, where frequently many probes with different stem sequences must be synthesized and purified to find one with the proper duplex energy.

Affinity and specificity in the closed state are optional probe properties, in typical embodiments the probe in the closed state will possess affinity and specificity for the target molecule. The numerical values of the affinity and specificity (i.e. specificity factor) of the probe in the closed state will generally be in the same range as those described above for the probe in the open state.

Although the probe in the presence of metal ions will adopt the range of numerical values for affinity and specificity as outlined above, the affinity of the probe will generally be lower in the presence of metal ions than in their absence. While again not being bound by theory, this decrease in affinity is due to the fact that the binding of the target molecule requires the breaking of intra-probe associations that at least include the binding energy of the coordination complex, and therefore the overall binding energy of the target molecule to the probe will be reduced by an amount approximately equal to the energy of these internal associations. As such, the enhanced specificity exhibited by the constrained probes of the disclosure is accompanied by a minor reduction in their affinity relative to corresponding unconstrained probes having the same binding element. This minor reduction in affinity has not been found to be a shortcoming in practical use.

Numerous other structural modifications of the probe are provided according to the intended use of the probe (e.g. as an in vitro analytical reagent or as an in vivo pharmaceutical). In certain embodiments, one or more spacers (see “5.1 Glossary”) are provided between the binding element and one or both of the partial metal chelators, or between the partial metal chelators (or binding element) and the labels. A particular probe may thus have multiple spacers, which may, but need not, be identical to one another. Spacers are typically used to 1) separate the coordination complex in the closed state from the binding element to allow better access of the target molecule to the binding element, 2) separate the partial partial metal chelators and/or labels from the binding element in the probe-target complex to prevent any adverse effects they might have on the stabilty of the probe-target complex, 3) separate the partial metal chelators from the labels to prevent any undersired quenching of the label by the bound metal ions, 4) provide additional distance between the binding element and the partial metal chelators to permit them to form a coordination complex when the binding elements would otherwise be of insufficient length or flexibility to permit formation of the coordination complex, and 5) enhance the solubility of the probe. Other reasons for employing spacers in the probe will be apparent to those skilled in the art.

In other embodiments, the probes may be derivatized with one or more linkers (see “5.1 Glossary”) that are used to couple the probe to a solid-support. In such embodiments, the derivatized probe can be used to capture a target molecule on a solid-800 support such as a chromatography support, a microtiter dish, or on a microarray for the purpose of performing an analytical procedure. Microarray supports are particularly preferred since the probes offer enhanced specificity factors in microarray applications that are known in the art to suffer from poor specificity of target molecule capture.

In preferred embodiments, the resulting microarray comprises greater than 10, 100, 1000, 10000 or 100000 probes according to the disclosure. Each probe in the microarray preferably occupies an area between 1.0 cm² and 10⁻¹² cm². In some embodiments, the area occupied may be extremely small, being limited by the size of the individual probe itself. For example, a probe in the microarray may occupy an area less than about 10⁻¹ cm², 10⁻² cm², 10⁻³ cm², 10⁻⁴ cm², 10⁻⁵ cm², 10⁻⁶ cm², 10⁻⁸ cm², or 10⁻¹² cm².

In other embodiments, the detection of a target molecule either in solution or on a solid-phase is accomplished by modifying the probe and/or target molecule with one or a plurality of labels (see “5.1 Glossary”) to facilitate detection of target binding. For example, the probe may be derivatized with a label, such as, for example, a fluorophore or quantum dot. In array applications, typically the target is labeled. Such labeled probes may optionally also comprise one or more spacers between the labels and the probe. Labeled probes will find use as detection agents able to detect the presence of a molecule target in cellular extracts or within cells themselves, by administering the probe to the intracellular space.

In other embodiments the probe and/or target molecule are modified with interactive label pairs (see “5.1 Glossary”), which facilitate detection of the conformational change in the probe that is induced by target binding. For example, the probe may be modified with a FRET donor near the first partial metal chelator, and with a FRET acceptor near the second partial metal chelator. As the amount of fluorescence energy transfer between the donor and acceptor is inversely proportional to the distance between the donor and acceptor, the conformational change from the closed to the open state will result in a measurable reduction in acceptor fluorescence. In an alternative embodiment, the probe may be modified with a FRET donor near one partial metal chelator, and the target molecule modified with a FRET acceptor such that the donor and acceptor undergo energy transfer in the open state upon target molecule binding to the probe. In still another alternative embodiment, the probe may be modified with a FRET donor and acceptor near the first partial metal chelator, wherein properties of the binding element cause the donor and acceptor to associate with the binding element in the closed state and separate from one another providing some level of fluorescence or quenching (e.g. via stacking interactions with nucleobases in the binding element). Upon transformation to the open state, the donor/acceptor association with the binding element is abolished and the level of fluorescence or quenching detectably changes. In other embodiments, the FRET donor and acceptor may both be on the target molecule. The above labeling scenarios may also be carried out by substituting FRET donors and acceptors with other interactive label pairs such as dimerizing dyes (Packard et al. Proc. Natl. Acad. Sci. USA, 93:11640, 1996) and others described in further detail below. In certain embodiments, spacers are placed between the probe and interactive labels to facilitate interaction between the labels or inhibit undesirable interactions between the labels and the probe and/or the labels and the target molecule. Probes employing label pairs will find particular use as detection agents able to detect the presence of a molecule target in a homogeneous format.

In embodiments where the probe is used as an in vivo therapeutic (e.g. antisense, antigene, siRNA), the composition of the probe will be varied to optimize the absorption, distribution, metabolism and excretion of the probe (i.e. the “ADME” properties of the probe). In various embodiments, the probe is modified so as to increase or decrease the probe's hydrophobicity, number of H-bond donors and acceptors, and the number of rotatable bonds (see Veber et al., J. Med. Chem. 6:2615, 2002). In other embodiments the probe is modified to facilitate cellular uptake by, for example, covalently attaching folic acid to the probe so the probe is recognized by the folate receptor (see Low et al., 8^{th} International Symposium on Recent Advances in Drug Delivery Systems (1997), Salt Lake City, Utah, pp. 48-50 and Leamon et al., J. Biol. Chem. 268:24847, 1992). In other embodiments, the probe is modified with any of a variety of transduction domains to permit spontaneous entry into cells as reviewed by Dietz and Bahr, and incorporated herein by reference (Dietz and Bahr, Mol. Cell. Neurosci., 27:85, 2004). Optimizing the ADME properties employs testing probes with different compositions using methods well known to those skilled in the art. These methods include, but are not limited to, solubility testing; partitioning in biphasic mixtures of n-octanol and water; transport across Caco-2 and MDCK cell lines; binding to plasma proteins; degradation in plasma and whole blood; degradation in liver microsomes; degradation in isolated fractions of the P450 system; and monitoring and characterizing the absorption, distribution, metabolism and excretion of isotopically-labeled and unlabeled probes in rodent, canine, primate, and human species.

The probe can optionally comprise nucleophilic or electrophilic functional groups that can form covalent bonds with another pharmaceutical species such as a toxin or chemotherapeutic agent, so as to provide a means to target this pharmaceutical species to cells, tissues and tumors that produce a particular molecular target. Examples of suitable nucleophilic groups include amino, hydroxyl and sulfhydryl groups. Examples of suitable electrophilic groups include carboxyl, epoxide, ester, acid halide groups and their equivalents. Where the pharmaceutical species forms covalent bonds with the functional groups of the probe, the probe can comprise amino, hydroxyl or sulfhydryl groups which can formamide, ester or thioester bonds, respectively, with a carboxyl group, or its equivalent, of the pharmaceutical species, and vice versa. Similarly, epoxide-functionalized probes can form stable adducts with free amino, hydroxyl or sulfhydryl groups of the pharmaceutical species, and vice versa.

In one embodiment the pharmaceutical species is coupled to the probe. In a preferred embodiment, the pharmaceutical species coupled to the probe can be cleaved from the probe after administration to a patient, releasing the free pharmaceutical species. Covalent bonds suitable for in vivo cleavage of the pharmaceutical species will be well known to those skilled in the art. These include, but are not limited to, esters cleavable by non-specific intracellular esterases, GST-catalyzed cleavage of sulfones (see Gate and Tew, Expert Opin. Ther. Targets 5: 477, 2001), and GST-catalyzed cleavage of sulfonamides (see Zhao et al., Drug Metab. Dispos. 27:992, 1999).

In an alternative embodiment, the probes of the present disclosure may be labeled with one or more radioisotopes. The choice of radioisotope is based upon a number of well-known factors, for example, toxicity, biological half-life and detectability. Preferred radioisotopes include, but are not limited to ¹²⁵I, ¹²³I and ¹¹¹I. Techniques for labeling the probes of this disclosure are well known in the art. Most preferably, the radioisotope is ¹²³I and the labeling is achieved using ¹²³I-Bolton-Hunter Reagent. The labeled probe is administered to a patient and allowed to bind to one or more target molecules. The bound regions are then observed by utilizing well-known detecting means, such as a camera capable of detecting radioactivity coupled to a computer imaging system. Probes radiolabeled with isotopes suitable for positron emission tomography (PET) scanning are also preferred (e.g. ¹⁸F, ¹⁵O and ¹¹C). Radiolabeled probes will have use in diagnostic imaging of the distribution of target molecules in living patients.

The separate components of probes are described in further detail in the sections below:

### 5.3 The Binding Element

The probes comprise a binding element, through which they bind to an intended target molecule. The free binding element must have at a minimum, affinity for the target molecule (the properties of the whole probe have been described, see above). Preferably, the free binding element has an affinity for the target molecule that is characterized by a K_{d}, IC₅₀, Kₘ, or K_{I} whose numerical value is less than 1000 μM, 100 μM, 10 μM, and most preferably less than 1 μM. In embodiments where the binding element is a nucleobase polymer, the affinity for the target will be characterized by a Tₘ and will have a numerical value between 20° C. and 100° C., preferably between 30° C. and 90° C., more preferably between 40° C. and 80° C., and most preferably between 50° C. and 70° C. The affinity of the free binding element will not vary significantly as a function of the presence or absence of metal, and will typically be similar to the affinity of the whole probe in the absence of metal.

In addition to affinity for the target molecule, the free binding element must have specificity for the target molecule, characterized by a certain specificity factor (see “5.1 Glossary”). In embodiments where the specificity factor is characterized by a ratio of physical constants, the specificity factor of the free binding element is preferably greater than 1, than 2, than 10, and most preferably greater than 100. In embodiments where the free binding element is a nucleobase polymer, the specificity factor is greater than 2° C., preferably 5° C., more preferably 10° C., and most preferably greater than 20° C. The specificity factor of the free binding element will not vary significantly as a function of the presence or absence of metal, and will typically be similar to the specificity factor of the whole probe in the absence of metal.

The binding element is also of sufficient size and flexibility to allow it to adopt at least the open and closed conformations, where flexibility as used herein means that the binding element comprises a plurality of rotatable bonds. In preferred embodiments, the size of the binding element as measured in molecular weight is between 100-30,000 g/mol, 300-10,000 g/mol, and most preferably between 1,000-5,000 g/mol. Expressed as the number of monomers (i.e. amino acids or nucleobases) in a peptide or nucleobase polymer, the size of the binding element in preferred embodiments is between 2-100 monomers, 5-30 monomers, and most preferably 8-20 monomers.

The binding element can be of any chemical composition that meets these criteria of affinity, specificity and flexibility. By way of non-limiting examples, the binding element may comprise peptides, proteins, nucleobase polymers, nucleic acids, oligonucleotides, small-molecules less than 1,000 g/mol, multi-valent molecules, and polymers of natural amino acids, unnatural amino acids, peptoids, peptidomimetics, nucleobases, and carbohydrates, or combinations thereof. In preferred embodiments employing nucleobase polymers as the binding element, the affinity of the binding element for a particular target molecule can be reasonably predicted by simple inspection of the binding element for Watson-Crick (duplex) or Hoogsteen base pairs (triplex).

In embodiments where the affinity of the binding element cannot be reasonably predicted by simple inspection, potential molecules for use as binding elements can be screened using any methods known in the art, such as, for example, by using phage-display, ribosome-display, and in vitro aptamer evolution. In another representative method, potential binding elements can be screened by contacting a target molecule with a library of molecules. Methods for creating such molecule libraries are well known in the art. By way of example, library members can be screened either free in-solution or attached to solid-supports such as beads (e.g. PEG-PS, Novabiochem, Inc.) or attached to a flat-glass surface (i.e. a ‘chip array’ or ‘microarray’ or ‘array’).

The solubilized molecules from the library can then be screened for affinity to the target molecule using individual solution assays (e.g. using high-throughput chromogenic or FRET assays), by surface plasmon resonance employing the surface-bound molecule target (see Karlsson et al., Anal. Biochem. 278:1, 2000), or by contacting support-bound target molecule with the solubilized library members and then performing chromatography for subsequent isolation of bound members (see Shimizu et al., Nature Biotechnol. 18:877, 2000). Alternatively, the bead-bound library can be screened for affinity to the target molecule by contacting the support-bound library with fluorescently-labeled target molecule.

An additional requirement for a binding element is the ability to conjugate pendant groups to it in an appropriate fashion that the fully assembled probe will behave as described herein. The pendant groups that must be conjugated to it include at least two partial metal chelators, and optional labels, spacers, linkers, and other modifications as described above for the whole probe. Although affinity, specificity and flexibility are required properties of the binding element, the binding element itself need not provide a thermodynamic driving force to adopt the closed conformation; that is provided by the partial metal chelators discussed in further detail below.

### 5.4 Partial Metal Chelators

There are a wide variety of functional groups which can act as ligands to coordinate with a transition metal, the minimum requirement being the possession of an unshared electron pair (e.g. amines, thiols, carboxylic acids, and alcohols). Several guiding principles allow the further screening of useful ligand-metal binding pairs. Most prominently, preferred metal-ligand binding pairs may be identified phenomenologically. Therefore, ligands that bind preferentially to the metal in the presence of water will be initially preferred candidates in the present disclosure. Such ligands can be identified by noting the relative binding efficiencies of metals and metal complexes for such ligands or their analogs in the presence of water. An example of this principle is illustrated in the case of Ru(NH₃)₅.(H₂O) (ruthenium aquapentamine cation), which is well known to fix nitrogen in aqueous solution (Taube, et al., J. Am. Chem. Soc. 89:5706, 1967). Thus this complex is identified as a likely candidate for binding N-lewis base-containing ligands.

Another useful consideration for identifying candidates for screening effective metal-ligand binding pairs is afforded by the Hard-Soft-Acid-Base (HSAB) theory (J. Huheey, Inorganic Chemistry, Harper & Row (1972), p. 225-35). According to the HSAB theory “hard acids” have a preferred tendency to bind to “hard bases” while “soft acids” have a tendency to bind to “soft bases”. Thus metals in high oxidation states have a tendency based on their charge/radius ratios to bind to anions having high charge/radius ratios or to neutral ligands having relatively localized electron densities, e.g., hydrated metal ions and electropositive metals. On the other hand, soft acids, such as metals in their low or zero valance states have a tendency to bind to soft ligands that can accept electron density from the metal, e.g., metal carbonyl and metal-olefin compounds. The metal orbital constraints on binding presented by a given metal ion are a secondary consideration in determining efficient binding and generally will not present an obstacle to identifying efficient metal-ligand binding pairs. Instead, considerations of charge/radius ratio and polarizability of the ligand will predominate.

A chelator, by definition, is a moiety that possesses at least two ligands (e.g. bidentate, tridentate, tetradentate, etc.). By placing two or more such ligands in close proximity (and with appropriate geometry so that both may coordinate with a metal simultaneously), the binding affinity of the ligands for the metal is substantially greater than it would be for the ligands in isolation. This phenomenon is known as the ‘chelate effect.’ Without wishing to be limited by theory, the chelate effect is believed to arise from the additivity of favorable enthalpic binding terms without requiring multiple, unfavorable entropic terms. In practice, this means that, other factors being equal, the greater number of ligands a chelating group possesses, the higher overall binding affinity it will exhibit for a transition metal.

Although the maxium number of ligands that can be accepted by a transition metal varies with different transition metal ions, for many common transition metals the maximum number is six (e.g. nickel, cobalt, copper and zinc). With six ligands, a transition metal is said to possess an octahedral coordination geometry. The shape of 1030 such a complex resembles two four-sided pyramids, sharing the same base, with one pointing up and the other pointing down. Hence, the shape has eight sides (octahedral) with six corners. The present disclosure is not limited to metals possessing such geometry, and other metals and geometries are possible.

The chelators in the probes of the disclosure are strictly ‘partial metal chelators’ (see “5.1 Glossary”). Partial metal chelators within a probe must be chosen judiciously for their ability to ‘share’ a single transition metal ion. For example, EDTA would be an inappropriate choice for transition metals adopting an octahedral geometry, as it would fully occupy the metal and leave no available coordination sites to ‘share’ with a second chelator (i.e. EDTA is a complete metal chelator, see “5.1 Glossary”). In contrast, a probe could be constructed with a bidentate ligand at one partial metal chelator and a tetradentate ligand at the other partial metal chelator; the probe would thus fully occupy the octahedral geometry of the transition metal by the combination of the two partial metal chelators. A pair of tridentate ligands would also be appropriate. These examples are not meant to be limiting, however, and other arrangements, may also be employed to achieve a probe according to the present disclosure. These include those that provide less than the maximum number of ligands to a metal, such as a pair of bidentate ligands coordinated to a metal with an octahedral coordination geometry.

In identifying the pair of partial metal chelators most suited for use in a probe of the disclosure, one criterion will be that the partial metal chelators bind the selected metal atom with sufficient stability to promote the closed conformation in the absence of target, but permit the open conformation in the presence of target. Generally, the identification of preferred partial metal chelators for binding metal can be made by either first determining the partial metal chelators desired to be attached to the probe, then identifying metal ion centers that bind strongly to those partial metal chelators, or the desired metal ion may be first identified with preferred partial metal chelators identified subsequently. Thus, a preferred pair of partial metal chelators in a probe may be identified and then metal candidates screened for according to methods described in sections “5.6 Transition Metals” and “5.2 Probe Architecture and Properties”. Alternatively, partial metal chelators can be screened according to the methods in these sections following identification of a preferred metal ion.

The coordination complex is disrupted when the probe binds to target, but this does not necessarily mean the metal ion completely dissociates from the probe. The metal ion may remain chelated to one partial metal chelator or the other. Such a state is not incompatible with the probe-target complex, but nor is it required. It is also likely that a fraction of the disrupted probes also transiently contain two metal ions separately coordinated by each of the two partial metal chelators. In other words, the probe-target complex may contain metal ions coordinated to one or both of the partial metal chelators resulting in 1:1:1 or 2:1:1 metal:probe:target stoichiometries, but this is not essential to the functioning of the probe.

For an authoritative reference of partial metal chelators, see Perrin, D. D., Stability Constants of Metal-Ion Complexes Part B: Organic Ligands. Pergamon Press, Oxford: 1979, incorporated herein by reference in its entirety.

By way of non-limiting examples, suitable partial metal chelators may comprise those in TABLE 1 (where R in the table is the attachment point by which the partial metal chelator is covalently linked to the probe), and polymers presenting more than one imidazole, pyridine, pyrimidine, aniline, pyrrole, pyrazine, alkyl thiol or mercatobenzene moiety. Other suitable partial metal chelators will be apparent to one skilled in the art.

In other embodiments, suitable moieties for use in mediating conformational constraint of a binding element by complexation with a metal ion include macrocycles (calixarenes, crown ethers), carbene ligands and cyclophanes.

TABLE 1
Examples of Partial Metal Chelators

| Structure | molecule class | structure | molecule class |
|---|---|---|---|
| 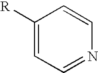 | pyridine | 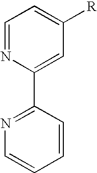 | bipyridine |
| 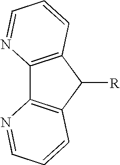 | methylenebipyridine | 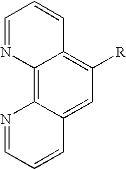 | phenanthroline |
| 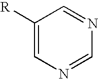 | pyrimidine | 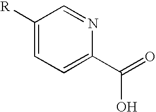 | functionalized(carboxy)pyridine |
| 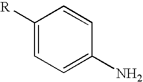 | analine | 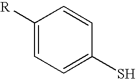 | phenylthiol |
| 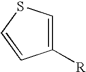 | thiophene | 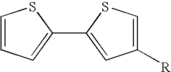 | bithiophene |
| 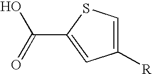 | functionalized(carboxy)thiophene | RSH | thiol |
| 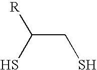 | dithiol | 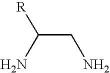 | diamine |
| 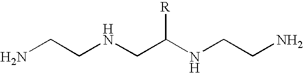 | poly (tetra)amine | 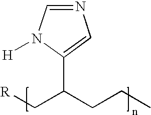 | polymer-imidazole |
| 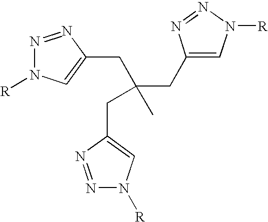 | branchedtriazole | 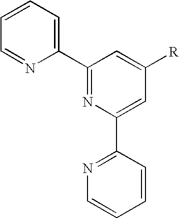 | terpyridine |
| 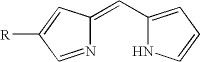 | hemiporphyrin-pyrrole | 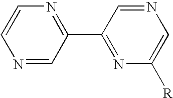 | bipyrazine |
| 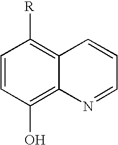 | hydroxyquinoline | 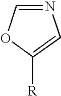 | oxazole |
| R₃P | phosphine | OP(OR)₃ | phosphate |
| 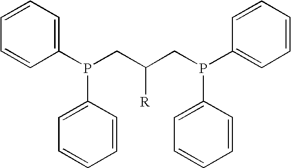 | bisphosphine | 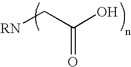 | aminoaceticacid |

In a preferred embodiment, both partial metal chelators of the probe are iminodiacetic acid (IDA) moieties, shown below.

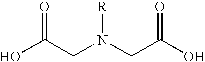

In this structure, ‘R’ is an attachment point by which the IDA group is covalently linked to the probe. The IDA moiety itself possesses three ligand sites to coordinate with a transition metal—two carboxylic acids and one tertiary amine. Thus, a probe that contains two IDA groups forms a coordination complex in which six ligands are contributed to the transition metal. Upon binding to target, this complex is disrupted and the two IDA groups become separated. The transition metal ion may remain coordinated with one of these IDA groups independently (with only three of its coordination sites occupied by the probe), or it may dissociate from the probe completely. Some transitory species where both IDA groups are coordinated with a metal ion are also possible.

In another embodiment, one of the partial metal chelators of the probe is nitrilotriacetic acid (NTA), shown below:

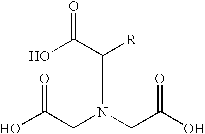

In this structure, ‘R’ is an attachment point by which the NTA group is covalently linked to the probe. NTA possesses four coordination sites-three carboxylic acids and one tertiary amine.

In still another embodiment, one of the partial metal chelators in the probe is a linear sequence of at least two histidyl residues, preferably linked together in a continuous polyhistidine track or in an interrupted polyhistidine track (i.e. a plurality of histidine residues separated from one another by other non-metal binding amino acid residues). One histidyl residue in the polyhistidine track is shown below where ‘R1’ and ‘R2’ represent either adjacent amino acids or an attachment point to the probe.

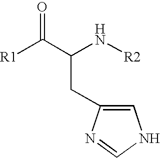

Although the histidyl side-chain is a heterocycle bearing two amine groups, it provides only a single ligand as the rigidity of the ring prevents both amines from coordinating to a single transition metal. In preferred embodiments, NTA or IDA as the first partial metal chelator is paired with a continuous or interrupted polyhistidine track as the second partial metal chelator on the probe, wherein the polyhistidine track contains from two to six histidyl residues.

The binding strength of the polyhistidine track to the metal ion can be modulated by varying its length and the number of histidyl residues it contains. Increasing the number of histidyl residues from two to six results in a proportionally stronger interaction with the metal ion. Increasing the length of the polyhistidine track also increases the interaction with the metal ion independent of the number of histidyl residues in the track. Without wishing to be bound by theory, it is believed that the flexibility of the peptide chain increases with lengths exceeding dipeptides, and this increased flexibility permits more favorable conformations for contributing histidyl ligands to the metal. For example, the increasingly longer and more flexible his-X-his, his-X—X-his and his-X—X—X-his motifs containing two histidyl residues exhibit greater metal binding affinity than the shorter but less flexible his-his dipeptide that also contains two histidyl residues (Arnold and Haymore, Science 252:1796, 1991).

The manner in which the partial metal chelators are covalently attached to the probe will depend upon the nature of the probe, as described more fully below (see section “5.7 Synthons and Methods of Probe Assembly”).

### 5.5 Labels

To assist in observing the binding of a probe to its target, probe-target complexes may be admixed with one or more labels that noncovalently bind the probe, target and/or the probe-target complex (e.g. SYBR green, ethidium bromide, or an antibody). In other embodiments, the probes will frequently contain covalently attached labels. Alternative embodiments may provide the target molecules with covalently attached labels, or provide both the probe and target molecule with covalently attached labels (e.g. as in an ‘interactive label pair’ of, for example, a FRET donor on the probe and a FRET acceptor on the target molecule, see “5.1 Glossary” and below). One skilled in the art will recognize a variety of such labels that could be employed, including radiolabels for detection by scintillation counters, fluorophores for optical detection, enzymes such as alkaline phosphatase or horseradish peroxidase, mass markers for detection by mass spectrometry (e.g. Patton, J. Chromatogr. B Analyt. Technol. Biomed. Life Sci., 771(1-2):3, 2002), and label moieties for the generation of light through radioluminescent, bioluminescent, chemiluminescent or electrochemiluminescent reactions.

In some embodiments, the labels are non-interactive, meaning that if one or a plurality of the same or different labels are included in a probe and/or target (covalently or noncovalently), they exhibit no change in a physically observable characteristic upon binding of the probe to its target. Non-interactive labels may be anywhere in the probe or target molecule, or may be conjugated to the probe or target molecule at any location, as long as the probe and target are able to bind one another, and the partial metal chelators are able to both simultaneously coordinate a single metal ion when the probe is not bound by target. Non-interactive labels are typically employed in heterogeneous detection embodiments, in which the labeled probe-target complex is separated from unbound probe and/or unbound target prior to detection.

In preferred embodiments, however, it is desirable for the probe to be used in a homogeneous detection format, in which the probe-target complex is detectable in the presence of unbound probe and/or unbound target. An interactive label pair (see “5.1 Glossary”) is a useful strategy to achieve a homogeneous detection format, wherein at least one of the labels of the pair undergoes a change in a physically observable characteristic (i.e. a ‘signal’) upon binding of the probe to target (an interactive label pair can also be used in a heterogeneous format). Preferably, the signal from an interactive label pair will be at least 1.1, 1.3, 1.5, 2.0, 5.0, 10.0, and most preferably at least 30.0 times greater when the probe is bound to its target than when the probe is unbound. In less preferred embodiments, an interactive label pair may generate a signal that is greater 1190 when the probe is in the unbound state than when the probe is bound to target.

In embodiments employing an interactive label pair, binding by target may for example, result in the interactive label pair exhibiting a change in excitation wavelength (λₑₓ), emission wavelength (λₑₘ), fluorescence quantum yield (Φ), fluorescence polarization (FP), and/or fluorescence lifetime (τ). These target-dependent changes may be the result of changes in, for example, the relative proximity of the labels (e.g. changes in τ), their chemical environment (e.g. changes in λₑₓ, λₑₘ, τ, and Φ) or their rotational rate in solution (e.g. changes in FP). According to other embodiments of an interactive label pair, a single fluorescent label is sufficient to constitute an interactive label pair. For example, λₑₓ, λₑₘ, τ, Φ and FP may all detectably change upon target binding by a probe bearing a single fluorophore label. In these embodiments, the probe and/or target can be considered to constitute the second ‘label’ in the ‘interactive label pair’, consistent with the definition of these terms as used herein (see “5.1 Glossary”).

Thus, one or a plurality of the same or different labels may be included in a probe and/or target (covalently or noncovalently), and constitute an interactive label pair, provided at least one label exhibits a change in a physically observable characteristic upon binding of the probe to its target. There is no requirement for a one-to-one correspondence between each type of label in an interactive pair, especially where one type can affect, or be affected by, more than one label of the other type (e.g. a probe may contain 5 of first label type, and 2 of a second label type, wherein one label of a first and second type together constitute an interactive label pair). Interactive label pairs may be anywhere in the probe and/or target molecule, or may be conjugated to the probe and/or target molecule at any location, as long as the probe and target are able to bind one another, and the partial metal chelators are able to both simultaneously coordinate a single metal ion when the probe is not bound by target.

Embodiments of noncovalent interactive label pairs include for example, SYBR Green and ethidium bromide, which bind a probe-target complex of duplex DNA with greater fluorescence (i.e. greater Φ) than free probe or target (Yguerabide and Ceballos, Anal Biochem. 228(2):208, 1995). In embodiments employing a single interactive label covalently attached to either the probe or target, probe- or target-linked thiazole orange, oxazole yellow or pyrene will bind a probe:target complex of DNA:DNA or DNA:PNA with a greater fluorescence (i.e. greater Φ) than either the free probe or target (Svanvik et al., Anal. Biochem. 281:26, 2000; Ishiguro et al., Nucleic Acids Res., 24: 4992, 1996; and Yguerabide et al., Anal. Biochem. 241(2):238-47, 1996). In other embodiments, a single interactive label will respond to a covalent modification of the probe by a target. For example, phosphorylation of an amino acid residue in the binding element by a target kinase results in its binding by a ‘chelating label’ and a resulting change in the label's spectral properties (Chen et al., J. Am. Chem. Soc., 124:3840, 2002).

In embodiments where the interactive label pair comprises a plurality of labels, binding of the target by the probe may result in changes in, for example, the relative proximity of the labels, their chemical environment, or the rotational rate of the probe-target complex in solution. For example, one label on the probe and another label on the target will result in the two labels coming into closer proximity upon formation of the probe-target complex (e.g., a FRET donor on the probe and a FRET acceptor on the target). In other embodiments, placing an interactive label next to each partial metal chelator in a probe will result in a significant increase in their spatial separation upon binding to target, since the partial metal chelators are held close together in the unbound probe but are physically separated upon binding of the probe to target. In these embodiments, preferred probe arrangements of an interactive label pair L₁ and L₂ with respect to the partial metal chelators PMC₁ and PMC₂ are L₁-PMC₁—PMC₂-L₂; PMC₁-L₁-PMC₂-L₂; L₁-PMC₁-L₂-PMC₂; and PMC₁-L₁-L₂-PMC₂; wherein at least a portion of the binding element lies between PMC₁ and PMC₂.

Other interactive label arrangements on the probe are possible when target-dependent changes are the result of other physical properties (e.g. changes chemical environment or rotational rate). For example, in some embodiments one or both of the labels in the pair bind the binding element in the absence of target, but in the bound probe-target complex remain free or bind to one another, resulting in a change in a physically observable characteristic (e.g. labels may bind into a nucleobase polymer binding element in the absence of target and be quenched by stacking interactions, but be unable to do so when the probe is bound to target). In these embodiments, preferred probe arrangements of an interactive label pair L₁ and L₂ with respect to the partial metal chelators PMC₁ and PMC₂ are L₁-L₂-PMC₁-PMC₁; and PMC₁—PMC₂-L₁-L₂; wherein at least a portion of the binding element lies between PMC₁ and PMC₂. In embodiments that employ binding-mediated changes in the electronic environment of an interactive label pair, it is apparent that the labels need not be maximally separated from one another on the probe, and in some embodiments may therefore be adjacent to one another.

The specific arrangement of the interactive labels required to observe the desired target-dependent changes may be determined empirically for a given interactive pair according to the screening methods described in sections “5.6 Transition Metals” and “5.2 Probe Architecture and Properties”, with the further screening constraint that target produces a detectable change in the interactive label pair. The use of spacers may assist in facilitating the desired arrangement as generally described in section “5.2 Probe Architecture and Properties”. These spacers may give the labels enough flexibility to interact with each other in the desired fashion.

An interactive label pair is in some embodiments a luminescent label and a quenching label, where the luminescent label is selected from a fluorescent label, a radioluminescent label, a chemiluminescent label, a bioluminescent label and an electrochemiluminescent label. The use of multiple quenching labels with a single luminescent label may increase quenching.

In preferred embodiments, an interactive label pair is a ‘fluorescence resonance energy transfer’ (i.e. FRET) pair. The efficiency of energy transfer in a FRET pair from the donor to the acceptor is dependent upon the spatial separation between them, and hence varies between the ‘open’ and ‘closed’ conformations. In some embodiments, the energy accepting label of the pair may be a dark quencher (e.g. DABCYL) with no fluorescent properties, in which case the observable signal is simply the fluorescence output of the energy donor label. For example, a probe labeled with EDANS adjacent to one partial metal chelator and DABCYL adjacent to the other partial metal chelator will efficiently transfer energy between these labels when the probe is in the closed state (i.e. DABCYL quenches the EDANS fluorescence). When EDANS is excited by an appropriate frequency of light, a fluorescent signal is generated from the probe at a first level, which in some embodiments may be zero (i.e. ‘off’). When the probe is in the open state after binding target, the EDANS label is sufficiently separated from the DABCYL label that fluorescence resonance energy transfer between them is substantially, if not completely, abolished. If EDANS is excited, a fluorescent signal of a second level, higher than the first is generated (i.e. ‘on’). The difference between the two levels of fluorescence is detectable and measurable, and its presence indicates that the probe is bound to the target.

In other embodiments, the FRET pair is two spectrally-overlapping fluorophores, where the fluorescence of both the donor and acceptor will be different depending upon the probe conformation. For example, using fluorescein—rhodamine as the donor—acceptor pair, excitation of fluorescein will result in observable fluorescence at the emission wavelengths of both fluorophores. When the probe is in the closed state, the efficiency of energy transfer is high, and therefore the fluorescence from rhodamine will be relatively high, and that from fluorescein will be relatively low. When the probe shifts to the open state, the efficiency of energy transfer is reduced, resulting in an increase in the fluorescence emission of fluorescein and a corresponding decrease in the fluorescence emission of rhodamine. In this fashion, two fluorescent signals can be used to indicate the conformational state of the probe, and its binding to target. The ratio between those two signals may also be calculated.

In alternative quenching embodiments, the interactive label pair is a pair of identical fluorphore labels that undergo self-quenching. For example, TMR label pairs are one of many fluorophore pairs known in the art to exhibit ‘self-quenching’. By placing TMR label pairs adjacent to the two chelation sites so that they will be in contact with one another and quenched when the probe is in the closed state, the fluorescence intensity of the TMR fluorophore increases upon target binding due to the separation of the two TMR moieties.

In still other alternative quenching embodiments, interactive label pairs undergo collisional quenching and therefore do not require spectral overlap between each label as required in other FRET pairs. By placing each label in the pair close to each partial metal chelator, the labels physically contact one another (i.e. “collide”) in the closed state, resulting in highly efficient fluorescence quenching. For example, DABCYL effectively quenches a variety of fluorophores by collisional quenching, despite having no spectral overlap. DABCYL is therefore a preferred quencher, and interactive label pairs that undergo collisional quenching can be prepared by pairing DABCYL with many common fluorophores. In a preferred embodiment, quenching in the closed state is enhanced by a single fluorophore near one partial metal chelator and multiple DABCYLs near the other partial metal chelator, such that at least one DABCYL is adjacent to the fluorophore in the coordination complex.

In another proximity-modulated embodiment, an interactive label pair may comprise two pyrene moieties able to interact with one another and form a pyrene-pyrene excited state dimer (i.e. an ‘excimer’) in the closed state, but not in the open state. The pyrene excimer in the closed state yields a fluorescent emission that is detectably different than the separate pyrene monomers in the open state (longer Rem and greater intensity). The ratio between those two signals may also be calculated.

In use, labels can be excited with a narrow wavelength band of radiation or a wide wavelength band of radiation. Similarly, the emitted radiation can be monitored in a narrow or a wide range of wavelengths, either with the aid of an instrument or by direct visual observation. The choice of fluorophore is dependent upon the requirements of the method of use. For example, if a probe is to be used in a fluorescence detection instrument that uses an argon laser as its excitation source, then a fluorophore which can be efficiently excited by the wavelenths of light from this laser must be used. In this case, fluorescein and its derivatives would be appropriate choices. If multiple probes (for binding to different targets) are to be used in a single homogeneous detection format, then they must be spectrally distinguishable by the instrument with which the method will be performed.

### 5.6 Transition Metals

According to certain embodiments, a probe requires a transition metal ion (i.e. a ‘metal ion’) to function, wherein the metal ion is coordinated to one or both of the partial metal chelators of the probe. The metal ion is a separate molecular entity from the probe (i.e. binding element and partial metal chelators, and optional labels, linkers and spacers). A metal ion suitable for use with a probe of this disclosure can be any metal ion capable of coordinating simultaneously to both partial metal chelators of the probe, so long as the simultaneous coordination is disrupted upon target binding.

Typical metal cations include, for example, zinc (Zn), cadmium (Cd), copper (Cu), nickel (Ni), ruthenium (Ru), platinum (Pt), palladium (Pd), cobalt (Co), magnesium (Mg), barium (Ba), strontium (Sr), iron (Fe), vanadium (V), chromium (Cr), manganese (Mn), rhodium (Rh), silver (Ag), mercury (Hg), molybdenum (Mo), tungsten (W), calcium (Ca), lead (Pb), cerium (Ce), aluminum (Al) and thorium (Th). The ionic state of metal ions can vary, as is well known, and it is preferred that the oxidation state of a metal cation in a probe of this disclosure be one where the oxidation state is indicated in parenthesis: Zn(II), Cd(II), Cu(I), Cu(II), Ni(II), Ru(II), Ru(III), Pt(II), Pd(II), Co(II), Co(III), Mg(II), Ba(II), Sr(II), Fe(II), Fe(III), V(III), Cr(II), Cr(III), Mn(II), Rh(III), Ag(I), Hg(II), Mo(III), Mo(IV), Mo(V), Mo(VI), W(III), W(IV), W(V), W(VI), Ca(II), Pb(II), Ce(III), Al(III), and Th(IV). The metal ion may also be radioactive or paramagnetic, and in some embodiments a medically useful metal ion.

The identity of the metal ion is an important consideration for the proper function of the probe, but no single metal cation is necessarily required for a particular probe to properly function. For a given probe, the strength holding the probe in the closed conformation provided by the binding energy of the coordination complex varies among different transition metals. In general, the strength holding the probe in the closed conformation is sufficient to provide a stable coordination complex in the absence of target. Stability of the coordination complex is indicated by the conditions under which the ligands of a coordination complex disassociate from the metal ion in the coordination complex, and reassociate with either a different metal ion or the original metal ion. The process of dissociation and reassociation of a metal with a coordination complex is referred to as ‘exchange’. In relative terms, stable complexes are more exchange-inert, and less stable complexes are less exchange-inert (i.e. more exchange-labile). For a general discussion of exchange in metal complexes, see Taube, Chem. Rev., 50:69, 1952; Van Wart, Meth. Enzymol., 158:95, 1988; Barton, Comm. Inorg. Chem., 3:321, 1985; Metal-Ligand Interactions in Organic Chemistry and Biochemistry, Pullman, et al., Eds., D. Reidel, Boston (1977); Margalit, et al., J. Am. Chem. Soc., 105:301, 1983; and Friedman, et al., J. Amer. Chem. Soc., 112:4960, 1990.

A metal ion that forms a relatively exchange-inert coordination complex, i.e., a relatively stable coordination complex, is preferred over metal ions that form exchange-labile or relatively unstable complexes, because the resulting probe is more likely to maintain the closed conformation in the absence of target. In general, a metal ion that is more exchange-inert in a coordination complex will impart a lower target-affinity on the probe than a metal ion that is less exchange-inert. Thus, the relative exchange of the probe coordination complex as a function of the type of metal ion can be determined by separately admixing the probe with different metal ions, and then separately measuring the target-affinity of each of the admixtures. According to this method, the relative strength and stability of the coordination complex as a function of the type of metal ion employed will follow the same relative trend as the degree to which the coordination complex is exchange-inert.

For example, it was discovered that the target affinity of a probe with two IDA partial metal chelators varied as a function of the type of metal ion according to the trend: Ni(II)<Cu(II)=Co(II)<Zn(II)<Fe(II)=Mn(II). Thus, the degree to which the coordination complex is exchange-inert as a function of the metal ion follows the trend: Ni(II)>Cu(II)=Co(II)>Zn(II)>Fe(II)=Mn(II). Similarly, the relative stability or strength of the probe coordination complex as a function of the type of metal ion are said to follow the trend: Ni(II)>Cu(II)=Co(II)>Zn(II)>Fe(II)=Mn(II). In a further 1410 example highlighting the trend variability as a function of the types of partial metal chelators employed, the stability of the coordination complex in a probe with an NTA partial metal chelator and a polyhistidine partial metal chelator has been found to follow the opposite trend: Cu(II)>Ni(II).

In addition to stability of the coordination complex per se, there must also be a proper balance between the binding energy of the coordination complex and the binding energy of the probe-target complex. In so far as the ligands provided by the partial metal chelators form the metal binding site in the closed conformation, the ligands, their geometry in the closed probe conformation, and their chemical properties all contribute to establishing the balance between these energies for a particular metal ion. Thus, the most preferred metal ions provide a coordination complex which is both maximally exchange-inert, and possessing a binding energy equal to, or less than the binding energy of the target-probe complex. Less preferred metal ions provide a coordination complex that is relatively exchange-labile with a binding energy that exceeds the binding energy of the target-probe complex.

Different transition metals may be readily screened to identify those that are most suitable for use with a given probe by separately mixing an aliquot of the probe with one of a variety of different transition metals. Those metal ions that are most suitable for use with the probe will exhibit the physical properties specified above in section “5.2 Probe Architecture and Properties”.

While the probe is preferably assembled by solution-phase or solid-phase synthesis (see below), the closed conformation is generated by simply admixing a salt of the metal ion (commercially available) with the probe in aqueous solution. An important consideration in preparing this admixture is the concentration of the metal ion to be used with the probe. Not to be bound by theory, it is thought that the formation of the closed probe conformation is a two step process, wherein the metal first binds to one of the partial metal chelators followed by a rapid and thermodynamically favorable intramolecular binding of the other partial metal chelator. Within this paradigm where the first step is limiting, it is predicted that the minimum suitable probe and metal concentrations will be those that efficiently generate the first probe-metal binding event. That is, the concentrations of the probe and metal ion should at least approximate the metal dissociation constant of the partial metal chelator (K_{pmc}). However, this paradigm also predicts that concentrations of probe and metal that greatly exceed K_{pmc} and employ a large excess of the metal ion will likely form inactive probes, wherein the closed conformation is precluded by a probe-bound metal:probe stoichiometry of 2:1. Thus, in preferred embodiments it is desirable that the probe and metal ion concentrations should at least approximate K_{pmc}, and the stoichiometry of metal ions to probe ranges from about 10:1 to 1:10. Most preferably however, the stoichiometry of metal ions to probe will be as close as possible to 1:1. In other embodiments though, if the probe and/or metal concentrations are very low relative to K_{pmc}, then excess metal can be used to ensure that the stoichiometry of probe-bound metal ions to probe are in the range of from about 10:1 to 1:10, and most preferably as close to 1:1 as possible.

In practice, probes having two IDA partial metal chelators will completely form the closed coordination complex when incubated with nickel or copper ions equimolar with the probe and in a concentration range of 100 nM to 500 nM. Addition of a small excess of metal ions (up to about 4-fold) does not appreciably affect the affinity of probe-target binding. However, addition of a large excess (greater than 8-fold) results in changes to the affinity of probe-target binding suggestive of the inactive 2:1 metal:probe complexes. Probes having one NTA partial metal chelator and one polyhistidine partial metal chelator also completely form the closed coordination complex when incubated with nickel or copper ions equimolar with the probe and in a concentration range of 100 nM to 500 nM. However, the excess nickel required to form the inactive 2:1 metal:probe complex depends on the length of the polyhistidine partial metal chelator. With a hexahistidine partial metal chelator, inactive 2:1 complexes begin to form with as little as a 4-fold excess of nickel. However, probes with a dihistidine partial metal chelator show no evidence of the inactive 2:1 complex even with an 8-fold nickel excess, presumably reflective of the larger K_{pmc} of the dihistidine versus the hexahistidine partial metal chelator.

When using probes at concentrations below 100 nM, it is possible that equimolar metal ions will not be sufficient to attain complete coordination of the probe. It is probable, however, that a small excess (2- to 4-fold) of metal ions will ensure complete formation of the closed coordination complex. One skilled in the art may readily test a range of transition metal concentrations to identify a suitable concentration range of metal ions for use with a given probe concentration by varying the concentration of the metal ion and monitoring the binding affinity of the probe to target as a function of metal ion concentration by measuring, for example, the probe-target Tₘ, K_{d}, IC₅₀, Kₘ, and K_{I} (see Example 6.3.2). Evidence of the inactive 2:1 complex is obtained when the probe-target binding affinity in the presence of the metal ion is substantially equivalent to the affinity in the absence of the metal ion. Those probe and metal ion concentrations that are most preferable exhibit a probe-target binding affinity less than, and most preferably maximally less than, the probe-target binding affinity in the absence of the metal, while at the same time having the physical properties specified in section “5.2 Probe Architecture and Properties”.

Transition metal ions are typically provided as salts with a variety of counterions. Two common counterions are chloride and sulfate. No difference has been observed in the behavior of probes prepared with these two different counterions for a given transition metal. Salts of other counterions are also available, and it is expected that the identity of the counterions will in general not affect probe function.

### 5.7 Synthons and Methods of Probe Assembly

In general, probes can be synthesized by the condensation of synthons. Synthons are chemical building blocks that contribute to the binding element and partial metal chelators of the probes, as well as to the optional probe labels, spacers and linkers. Synthons bear one or more reactive moieties that include, but are not limited to azide, carboxyl, acid chloride, hydroxyl, amino, thiol, thioester, alkylene, alkyne, aryl halide, alkyl halide, phosphoramidite and H-phosphonate. Synthons are typically condensed in-solution or on the solid-phase after modification by protection with suitable protecting groups, by activation with activators, or by a combination thereof. In some embodiments, synthons are condensed in-solution or on the solid-phase without modification.

### Protecting Groups

Suitable protecting groups are those known to be useful in the art of stepwise solid-phase synthesis and solution-phase synthetic chemistry. Included are acyl type protecting groups (e.g., formyl, trifluoroacetyl, acetyl, phenoxyacetyl, p-isopropyl-phenoxyacetyl), aryl type protecting groups (e.g., biotinyl), aromatic urethane type protecting groups (e.g., benzyloxycarbonyl (Cbz), substituted benzyloxycarbonyl and 9-fluorenylmethyloxy-carbonyl (Fmoc)), aliphatic urethane protecting groups (e.g., t-butyloxycarbonyl (BOC), isopropyloxycarbonyl, cyclohexyloxycarbonyl) and alkyl type protecting groups (e.g., benzyl, triphenylmethyl and methoxy derivatives thereof). Other protecting groups will be known to those skilled in the art and include, for example, those disclosed in T. W. Greene and P. G. M. Wuts, Protective Groups in Organic Synthesis, 3^{rd} Edition, John Wiley & Sons, New York, 1999. Two protecting groups that are removed by conditions that are different from one another are said to be ‘orthogonal’ protecting groups, and are ‘orthogonal’ to one another.

### Activators

Suitable activators for synthons in acyl couplings include, but are not limited to, DCC, DIPCDI, 2-chloro-1,3-dimethylimidium hexafluorophosphate (CIP), benzotriazol-1-yl-oxy-tris-(dimethylamino)-phosphonium hexafluorophosphate (BOP) and its pyrrolidine analog (PyBOP), bromo-tris-pyrrolidino-phosphonium hexafluorophosphate (PyBroP), N-[(1H-benzotriazol-1-yl)-(dimethylamino) methylene]-N-methylmethanaminium hexafluorophosphate N-oxide (HBTU) and its tetrafluoroborate analog (TBTU) or its pyrrolidine analog (HBPyU), (HATU) and its tetrafluoroborate analog (TATU) or pyrrolidine analog (HAPyU). Common catalytic additives used in acyl coupling reactions include 4-dimethylaminopyridine (DMAP), 3-hydroxy-3,4-dihydro-4-oxo-1,2,3-benzotriazine (HODhbt), N-hydroxybenzotriazole (HOBt) and 1-hydroxy-7-azabenzotriazole (HOAt). In some embodiments directed to difficult acyl couplings, activators will be used that yield an acid fluoride or chloride derivative of a synthon. Suitable activators for synthons employed in preparing internucleotidyl linkages will be familiar to those in the art, and will depend on whether the synthon condensation reactions employ, for example, phosphotriester/phosphoramidite (e.g. tetrazole; 4,5-dicyanoimidazole; 5-ethylthio-1H-tetrazole; and 5-benzylthio-1H-tetrazole activators) or H-phosphonate (e.g. pivaloyl chloride and 1-adamantanecarbonyl chloride as activators) coupling chemistries. Other activators will be known to those skilled in the art.

### Binding Element Synthons

Synthons suitable for assembly of the binding element may be readily determined by one skilled in the art of synthetic chemistry. For example, in some embodiments synthons suitable for assembly of a peptide binding element include, but are not limited to, natural and unnatural amino acids and the D or L optical isomers as described in Stewart et al., Solid Phase Peptide Synthesis, 2^{nd} Edition, Pierce Chemical Co., Rockford, Ill., 1984; Bayer & Rapp, Chem. Pept. Prot. 3:3, 1986; Atherton, et al. Solid Phase Peptide Synthesis: A Practical Approach, IRL Press, Oxford, 1989; Barany and Merrifield, Solid-Phase Peptide Synthesis, pp. 3-284 in The Peptides: Analysis, Synthesis, Biology. Vol. 2: Special Methods in Peptide Synthesis, Part A; Merrifield, et al. J. Am. Chem. Soc. 85:2149, 1963; and the Novabiochem 2003/04 Catalog, Novabiochem, San Diego, Calif.).

In other embodiments, binding element synthons are ‘peptidomimetics’, wherein such synthons may have steric, electronic or configurational properties similar to a natural amino acid, but such similarities are not necessarily required. Peptidomimetic synthons are often used in the assembly of the binding element to inhibit degradation of the probe by enzymatic or other degradative processes. Peptidomimetic synthons can be produced by organic synthetic techniques. Examples of suitable peptidomimetic synthons include tetrazol (Zabrocki et al., J. Am. Chem. Soc. 110:5875, 1988); isosteres of amide bonds (Jones et al., Tetrahedron Lett. 29:3853, 1988); LL-3-amino-2-propenidone-6-carboxylic acid (LL-Acp) (Kemp et al., J. Org. Chem. 50:5834, 1985). Similar analogs are shown in Kemp et al., Tetrahedron Lett. 29:5081, 1988, as well as in Kemp et al., Tetrahedron Lett. 29:5057, 1988, Kemp et al., Tetrahedron Lett. 29:4935, 1988, and Kemp et al., J. Org. Chem. 54:109, 1987. Other suitable peptidomimetics are shown in Nagai and Sato, Tetrahedron Lett. 26:647, 1985; Di Maio et al., J. Chem. Soc. Perkin Trans., 1687 (1985); Kahn et al., Tetrahedron Lett. 30:2317, 1989; Olson et al., J. Am. Chem. Soc. 112:323, 1990; Garvey et al., J. Org. Chem. 56:436, 1990. Further suitable peptidomimetics include hydroxy-1,2,3,4-tetrahydroisoquinoline-3-carboxylate (Miyake et al., J. Takeda Res. Labs 43:53, 1989); 1,2,3,4-tetrahydroisoquinoline-3-carboxylate (Kazmierski et al., J. Am. Chem. Soc. 133:2275, 1991); histidine isoquinolone carboxylic acid (HIC) (Zechel et al., Int. J. Pep. Protein Res. 38(2):131-138, 1991); (2S,3S)-methyl-phenylalanine, (2S,3R)-methyl-phenylalanine, (2R,3S)-methyl-phenylalanine and (2R, 3R)-methyl-phenylalanine (Kazmierski and Hruby, Tetrahedron Lett., 32(41):5769, 1991). In other embodiments, the peptidomimetics are peptoid synthons (Simon et al., Proc. Natl. Acad. Sci. USA, 89:9367, 1992).

In other embodiments, synthons suitable for assembly of a nucleobase polymer binding element include, but are not limited to, natural and unnatural DNA and RNA synthons (see Dorman, Noble, McBride, & Caruthers Tetrahedron 40:95, 1984; Adams, Kavka, Wykes, Holder, & Galluppi, J. Am. Chem. Soc. 105:661, 1983; Matteucci, & Caruthers, J. Am. Chem. Soc. 103:3185, 1981, Beaucage & Caruthers Tetrahedron Lett. 22:1859, 1981, and the 2006 Glen Research Catalog, Glen Research, Sterling, Va.), PNA synthons (see Buchardt et al., PCT WO 92/20702; Coull et al., PCT WO 96/40685 and Buchardt et al., U.S. Pat. No. 5,719,262), morpholino-based synthons (see Summerton and Weller, U.S. Pat. No. 5,698,685; Summerton et al., U.S. Pat. No. 5,378,841 and Summerton and Weller, U.S. Pat. No. 5,185,444), PENAM synthons (see Shah et al., U.S. Pat. No. 5,698,685), and synthons providing polynucleosides with linkages comprising carbamate (see Stirchak and Summerton, J. Org. Chem. 52:4202, 1987), amide (see Lebreton et al., Synlett. February 1994:137), methylhydroxylamine (see Vasseur et al., J. Am. Chem. Soc. 114:4006, 1992), 3′-thioformacetal (see Jones et al., J. Org. Chem. 58:2983, 1993), sulfamate (see Huie and Trainor, U.S. Pat. No. 5,470,967) and others (see Swaminathan et al., U.S. Pat. No. 5,817,781 and Freier and Altmann, Nucl. Acids Res. 25:4429, 1997, and references cited therein).

### Partial Metal Chelator Synthons

As used herein, the term ‘partial metal chelator synthon’ means a synthon that contains at least one partial metal chelator, wherein the partial metal chelator synthon may be unprotected or protected by one or more protecting groups. In certain embodiments, a partial metal chelator synthon comprises a first and second protecting group, wherein the partial metal chelator is protected by the first protecting group, and the first and second protecting groups are orthogonal to one another. Such a partial metal chelator synthon may further comprise a transition metal ion, a label, or a third protecting group which is orthogonal to both the first and second protecting groups. Protecting groups protect one or more reactive moieties on the partial metal chelator synthons that include, but are not limited to carboxyl, hydroxyl, amino, thiol and phosphoramidite.

Synthons suitable for providing the partial metal chelator may be readily determined by one skilled in the art of synthetic chemistry. Preferably, synthons will permit their assembly either in-solution or, more preferably, on the solid-phase. In other preferred embodiments, partial metal chelator synthons will be coupled using the same repetitive coupling chemistry as the remaining synthons used to prepare the complete probe. For example, probes with peptide or PNA binding elements will preferably be prepared using partial metal chelator synthons that are amino acids, and probes with DNA or RNA binding elements will preferably be prepared using partial metal chelator synthons that are phosphoramidites or H-phosphonates.

Thus, in some embodiments the partial metal chelator synthons are amino acids. Typically, the partial metal chelator (e.g. IDA, NTA) will be attached through a plurality of chemical bonds to the alpha carbon of the amino acid, and the first protecting group will comprise an Fmoc or BOC protecting group attached to the alpha amino group. In some embodiments the amino acid synthon is an oligopeptide of di-, tri-, tetra-, penta- or hexa-histidine (note that histidine is not a partial metal chelator synthon as used herein, since it does not provide a plurality of coordination ligands).

In other embodiments the partial metal chelator synthons are amino acid derivatives of the partial metal chelators disclosed in TABLE 1 (see section ‘5.4 Partial Metal Chelators’). Methods for preparing partial metal chelator synthons of Fmoc- or BOC-protected natural amino acids, unnatural amino acids and peptidomimetics will be apparent to those skilled in the art, using for example, methods as generally described in Kazmierski, Int. J. Peptide Protein Res., 45:241, 1995; Kazmiershi, Tet. Lett., 34:4493, 1993; Ruan et al., J. Org. Chem., 56:4347, 1991; Ruan et al., J. Am. Chem. Soc., 112:9403, 1990; and U.S. Pat. Nos. 5,200,504 and 6,531,572.

Preferred embodiments of partial metal chelator synthons that are amino acids include the following:

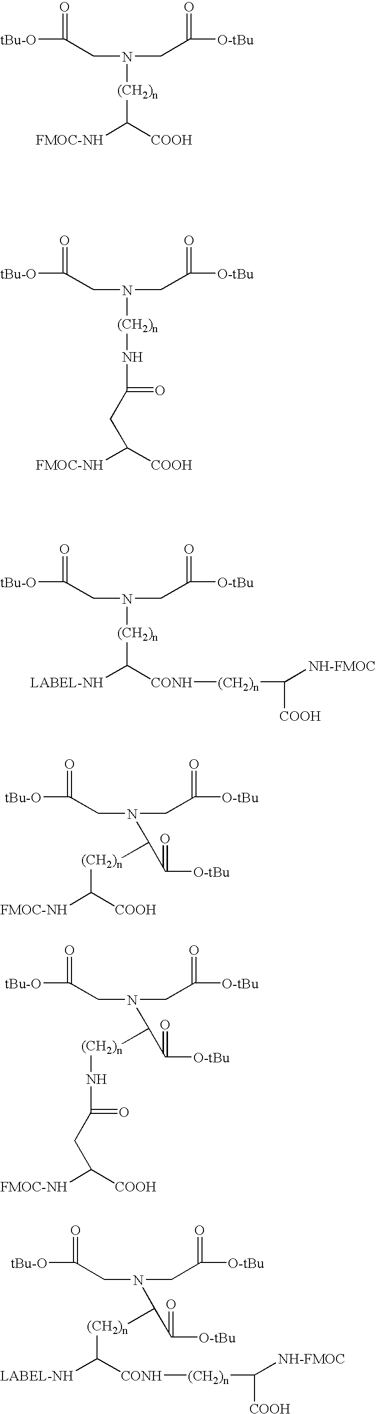

where n is independently 0 or an integer 1-5, ‘tBu’ is tert-butyl, and ‘LABEL’ is a label as provided in section ‘5.5 Labels’. The construction of such partial metal chelator synthons can be readily accomplished by the derivatization of commercially available protected amino acid precursors with, for example bromo-tert-butylacetate and optionally one or more other amino acids or labels (e.g. ‘LABEL’ in the above structures). Reacting bromo-tert-butylacetate with suitably protected amino acids bearing a free amine provides the IDA moiety, while reacting bromo-tert-butylacetate with a free α-amine on any of a variety of α-amino acids provides the NTA moiety (e.g. see methods in the Examples).

In still other embodiments the partial metal chelator synthons are phosphoramidites or H-phosphonates. In these embodiments, the partial metal chelator synthons are for example, phosphoramidite or H-phosphonate derivatives of the partial metal chelators disclosed in TABLE 1 (see section ‘5.4 Partial Metal Chelators’). The following methods are known for preparing phosphoramidites of partial metal chelators that consist of: terpyridine (Zapata et al., Eur. J. Org. Chem., page 1022, 2003; Bianke and Haner, ChemBioChem, 5:1063, 2004); thiopenol (Hatano et al., Tetrahedron, 61:1723, 2005); 2-aminopehenol, catechol, o-phenylenediamine (Shionoya and Tanaka, Bull. Chem. Soc. Jpn., 73:1945, 2000); pyridine-2,6-dicarboxylate, pyridine (Meggers et al. J. Am. Chem. Soc., 122:10714, 2000); 2,2′-bipyridine, phenanthrene and 1670 phenanthroline (Weizman and Tor, J. Am. Chem. Soc., 123:3375, 2001; Bianke et al., Bioconjugate Chem., 17:1441, 2006; Langenegger and Haner, Tetrahedron Letters, 45:9273, 2004); 2-pyrido-2-benzimidazole, 2-quino-2-benzimidazole (Kim and Kool, J. Am. Chem. Soc., 128:6164, 2006); pyridylpurine (Switzer et al., Angew. Chem. Int. Ed., 44:1529, 2005); pyridine-2,6-dicarboxamide (Zimmermann et al., Bioorganic Chemistry, 32:13, 2004); 4-(2′-pyridyl)-pyrimidinone (Swizer and Shin, Chem. Commun., page 1342, 2005); 8-hydroxyquinoline (Zhang and Meggers, J. Am. Chem. Soc., 127:74, 2005); hydroxypyridone (Tanaka et al., J. Am. Chem. Soc., 124:12494, 2002); 2,6-bis(ethylthiomethyl)pyridine (Zimmerman et al., J. Am. Chem. Soc., 124: 13684); bis-imidazole (Vargas-Baca et al., Angew. Chem. Int. Ed., 40(24):4629, 2001); 2, 6-1680 pyridinedicarboxylate and 2,2′-dipicolylamine (Kady and Groves, Biorg. Med. Lett., 3(6): 1367, 1993). Also known are methods for preparing phosphoramidite precursors to the on-probe in situ generation of a partial metal chelate (Clever et al., Chem. Eur. J., 12:8708, 2006).

In alternative embodiments the partial metal chelator will attach to a nucleobase or sugar in the phosphoramidite or H-phosphonate synthon, and these may be prepared using methods such as those described in U.S. Pat. Nos. 4,795,700 and 4,837,312. For example, aliphatic substitution of a partial metal chelator at the fifth carbon of pyrimidine nucleobases can be achieved by palladium mediated olefination reactions as described by Bergstrom and Ruth, J. Carbohydrates Nucleosides Nucleotides 4:257, 1977; Bergstrom, & Ogawa, J. Am. Chem. Soc. 100:8106, 1978; Heck, J. Am. Chem. Soc. 90:5518, 1968; and Langer, Waldrop, & Ward, Proc. Natl. Acad. Sci. USA, 78(11):6633, 1981. Palladium (II) coupling chemistry has been used to introduce alkyl side chains at carbon five of uridine, deoxyuridine, cytosine and deoxycytosine via the carbon five mercury or halogen derivatives as described by Langer et al., Proc. Natl. Acad. Sci. USA, 78(11):6633, 1981.

The partial metal chelator may also be attached at carbon eight of purine nucleobases (e.g. adenosine, guanosine, deoxyadenosine, deoxyguanosine nucleosides) and at carbon seven of deaza nucleobases (e.g. deazaadenosine and deazaguanosine nucleosides). Purine nucleobases, such as adenine and guanine are halogenated, and mercurated at carbon eight. Mercuration and halogenation also occur at carbon seven of 7-deazapurine nucleoside derivatives (Holmes and Robins, J. Am. Chem. Soc. 86:1242, 1963). Alkyl side chains are introduced in these halogen and mercury derivatives by palladium II coupling chemistry (Dale et al., Proc. Natl. Acad. Sci. USA, 70:2238, 1973). Direct free-radical alkylation is also used to introduce alkyl side chains containing a partial metal chelator at carbon eight of the purine nucleosides (Christenson et al., Biochem. 14(7):1490, 1975).

The partial metal chelator is also attached to amino derivatives of nucleosides at the 5′ carbon atom of the ribose (Delaney, et. al., J. Carbohydrates, Nucleosides, Nucleotides, 8 (5):445, 1981). Alternatively, the partial metal chelator may be attached to hydrocarbon chains at nitrogen four of cytidine and deoxycytidine nucleosides, using bisulfite-catalyzed transamination to introduce a 3-aminopropylside chain at carbon four (Draper and Gold, Biochem. 19:1774, 1980). In addition, the partial metal chelator may be attached at nitrogen four using 4-thiouridine (Smrt, Neoplasma, 24:461, 1977). A partial metal chelator attached to a hydrocarbon tether is also incorporated at nitrogen six of adenosine and deoxyadenosine, and at nitrogen two of guanosine and deoxyguanosine using reduction amination (Borch, et. al., J. Am. Chem. Soc. 93:2897, 1971).

The tethered partial metal chelatoris attached to an internucleotidyl phosphate of polynucleotides (Asseline et al., C.R. Acad. Sc. Paris, 297:369, 1983; and Proc. Natl. Acad. Sci. USA, 81:3297, 1984). The partial metal chelator is also tethered to uridine 3′ phosphate and uridine 5′ phosphate 6-aminohexyl esters (Smrt, Coll. Czech. Chem. Commun., 44:589, 1979).

In still other embodiments the partial metal chelator synthons are derivatives of synthons normally employed to prepare peptoids, PNA (see Buchardt et al., PCT WO 92/20702 and Buchardt et al., U.S. Pat. No. 5,719,262), morpholinos (see Summerton 1730 and Weller, U.S. Pat. No. 5,698,685; Summerton et al., U.S. Pat. No. 5,378,841 and Summerton and Weller, U.S. Pat. No. 5,185,444), PENAMs (see Shah et al., U.S. Pat. No. 5,698,685), and nucleobase polymers with linkages comprising carbamate (see Stirchak and Summerton, J. Org. Chem. 52:4202, 1987), amide (see Lebreton et al., Synlett. February 1994:137), methylhydroxylamine (see Vasseur et al., J. Am. Chem. Soc. 114:4006, 1992), 3′-thioformacetal (see Jones et al., J. Org. Chem. 58:2983, 1993), sulfamate (see Huie and Trainor, U.S. Pat. No. 5,470,967) and others (see Swaminathan et al., U.S. Pat. No. 5,817,781 and Freier and Altmann, Nucl. Acids Res. 25:4429, 1997, and references cited therein). In other embodiments, the partial metal chelator synthons employ ‘native chemical ligation’ or ‘click chemistry’ to allow their 1740 facile condensation with an unprotected binding element or other unprotected synthons (Yeo et al., Chemistry, 10(19):4664, 2004; Kold and Sharpless, Drug Discov. Today, 8(24), 1128, 2003 and references therein). Methods for preparing these partial metal chelator synthons based on derivatives of the above synthons and chemistries are known to those in the art.

In embodiments where the partial metal chelator is attached to a nucleobase or sugar in a phosphoramidite or H-phosphonate synthon, such synthons allow the facile placement of one or both of the partial metal chelators within the binding element while preserving the continuity and binding fidelity of the binding element. Although in some probe embodiments this may be desirable (e.g. in siRNA where free ends are preferable for engaging the probe with the RISC complex), in general it is more desirable to not place any partial metal chelator in the binding element, thereby simplifying the design, construction and use of the probe. In these embodiments where the partial metal chelators are on opposing ends of the probe and outside the binding element, it is particularly desirable that the partial metal chelator synthons not contain features of the binding element (e.g. nucleobases) in order to avoid unintended and non-specific interactions with the target. In these embodiments, it is desired that the synthons provide partial metal chelators with only the minimum of structure to allow the partial metal chelators in the final probe to form the coordination complex with one another but bind to nothing else. As such, still other partial metal chelate synthons are even more preferred in the assembly of probes having partial metal chelators on ends of the probe that lie outside the binding element.

Thus, the tethered partial metal chelator is attached to the 3′ or 5′ terminal phosphate of polynucleotides and nucleic acids using synthons that provide a phosphoamidate linkage, e.g. partial metal chelators bearing a free amine such as an N-alkylamine IDA (Chu et. al., Nucl. Acids Res., 11:6513, 1983).

In embodiments where the binding element is PNA, peptide or other polyamide, preferred partial metal chelator synthons for attaching the partial metal chelators outside of the binding element are amino acid derivatives as those noted above. In embodiments where the binding element is a nucleobase polymer assembled from phosphoramidites or H-phosphonates, preferred partial metal chelator synthons include those partial metal chelator phosphoramidites already noted above. Other preferred partial metal chelator synthons include the following:

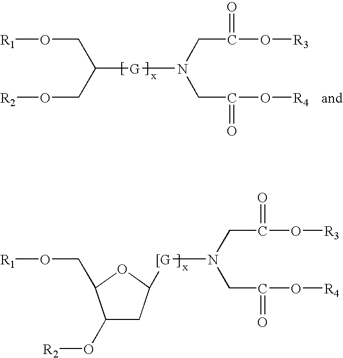

where R₁ is a hydroxyl protecting group (e.g. DMT and other trityl derivatives well known in the art); R₂ is selected from the group consisting of a linker;

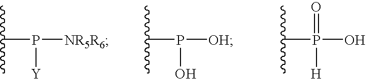

and salts thereof, R₃ and R₄ are carboxyl protecting groups; R₅ and R₆ are independently selected from the group consisting of C₃₋₁₀ branched alkyl and C₁₋₁₂ unbranched alkyl, and cyclic hydrocarbons; Y is a beta-cyanoethyl group; and G is selected from the group consisting of alkyl, heteroalkyl, aryl, aryl(alkylene), heteroaryl, heteroaryl(alkylene), carbocycle, carbocyle(alkylene), heterocycle, heterocycle(alkylene), and

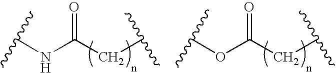

where n=1 to 10; and X is from 0 to 10. For example, one embodiment of a preferred partial metal chelator synthon according to this preferred class is:

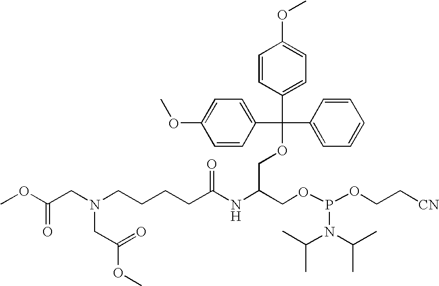

Preferred partial metal chelator synthons are readily prepared by reacting bromo-ethylacetate with 2-aminoalkyl-1,3-propanediol derivatives, and converting these to the DMT-protected phosphoramidites or H-phosphonates using previously described methods (Nelson, Kent and Muthini, Nucleic Acids Res., 20(23):6253, 1992 and Sinha and Cook, Nucleic Acids Res., 16(6):2659, 1988). Other methods for preparation have already been noted above. Alternative protecting groups to DMT will be well known to those skilled in the art (e.g. T. W. Greene and P. G. M. Wuts, Protective Groups in Organic Synthesis, 3^{rd} Edition, John Wiley & Sons, New York, 1999; Beaucage and Iyer, Tetrahedron, 48(12):2223, 1992; and Beaucage and Iyer, Tetrahedron, 49(12):6123, 1993). Preferably, the DMT-protected phosphoramidites or H-phosphonate derivatives employ carboxyl-protecting groups (i.e. R₃ and R₄ in the structures above) such as benzyl ester, 9-fluorenylmethyl ester, or N-phthalimidomethyl ester, which can be removed under ultra-mild alkaline conditions (e.g. 0.05 M potassium carbonate in anhydrous methanol for 4-24 hours). Other methods for preparing partial metal chelator synthons of phosphoramidites or H-phosphonates are apparent to those skilled in the art.

In other embodiments, optionally protected partial metal chelator synthons are also prepared bearing moieties that react in-solution or on solid-phase with a suitably modified binding element, but that do not self-react with the unprotected synthon. For example, unprotected and protected partial metal chelator synthons can be reacted with a binding element as described below in “Probe Assembly Methods”, through reactive moieties such as thiol, maleimide, amine, dianhydride and alkyl halide (see Andres et al., Eur. J. Org. Chem., page 3769, 2003; Hochuli et al., J. Chromatography, 411:177, 1987; Kroger et al., Biosensors & Bioelectronics, 14:155, 1999; Sigal et al., Anal. Chem. 68:490, 1996; Chen et al., J. Am. Chem. Soc., 124:3840, 2002; Chen et al., Biochemica et Biophysica Acta, 1697:39, 2004; and U.S. Pat. Nos. 4,479,930; 5,618,513 and 5,736,120). Unprotected partial metal chelator synthons bearing azide, alkyne, cysteine with a free α-amine, or thioester moieties are particularly preferred since they can be prepared and efficiently coupled to a suitably modified but unprotected binding element (e.g. a peptide, nucleobase polymer, or other species) in a solution-phase or solid-phase reaction using ‘native chemical ligation’ or ‘click chemistry’ using methods generally described in Yeo et al., Chemistry, 10(19):4664, 2004; Kold and Sharpless, Drug Discov. Today, 8(24), 1128, 2003 and references therein.

### Linker and Spacer Synthons

Synthons suitable for assembly of a linker or a spacer are obtained from a group that includes, but is not limited to, natural amino acids, unnatural amino acids, diols, diamines, haloalkyl acids, polyoxyethylene derivatives, and those compounds disclosed in the Novabiochem 2003/04 Catalog, Novabiochem, San Diego, Calif.; Gammill et al., U.S. Pat. No. 5,304,548; Konishi et al., U.S. Pat. No. 6,127,337; Desk Reference of Functional Polymers Synthesis and Applications, edited by Reza Arshady, (1997), American Chemical Society, Washington, D.C.; and the references disclosed therein. Other compounds useful for providing synthons for assembly of the linker or spacer include;

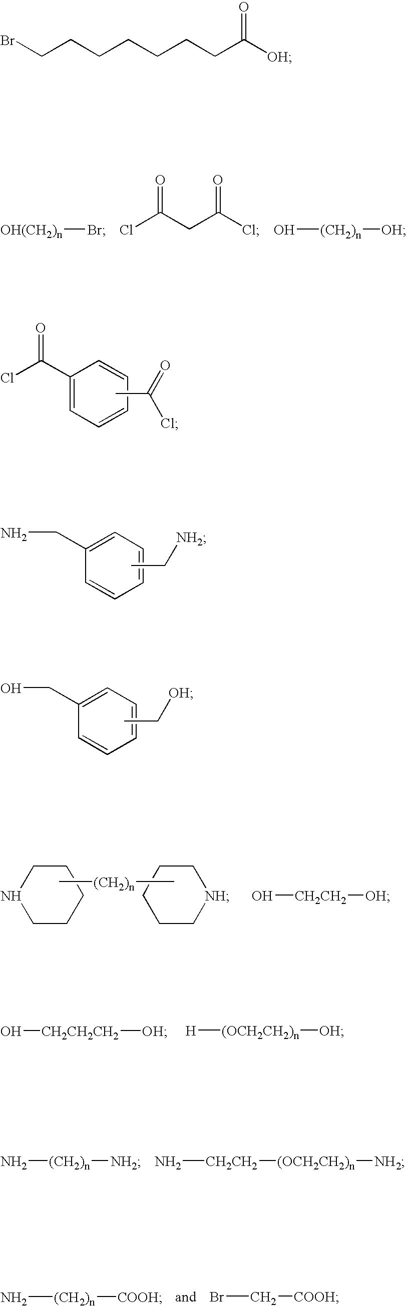

where n=1 to about 20.

### Label Synthons

The manner in which the label moieties are covalently attached to the probe backbone depends upon the nature of the backbone. In the case of peptides or peptide nucleic acid, amino acid derivatives for the incorporation of DABCYL, EDANS, and TMR are available (Nova Biochem). These are incorporated into a peptide or peptide nucleic acid backbone using methodologies known to those skilled in the art of solid phase synthesis.

A wide range of other fluorophore and quencher moieties are also available as amine-reactive or thiol-reactive derivatives (Molecular Probes, Eugene Oreg.). By incorporating standard amino acids, such as lysine residues or cysteine residues, these labels may also be incorporated into a probe (see Examples 6.2.5 and 6.2.6).

Attachment of these label moieties to DNA is somewhat more complicated. Presently, fluorophore and quencher moieties (including DABCYL) are commercially available (Integrated DNA Technologies, and elsewhere) as phosphoramadite derivatives for simple incorporation into DNA sequences by standard solid phase synthesis techniques. However, use of these derivatives may prevent the inclusion of terminal primary amines and thiols in the DNA oligomer, which are necessary for conjugating chelating groups to the probe. Moreover, the positioning of the label may be too distal from the chelating group that is added in a separate step. In the absence of a phosphoramadite derivative that possesses both the chelating group and the label moiety, the preferred means of constructing a labeled DNA probe is to prepare two terminal conjugating groups. Each group (which can be constructed from modified amino acids) contains both a chelating group and a label moiety. One of these groups is prepared as a thiol-reactive derivative and the other is prepared as an amine-reactive derivative. In a solution-phase reaction, the derivatized chelator/label can then be conjugated to a modified DNA sequence bearing a thiol at one terminus and an amine at the other (see Example 6.1.4). Techniques for the solution-phase conjugation of DNA modified with an amine and/or a thiol are known to one skilled in the art.

For example, a short peptide sequence is prepared by standard solid-phase methods which possesses an IDA chelating group conjugated to a lysine residue, a fluorophore conjugated to a cysteine residue, and a terminal group which bears a thiol-reactive maleimide. This short sequence is then coupled to a terminal thiol of a modified DNA sequence by standard solution-phase methods. In a separate solid-phase reaction, a second short peptide sequence is prepared which possesses an IDA chelating group conjugated to a lysine residue, a DABCYL moiety conjugated to a second lysine residue, and a terminal group which bears an amine-reactive N-hydroxysuccinimide ester. This short sequence is then coupled to the opposite terminus of the modified DNA sequence, which bears a terminal amine. In this fashion, a DNA probe is constructed which possesses two chelating groups and an interactive label pair.

### Probe Assembly Methods

The probes are synthesized by a suitable strategy such as by classical solution synthesis, exclusively solid-phase synthesis, or a combination of both strategies. According to preferred embodiments of a solid-phase synthesis strategy, the probes are prepared by the uni-directional assembly of suitably protected synthons off a resin-bound synthon (see Stewart et al., Solid Phase Peptide Synthesis, 2^{nd} Edition, Pierce Chemical Co., Rockford, Ill., 1984; Bayer & Rapp, Chem. Pept. Prot. 3:3, 1986; Atherton, et al. Solid Phase Peptide Synthesis: A Practical Approach, IRL Press, Oxford, 1989; Barany and Merrifield, Solid-Phase Peptide Synthesis, pp. 3-284 in The Peptides: Analysis, Synthesis, Biology. Vol. 2: Special Methods in Peptide Synthesis, Part A; Merrifield, et al. J. Am. Chem. Soc. 85:2149, 1963; the Novabiochem 2003/04 Catalog, Novabiochem, San Diego, Calif.; Dorman, Noble, McBride, & Caruthers Tetrahedron 40:95, 1984; Adams, Kavka, Wykes, Holder, & Galluppi, J. Am. Chem. Soc. 105:661, 1983; Matteucci, & Caruthers, J. Am. Chem. Soc. 103:3185, 1981; Beaucage & Caruthers Tetrahedron Lett. 22:1859, 1981; Stawinski and Stromberg, Methods Mol. Biol., 288:81, 2005; and the 2006 Glen Research Catalog, Glen Research, Sterling, Va.). Where synthons bear two or more reactive moieties, all but one reactive moiety are typically protected with suitable protecting groups to prevent undesired chemical reactions from occurring during the assembly of synthons off the synthon. After a first synthon is attached to the resin-bound synthon, one additional protecting group is selectively removed from each of the first synthons to allow subsequent couplings with other synthons. The conditions for the removal of this protecting group do not remove the other protecting groups that may exist elsewhere in the evolving resin-bound structure. Two protecting groups that are removed by conditions that are different from one another are said to be ‘orthogonal’ protecting groups.

In some embodiments, selective removal of a protecting group from the most recently coupled synthon is not required to couple a subsequent synthon as would occur, for example, with a bifunctional synthon such as a diester, diamine, dialcohol, etc. The process of coupling synthons is repeated until the last synthon is coupled. The last synthon will, in some embodiments, not have any protecting groups.

The synthon protecting groups not participating in the assembly process must remain intact during coupling (e.g. the protecting groups on side-chains and reactive exocyclic nucleobase groups). However, such protecting groups are removable upon the completion of synthesis, using reaction conditions that will not alter the finished probe. In Fmoc chemistry, such protecting groups are mostly t-butyl (tBu) or trityl based. In Fmoc chemistry, the preferred protecting groups are 2,2,5,7,8-pentamethylchroman-6-sulfonyl (Pmc) or 2,2,4,6,7-pentamethyldihydrobenzofuran-5-sulfonyl (Pbf) for Arg; trityl for Asn, Cys, Gln and His; tert-butyl for Asp, Glu, Ser, Thr and Tyr; and tBoc for Lys and Trp. In phosphoramidite chemistry, the preferred protecting groups are benzoyl for dC/C and dA/A, isobutyrl and dimethylformamide for dG/G, and beta-cyanoethyl for the phosphite triester and phosphotriester linkage intermediates. More preferred protecting groups for phosphoramidite chemistry are those removed under ultra-mild conditions (e.g. phenoxyacetyl or isobutryl for dA/A, 4-isopropyl-phenoxyacetyl (iPr-Pac) for dG/G, and acetyl for dC/C). In H-phosphonate chemistry, preferred exocyclic protecting groups are the same as those in phosphoramidite chemistry. In some embodiments of phosphoramidite and H-phosphonate coupling chemistry, exocyclic protecting groups are optional (Gryaznov and Letsinger, J. Am. Chem. Soc., 113:5876, 1991; and Kung and Jone, Tet. Lett., 33(40):5869, 1992).

In some embodiments, synthons are condensed on the solid-phase without modification with protecting groups. For example, synthons may be condensed without protecting groups if they have only one reactive moiety, or by using ‘native chemical ligation’ or ‘click chemistry’ to allow condensation between an unprotected synthon and a protected or unprotected resin-bound species (Yeo et al., Chemistry, 10(19):4664, 2004; Kold and Sharpless, Drug Discov. Today, 8(24), 1128, 2003 and references therein). In 1960 still other embodiments, an activator may or may not be required (e.g. if the synthon itself is inherently reactive, as is often the case for an ester or alkyl halide).

Solid-phase coupling of amino acid synthons is carried out on their carboxy-terminus by first activating and coupling the amino-protected synthon to a suitable solid support (typically a polystyrene support or a derivative thereof). An ester linkage is formed on the end of the amino acid synthon attached to the resin when the first coupling is made to a chloromethyl, chlorotrityl or hydroxymethyl linker resin, and the resulting probe has a free carboxyl group on one end. Alternatively, when an amide resin such as benzhydrylamine, Rink amide or PAL resin is used, an amide bond is formed and the resulting probe has a free carboxamide group on one end. These resins, whether polystyrene- or polyamide-based or polyethylene glycol-grafted, are commercially available, and their preparations have been described (see Stewart et al., Solid Phase Peptide Synthesis, 2^{nd} Edition, Pierce Chemical Co., Rockford, Ill., 1984; Bayer & Rapp, Chem. Pept. Prot. 3:3, 1986; and Atherton, et al. Solid Phase Peptide Synthesis: A Practical Approach, IRL Press, Oxford, 1989).

For example, the protected amino acid synthon is attached to a hydroxyethyl resin using various activating agents including dicyclohexylcarbodiimide (DCC), N,N′-diisopropylcarbodiimide (DIPCDI) and carbonyldiimidazole (CDI). The protected amino acid synthon is also attached to chloromethyl or chlorotrityl resin directly in its cesium tetramethylammonium salt form or in the presence of triethylamine (TEA) or diisopropylethylamine (DIPEA). Attachment to an amide resin employs the same method as for amide bond formation during synthon coupling reactions. Following the attachment of the amino acid synthon to the resin support, the amino protecting groups are removed using various reagents depending on the protecting chemistry (e.g. TFA for BOC, or piperidine for Fmoc). The extent of Fmoc removal can be monitored at 300-320 nm. After removal of the amino protecting group, the remaining protected synthons are coupled stepwise in the required order to obtain the desired resin-bound probe.

Each protected amino acid synthon is used in excess (e.g. >2.0 equivalents), and the couplings are often carried out in NMP (“N-methylpyrrolidone”) or in DMF (“N,N-dimethylformamide”), CH₂Cl₂ or mixtures thereof. The extent of completion of the coupling reaction is monitored at each stage, e.g., by the ninhydrin reaction (see Kaiser et al., Anal. Biochem. 34:595, 1970). In cases where incomplete coupling is found, the coupling reaction is extended and repeated and may have chaotropic salts added. The coupling reactions can be performed automatically with commercially available instruments. After the entire assembly of the desired probe, the resin-bound probe is cleaved with a reagent with proper scavengers (e.g. HF for BOC chemistry and TFA for Fmoc chemistry, together with the appropriate scavengers).

In alternative embodiments of probe assembly, solid-phase coupling of phosphoramidite or H-phosphonate synthons is carried out by first coupling a 5′-dimethoxytrityl (DMT)-protected synthon to a suitable solid support (e.g. a controlled pore glass support) bearing a free hydroxyl, wherein the free hydroxyl is generated subsequent to removal of a DMT protecting group by treatment with an acid such as dichloroacetic acid (DCA) or trichloroacetic acid (TCA). Routinely, supports are prepared wherein the free hydroxyl is from a first pre-attached synthon attached via a succinate linkage. Alternatively, the first pre-attached synthon is attached via the more rapidly cleavable Q-linker based on hydroquinone-O,O′-diacetic acid (Pon and Yu, Tetrahedron Lett., 38:3327, 1997). In preferred embodiments, no synthon is pre-attached and the first free hydroxyl is provided by a DMT-protected ‘universal support’ after treatment with acid (Scott et al., Innovations and Persepective in Solid Phase Synthesis, 3^{rd} International Symposium, pages 115-124, 1994; Azhayev, Tetrahedron, 55:787, 1999 and Azhayev and Antopolsky, Tetrahedron, 57:4977, 2001). Whether the free hydroxyl is provided by a first pre-attached synthon or from a universal support, the first coupling of the 5′ trityl-protected synthon requires activation of the phosophoramidite or H-phosphonate with activators known to those in the art (e.g. tetrazole; 4,5-dicyanoimidazole; 5-ethylthio-1H-tetrazole; and 5-benzylthio-1H-tetrazole activators for phosphoramidites and pivaloyl chloride and adamantoyl chloride for H-phosphonates). Optional capping is performed in phosphoramidite chemistry using, for example, acetic anhydride. In H-phosphonate chemistry the activator performs a dual role as both activator and capping reagent, although an optional additional capping reagent such as isopropyl phosphite may also be employed.

Subsequent to coupling and optional capping, the phosphite triester linkage is oxidized to the phosphotriester linkage in phosphoramidite chemistry using any of a variety of aqueous and non-aqueous oxidizing agents known in the art, such as iodine in THF/Pyridine/H₂O, anhydrous t-butyl hydroperoxide, or anhydrous 10-camphorsulfonyl-oxaziridine (Hayakawa, et al., J. Am. Chem. Soc., 112:1691, 1990; Sproat, et al., Nucleosides & Nucleotides, 14:255, 1995 and the 2006 Glen Research Catalog, Glen Research, Sterling, Va.). In contrast, oxidation of the H-phosphonate diester linkages in H-phosphonate chemistry is performed after all synthons have been coupled using, for example iodine in THF/pyridine/water followed by TEA/water/THF, or iodine in THF followed by N-methylmorpholine in THF/water (Stawinski and Stromberg, Methods Mol. Biol., 288:81, 2005 and the 2006 Glen Research Catalog, Glen Research, Sterling, Va.).

Following the steps of coupling of phosphoramidite or H-phosphonate synthons, optional capping, and oxidation in the case of phosphoramidites, the 5′ DMT protecting groups of the coupled synthon are then removed using a weak acid such as DCA or TCA. The extent of DMT removal is monitored spectrophotometrically. After removal of the DMT protecting group, the remaining protected synthons are coupled stepwise using repetitions of the above steps in the required order to obtain the desired resin-bound protected probe. In the case of resin-bound protected probes prepared by H-phosphonate chemistry, oxidation is performed subsequent to all synthon couplings as described above. Deprotection and cleavage of the oxidized resin-bound protected probes is then accomplished using a variety of strongly alkaline deprotection and cleavage reagents known to those in the art, such as concentrated ammonium hydroxide at 55° C. for 24 hours, or a 50:50 mixture of aqueous ammonium hydroxide and aqueous methylamine (i.e. ‘AMA’) at 65° C. for 5 min to cleave the probe from the support followed by 65° C. for 5 min, 55° C. for 10 min, 37° C. for 30 min, or 25° C. for 90 min to deprotect the probe. In preferred embodiments where synthons are employed in the probe that would be sensitive to these harsh alkaline conditions (e.g. TAMRA, Cy5, TET, and HEX labels), ultra-mild deprotection and cleavage is employed using concentrated ammonia at room temperature for 4 hours or more preferably, 0.05 M potassium carbonate in anhydrous methanol for 4-24 hours (2006 Glen Research Catalog, Glen Research, Sterling, Va.).

In still other embodiments of probe assembly, all or some of the synthon condensations are performed in-solution to provide the final probe, rather than all being performed on the solid-phase (U.S. Pat. No. 5,200,504). For example, binding elements (e.g. DNA, RNA, PNA and peptides) bearing primary amines or thiols at either end can be purchased commercially (Integrated DNA Technologies, Applied Biosystems, and elsewhere). Synthons bearing partial metal chelators, labels, spacers and combinations thereof are prepared with functional groups reactive toward these amines or thiols. In single or sequential solution-phase reactions, the synthons are then conjugated to the primary amines and thiols of the binding element. In particular, techniques for the solution-phase conjugation of synthons with DNA, RNA, PNA and peptides modified with an amine and/or a thiol are known in the art. In another embodiment of solution-phase probe assembly, unprotected synthons are coupled to a suitably modified but unprotected binding element in a solution-phase reaction using ‘native chemical ligation’ or ‘click chemistry’ (Yeo et al., Chemistry, 10(19):4664, 2004; Kold and Sharpless, Drug Discov. Today, 8(24), 1128, 2003 and references therein). Other embodiments for the solution-phase assembly of the probes of the disclosure will be apparent to one skilled in the art.

Although it is preferred that synthons are incorporated into the probe chemically using known solution-phase or solid-phase synthesis methodologies, alternatively, enzymatic procedures for incorporating synthons could be used. For example, the partial metal chelator may be attached to a phosphate group or nucleobase in a deoxynucleoside triphosphate (dNTP). An enzyme, such as the Klenow fragment of DNA polymerase I or any of a variety of thermophilic polymerases, is then used to incorporate (according to Watson-Crick pairing rules with a template sequence) the dNTP bearing the partial metal chelator along with other dNTPs into a deoxyribonucleotide polymer. The synthesized polymer forms part of a duplex, which is then denatured to yield a single-stranded polymer capable of functioning as a probe.

In a more preferred embodiment, the enzymatic attachment or detachment of a partial metal chelator is part of an assay to generate the probe in situ and detect a target. For example, a peptide binding element bearing a single partial metal chelator is a substrate for a target kinase. The target kinase utilizes ATP to attach a phosphoryl group (i.e. the second partial metal chelator) to the peptide binding element and form the coordination complex in situ (see section “5.8 Methods of Use and Other Embodiments”).

Final assembled probes, whether assembled by Fmoc, BOC, phosphoramidite, H-phosphonate, or other chemistries, are then combined with a metal ion as described more fully in section “5.6 Transition Metals”, vide supra.

### 5.8 Methods of Use And Other Embodiments

Several probe embodiments are illustrated interacting with a variety of target types in FIGS. 1A, 1B and 1C. In other embodiments, target molecules are also covalently or non-covalently modify a given probe after binding by the probe, and vice versa.

The present disclosure includes kits containing reactive chelator synthons or combination chelator/label synthons and instructions to use these synthons to prepare probes according to the present disclosure for the detection of a variety of analytical targets chosen by the user. The methodologies for conjugating these reactive synthons to a target binding element includes solid-phase or solution-phase techniques. The reactive chelator synthons comprise a chelation site such as IDA and an amine- or thiol-reactive group such as an N-hydroxysuccinimide ester or maleimide. A combination chelator/label synthon further contains, in proximity to the chelator, a label moiety such as a fluorophore or quencher. A kit optionally includes one or more reactive spacer synthons. A kit may also include salts of appropriate transition metals, in solid or preferably solution form.

Reactive chelator/label synthons comprising IDA and one member of an interactive probe are preferred. Such synthons are then matched with a second such synthon comprising an appropriate label to pair with the first, along with a second IDA. A probe constructed from these two synthons possesses an IDA/IDA chelator pair and an interactive label pair. For example, a kit includes a reactive IDA/DABCYL synthon for one of the conjugation sites, and a reactive IDA/fluorescein synthon for the second site. In some embodiments these two sites are the two termini of the binding element, but this need not be the case.

In an alternative embodiment, such a kit contains a reactive NTA/DABCYL synthon for one of the conjugation sites, and several versions of a reactive polyhistidine/fluorescein synthon for the second conjugation site. These different versions vary in the length of the polyhistidine sequence (from about 2 to about 6 histidine residues). Probes constructed with longer polyhistidine sequences exhibit a greater transition metal binding energy, and therefore a stronger force for holding the probe in the closed conformation, than probes with short polyhistidine sequences. A kit with several of these variable polyhistidine synthon to choose from allows a researcher to choose the one best suited for the individual application.

Target detection methods may be qualitative or quantitative, but do not require washing to remove unbound probes, if interactive labels are used. A method comprises mixing a probe backbone with a transition metal solution, thus forming a constrained cyclized probe which is then added to a sample suspected to contain target analyte and ascertaining whether or not a detectable signal occurs. Homogeneous methods are preferred, although probes with interactive labels according to this disclosure may be used in heterogeneous binding methods. A control without target analyte may be run simultaneously, in which case signal generation of the sample and the control may be compared, either qualitatively or quantitatively by measuring the two and calculating a difference. Methods include real-time and end-point detection of products of nucleic acid synthesis reactions, such as transcription, replication, polymerase chain reaction (PCR), self-sustained sequence reaction (3SR), strand-displacement amplification reaction (SDA), and Q-beta replicase-mediated amplification reaction. If the probe has a non-interactive label, separation of bound from unbound probes is included in the method.

Quantitative methods can employ quantification techniques known in the art. An end point for a sample may be compared to end points of a target dilution series, for example. Also, readings may be taken over time and compared to readings of a positive or negative control, or both, or compared to curves of one or more members of a target dilution series.

Methods employing probes with interactive labels include in situ detection, qualitative or quantitative, of biopolymers in, for example, fixed tissues without destruction of the tissue. Because a large excess of probes can be used without the need for washing and without generation of a large background signal, in situ methods of this disclosure are particularly useful. In situ binding methods, according to this disclosure include ‘chromosome painting,’ for the purposes of mapping chromosomes, and for detecting chromosomal abnormalities (Lichter et al., Proc. Natl. Acad. Sci. U.S.A., 87:6634, 1990).

Methods employing probes with interactive labels include in vivo methods. A large excess of probes can be used without the need to wash. Probes are useful as “vital stains” (agents that can stain specific constituents of cells without killing them) in methods for the detection of targets in vivo. They can be used in methods to locate specific nucleic acids within various living cells or organelles within living cells. They can be used in methods to identify specific cell types within a tissue or within a living organism. In other methods probes can be delivered to the interior of cells via known techniques, e.g., by liposomes or by making the cell membranes porous to macromolecules using electroporation. In preferred embodiments, the probe is modified with any of a variety of transduction domains to permit spontaneous entry into cells as reviewed by Dietz and Bahr, and incorporated herein by reference (Dietz and Bahr, Mol. Cell. Neurosci., 27:85, 2004).

By using multiple probes with interactive labels that generate different, non-interfering detectable signals, e.g., fluorescence at different wavelengths or fluorescence and colored product formation, methods of this disclosure can detect multiple targets in a single method. Also, multiple probes, each specific for different regions of the same target, yet having the same label pair, can be used in order to enhance the signal. If multiple probes are used for the same target, they should bind to the target such that neighboring probes do not quench each other.

Certain embodiments of methods comprise addition of probes with interactive labels according to this disclosure to a sample and visualization with the naked eye for detection of a specific target in a complex mixture. By comparison with positive standards or the results obtained with positive standards, visualization can be roughly quantitative.

Preferred probes with interactive labels emit a high level of positive signal only in the target-bound, open conformation and little-to-no signal in the closed conformation. Furthermore, preferred probes do not assume the open conformation unless bound to target, remaining closed when non-specifically bound. As described above, this leads to a background (i.e. non-specific) signal that is very low. Therefore, the use of these probes greatly simplifies conventional, heterogeneous methods because either no washing is required or only mild, low stringency washing is used to further reduce any background signal present after binding.

Heterogeneous methods may include the use of capture probes. In a capture-probe method, a capture probe is attached to a surface either before or after capturing a target. Surface attachment means and steps are well known in the art and include reaction of biotin-labeled capture probes to avidin-coated surfaces, for example, magnetic beads. The capture probe includes a target binding element which binds to the target to capture it. Probes having a target binding element that binds to the target at a location other than the location where the capture probe binds, may be added before or after or at the same time as the capture probes and before or after washing the capture probe-target complexes. If a probe with interactive label moieties is used, washing is not required, although mild washing may enhance the method result by lowering background. If a probe with a non-interactive label is added, then a surface bearing the capture probe-target-probe complexes should be washed as in a typical heterogeneous method.

Probes open very quickly upon interaction with a target. The ability to interact is concentration dependent, as workers skilled in intermolecular binding methods recognize. Method conditions may be selected in which the probes with interactive labels respond to the presence of target and generate signal very quickly. Because of this, methods include real-time monitoring of production of specific targets. Nucleic acid synthesis processes such as transcription, replication or amplification can be monitored by including probes in the reaction mixture and continuously or intermittently measuring the fluorescence. Probes are used in substantial excess so that the relatively abundant probes find their targets in nascent nucleic acid strands before the targets are sequestered by the binding of complementary strands.

The use of probes with interactive labels in methods for the identification of products of nucleic acid amplification reactions generally eliminates the need for post-amplification analysis to detect desired products and distinguish desired products from unwanted side reactions or background products. Of course, probes according to the disclosure can be added at the end of a synthesis process for end-point detection of products. In methods for monitoring the progress of an amplification reaction, the probes can be present during synthesis. The presence of probes improves the accuracy, precision and dynamic range of the estimates of the target nucleic acid concentration. Reactions in closed tubes may be monitored without ever opening the tubes. Therefore, methods using these probes with interactive labels can limit the number of false positives, because contamination can be limited.

Preferred methods utilize multiple probes with interactive labels linked to a solid surface or surfaces (e.g. in a microarray). Because probes having interactive labels are used, washing is not required to measure target binding-induced signal. Probes attached to a solid surface are referred to herein as ‘tethered probes.’ Attachment may be either covalent or noncovalent, with covalent being preferred Any type of surface may be used, including beads, glass slides, membranes, microtiter wells, and dipsticks. Preferred surfaces are neutral with respect to the components of the probe; that is, surfaces that do not interact with the binding element, do not interact with the label moieties and do not interfere with the probe signal. An example of such a surface is a surface coated with a siliconizing agent. In preferred embodiments, the surface on which the detection method is performed is the same surface on which the probe was prepared by solid-phase synthesis methods (e.g. as by in situ microarray synthesis using ink-jet or photoactivatable monomer methodologies).

A preferred surface does not significantly interfere with: a) the ability of the metal-mediated coordination complex of the probe to maintain the probe in the closed conformation; b) the binding of the binding element to the target; c) the separation of the partial chelators in the open conformation; d) the interaction of proximate label moieties in the closed conformation; e) the quenching of a luminescent moiety by a quenching moiety; and f) the luminescent properties labels in the open conformation. In order to accomplish these goals, the surface of a solid-support may be modified with any of a variety of reagents that change the surface properties of a support to make it suitable for probe-target binding and maintaining the functionality of an interactive label pair (for example, U.S. Pat. No. 6,951,682; Peterson, et al. Nucleic Acids Res, 29(24):5163, 2001; Urakawa, H., et al., Appl. Environ. Microbiol., 69(5):2848, 2003 and the references therein; and the reagents available from Gelest, Inc., Tullytown, Pa.). Additionally, the insertion of the spacers and linkers between the modified or unmodified substrate is a method well known in the art to accomplish the required goals above in order to properly present tethered probes to target.

Tethered probes are advantageously useful in methods for the simultaneous determination of a predetermined set of targets (e.g. a microarray of tethered probes having interactive labels for use in expression profiling of a known mixture of RNA targets). For example, a series of luminescent probes can be prepared, each comprising a different target-binding element. Each probe may be linked to the same support surface at its own predetermined location. After contacting the support and the sample, the support may be stimulated with light of an appropriate frequency. Luminescence will occur at those locations where tethered probes have bound with target molecules from the sample.

Additional embodiments of methods utilize probes of the disclosure in RNA interference (i.e. RNAi) pathways. RNAi is a normal defense pathway in plant and animal cells that destroys mRNAs that might be harmful to a cell (e.g. inappropriately expressed viral nucleic acids). It is known in the art that ‘short double-stranded interfering RNA’ (siRNA) that is homologous to a target mRNA can be introduced inside a cell to coopt the RNAi machinery to destroy the desired target mRNA. When an siRNA is inserted into a cell, the siRNA duplex is unwound, and the antisense (i.e. guide) strand of the duplex is loaded into an assembly of proteins to form the RNA-induced silencing complex (RISC) that is responsible for identifying mRNAs that are closely complementary to the siRNA antisense strand.

When a matched mRNA finally docks onto the siRNA, an enzyme know as slicer cuts the captured mRNA strand in two. The RISC then releases the two pieces of the mRNA (now rendered incapable of directing protein synthesis), and catalytically repeats this cleavage on other mRNA substrates.

A major shortcoming of siRNA is off-target transcript silencing that limits its specificity and utility in functional genomic and therapeutic applications (Jackson et al., Nature Biotechnol., 21:635, 2003). Off-target transcript silencing is widespread and mediated largely by limited target sequence complementarity to the seed region of the siRNA guide strand (Jackson et al., RNA, 12:1179, 2006). Thus, a key advantage of using the probes disclosed herein is their enhanced specificity and reduced off-target transcript silencing in RNAi applications.

The structural requirements for effective RNAi by an siRNA duplex has been studied in a variety of systems, revealing many of the key requirements for siRNA length, structure, chemical composition, and sequence that are essential to mediate efficient RNAi activity (see Allerson et al., J. Med. Chem., 48:901, 2005; Amarzguioui et al., Nucleic Acids Res., 31:589, 2003; Chiu and Rana, RNA, 9:1034, 2003; Czauderna et al., Nucleic Acids Res., 31:2705, 2003; Elbashir et al., EMBO J., 20:6877, 2001; Jackson et al., RNA, 12:1197, 2006; and Kraynack and Baker, RNA, 12:163, 2006). These studies have shown that 21-nucleotide siRNA duplexes are most active when containing 3′-terminal dinucleotide overhangs. Furthermore, complete substitution of one or both siRNA strands with 2′-deoxy(2′-H) or 2′-O-methyl nucleotides abolishes RNAi activity, whereas substitution of the 3′-terminal siRNA overhang nucleotides with 2′-deoxy nucleotides (2′-H) was shown to be tolerated. In contrast, siRNA with alternating 2′-O-methyl and 2′-O-fluoro nucleotides had activity equivalent to unmodified duplexes. Single mismatch sequences in the center of the siRNA duplex were shown to abolish RNAi activity. In addition, these studies also indicate that the position of the cleavage site in the target RNA is defined by the 5′-end of the siRNA guide sequence rather than the 3′-end of the guide sequence. Other studies have indicated that a 5′-phosphate on the antisense strand of a siRNA duplex is required for siRNA activity and that ATP is utilized to maintain the 5′-phosphate moiety on the siRNA (see Nykanen et al., Cell, 107:309, 2001).

There is disclosed a method comprising introducing into the intracellular space probes of the disclosure wherein the flexible binding element is a mRNA. Preferably, the flexible binding element is selected from the group consisting of miRNA, siRNA, shRNA, and long dsRNA. Methods for introducing chemically modified RNA suitable for engaging in RNA interferences are known (Jackson et al., RNA, 12:1197, 2006). Preferably, the linkage of the partial metal chelator to the miRNA, siRNA, shRNA, or long dsRNA does not interfere with the ability of these molecules to interact with target RNA and the RISC complex, particularly when located internal to the termini. Without being bound limited by theory, probes wherein the binding element is RNA allows for increased specificity for the target mRNA as perfect pairing of the probe to the mRNA target is needed to disrupt the metal-mediated coordination complex.

Method kits include at least one probe and instructions for performing a target detection method. The probe may already be in a binary state (complexed with a transition metal), or it may include separate transition metal solutions and a probe backbone with instructions for mixing two components to prepare the binary probe complex prior to use. Kits may optionally include method reagents, e.g. salts (including transition metal salts), buffers, and denaturants. Kits may optionally include a target or model target for a positive control test, and a target-less ‘sample’ for a negative control test.

Nucleic acid amplification method kits include, in addition to some or all of the above, primers, nucleotides, polymerases and polymerase templates for the assay and for control assays. Vital stain kits may include, in addition to probe and instructions, permeabilizing agents, liposome precursors, buffers, salts, counter stains and optical filters. In situ kits may include, in addition to probe and instructions, fixatives, dehydrating agents, proteases, counter stains, detergents, optical filters and coated microscope slides.

Field kits may include, in addition to instructions, tethered probes with interactive labels. At least one probe may be tethered to beads, slides, wells or a dipstick. Multiple probes may be included, including a positive control probe that will bind to a component of uninfected samples. Field kits may include, in addition to instructions, untethered probes. Such kits are designed, for example, to detect infectious agents or genes. Kits for gene detection may include negative and, sometimes, positive targets for use as method controls.

While the above description contains many specificities, these should not be construed as limitations on the scope of the claimed invention, but as exemplifications of the preferred embodiments thereof.

The following Examples illustrate several embodiments.

### 6. EXAMPLES

### 6.1 Preparation of Synthons Bearing Partial Metal Chelates

### Example 6.1.1

### Synthesis of N-α-FMOC—N-ε-di-tert-butylacetyl lysine

The following is a description of the synthesis of N-α-FMOC—N-ε-di-tert-butylacetyl lysine (IDA-lys, structure below), which can be subsequently used as a synthon to introduce an IDA moiety into a peptide, peptide nucleic acid probe, or any other probe that synthons can be coupled to via amide bond formation.

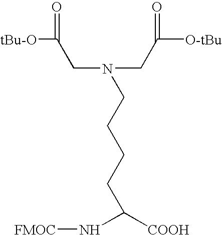

N-α-CBZ-lysine (Nova Biochem; 4.34 mmol, 1.218 g), bromo-tert-butylacetate (21.72 mmol, 3.203 mL), and K₂CO₃ (17.36 mmol, 2.402 g) were dissolved in 50 mL of 70% methanol/30% water. The reaction was stirred for 4 hours at 35° C. The solvent was evaporated under reduced pressure to yield a white residue. Water (10 mL) was added and the mixture centrifuged at 4000 RPM for 10 minutes, producing a pale yellow oil. The aqueous layer was removed, and an additional 5 mL water added to the oil, resulting in dissolution of the oil upon vortexing. This aqueous solution was extracted with hexane 3 times, followed by 3 extractions with ethyl acetate. The ethyl acetate extracts were combined and evaporated under a stream of nitrogen to yield a pale yellow oil, N-α-CBZ-N-ε-di-tert-butylacetyl lysine (1.31 g, 59%).

The CBZ protecting group of this product was removed by dissolving it in methanol (10 mL) and adding it to 135 mg of palladium on activated carbon (10% Pd) in a round bottom flask. The flask was evacuated and repressurized with H₂, and stirred under this H₂ atmosphere for 2 hours. The palladium-carbon was then filtered off, and the solvent evaporated to yield N-ε-di-tert-butylacetyl lysine (2.4 mmol, 897 mg, 93%).

This product was then FMOC protected at the α-amine by dissolving the derivatized amino acid in a THF solution (12 mL) containing FMOC—OSu (2.16 mmol, 728 mg). NaHCO₃ (4.8 mmol, 403 mg) was then added, and the mixture stirred as a suspension for 2 days. The mixture was then filtered, dried over Na₂SO₄, and the solvent then evaporated to an oily residue. The crude product was dissolved in minimal ethyl acetate and purified on a silica flash column. Hydrophobic impurities were eluted with 50:50 ethyl acetate:hexane, then the desired product was eluted with ethyl acetate+1% acetic acid. The fractions containing product were pooled and extracted with 5% NaHCO₃, then with saturated NaCl, and dried over Na₂SO₄. Evaporation of solvent yielded 680 mg of white solid (26% overall yield), >95% pure by LC-MS.

In an alternative synthesis, N-α-CBZ-lysine is substituted with N-α-FMOC-lysine (Nova Biochem) in the initial step. Following reaction as above with bromo-tert-butylacetate and K₂CO₃, the reaction mixture is worked up and the product purified by silica flash chromatography as above. However, deprotection of the α-FMOC group is a competing reaction in this protocol.

Using a similar procedure, the IDA group is introduced to other protected amino acids with primary amines, including but not limited to derivatives of ornithine, diaminobutyric acid, and diaminopropionic acid.

### Example 6.1.2

### Synthesis of N-α-FMOC-β-(N-α-di-tert-butylacetyl lysine-tert-butyl ester) aspartic acid

The following is a description of the synthesis of N-α-FMOC-β-(N-α-di-tert-butylacetyl lysine-tert-butyl ester) aspartic acid (NTA-asp, structure below) which is subsequently used to introduce an NTA moiety into a peptide, peptide nucleic acid probe, or any other probe that synthons can be coupled to via amide bond formation.

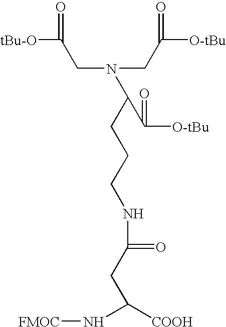

N-ε-CBZ lysine-tert-butyl ester (Advanced Chemtech; 2.88 mmol, 1.07 g), bromo-tert-butylacetate (17.3 mmol, 3.37 g), and K₂CO₃ (28.8 mmol, 3.98 g) were dissolved in 50 mL of acetonitrile and refluxed overnight. Following filtration of the K₂CO₃, the solvent was evaporated to a residual oil, 2.8 g. The oil was dissolved in 2430 diethyl ether (10 mL) and washed sequentially with 10% Na₂CO₃, water, and saturated NaCl, then dried over Na₂SO₄. The crude product was purified on a silica flash column eluted with 20% ethyl acetate 80% hexane, yielding N-ε-CBZ-N-α-di-tert-butylacetyl lysine tert-butyl ester as a colorless oil (1.5 g, 92%).

The N-ε-CBZ protecting group was removed by dissolving the oil in ethanol (30 mL) and slowly adding 150 mg of palladium on activated carbon (10% Pd). The vessel was evacuated and repressurized with H₂, and stirred under this H₂ atmosphere for 2 hours. The palladium-carbon was then filtered off, and the solvent evaporated to yield N-α-di-tert-butylacetyl lysine tert-butyl ester as a colorless oil (2.6 mmol, 1.1 g, 96%).

A portion of this oil (600 mg, 1.4 mmol) was dissolved in DMF (5 mL) and added to FMOC-aspartic anhydride (645 mg, 1.8 mmol, prepared as described previously by Yang and Su, J. Org. Chem., 51(26):5186, 1986), and stirred at room temperature for 1 hour. The DMF was evaporated under high vacuum and the crude product purified on a silica flash column eluted with ethyl acetate+1% acetic acid. The fractions containing product were washed sequentially with 5% NaHCO₃, water, 0.1N HCl, and saturated NaCl, then dried over Na₂SO₄. Evaporation of solvent yielded 318 mg of white solid (26% overall yield), >95% pure by LC-MS.

### Example 6.1.3

### Synthesis of a Synthon

### Bearing a Partial Metal Chelate and a Label

The following is a description of the synthesis of synthons bearing both a partial metal chelate and a label, and having the following general structure:

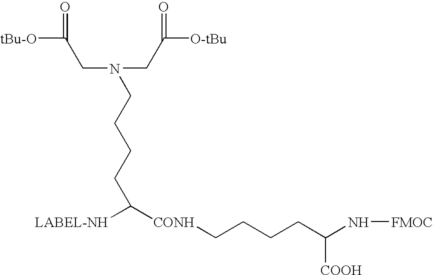

Such synthons can be used to introduce both an IDA and label moiety in a single coupling step into a peptide, peptide nucleic acid probe, or any other probe that synthons can be coupled to via amide bond formation. In this example, synthons were prepared wherein the label was either fluorescein or DABCYL, although using a similar procedure alternative synthons is prepared that bear the NTA group instead of the IDA group and/or that bear numerous other labels.

To prepare these synthons, N-ε-di-tert-butylacetyl lysine (an intermediate in the synthesis described in Example 6.1.1) was conjugated with the appropriate label (either fluorescein succinimide or DABCYL succinimide, both from Molecular Probes, Eugene Oreg.). These reactions were performed by solution phase synthesis in DMF (sufficient volume to dissolve reactants) using 1 equivalent of the reactive label derivative, 2.5 equivalents of N-ε-di-tert-butylacetyl lysine, and 5 equivalents of DIPEA. These solutions were allowed to react with stirring for 12 hours. The DMF solutions were then placed on a rotary evaporator and most of the DMF removed in vacuo at 35° C. For each reaction, the small amount of residual DMF was then diluted with ethyl acetate and applied to a silica flash column conditioned with hexane. Elution of the desired products was effected with a 50:50 mixture of ethyl acetate:methanol.

These products, N-α-LABEL-N-ε-di-tert-butylacetyl lysine (where LABEL=DABCYL or fluorescein) were then coupled to the ε-amine of N-α-FMOC lysine. This was achieved by pre-activating the carboxylate moieties of the labeled lysine derivatives with 1 equivalent of diisopropylcarbodiimide and 2 equivalents of N-hydroxysuccinimide in DMF to form the respective succinimide esters. N-α-FMOC lysine (2 equivalents) was then added to each reaction solution. After stirring for 12 hours, the DMF solutions were placed on a rotary evaporator and most of the DMF removed in vacuo at 35° C. For each reaction, the small amount of residual DMF was then diluted with ethyl acetate and applied to a silica flash column conditioned with hexane. Elution of the desired products was effected with ethyl acetate+1% acetic acid. The fraction containing the product was washed with aqueous sodium bicarbonate to remove the acetic acid, then with saturated sodium chloride. The ethyl acetate solution was dried over anhydrous sodium sulfate and rotovapped to a highly colored solid.

### Example 6.1.4

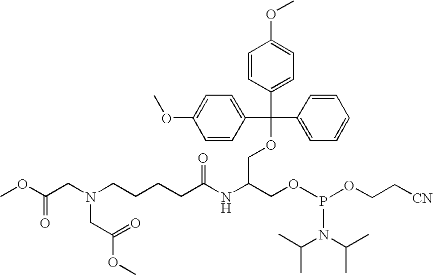

### Synthesis of [(4-{2-[Bis-(4-methoxy-phenyl)-phenyl-methoxy]-1-[(2-cyano-ethoxy)-diisopropylamino-phosphanyloxymethyl]-ethylcarbamoyl}-butyl-methoxycarbonylmethyl-amino]-acetic acid methyl ester

The following is a description of the synthesis of [(4-{2-{Bis-(4-methoxy-phenyl)-phenyl-methoxyl}-1-[(2-cyano-ethoxy-diisopropylamino-phosphanyloxymethyl]-ethylcarbamoyl}-butyl)-methoxycarbonylmethyl-amino]-acetic acid methyl ester which can be subsequently used to introduce partial metal chelators into a DNA or RNA molecule by standard synthesis methods.

5-tert-Butoxycarbonylamino-pentanoic acid (115 mmol, 25 g) was dissolved in 400 mL methanol and 20% Cs₂CO₃ (aq) (125 mL) was added dropwise via addition funnel until pH=7. The methanol was evaporated then the product was diluted with water and lypholized, then resuspended in DMF (250 mL). Benzyl bromide (104 mmol, 12 mL) was added to the solution and stirring was continued for 6 hours then the solid was filtered from the solution and the DMF was removed in vacuo. The product was dissolved in ethyl acetate and washed with 10% Na₂CO₃ (aq), water, brine, then dried over sodium sulfate and the solvent was evaporated yielding the 5-tert-butoxycarbonylamino-pentanoic acid benzyl ester (30.4 g, 86%).

The benzyl ester was dissolved in 100 mL DCM then 20 mL TFA was added and the reaction was stirred for 1 hour, then 20 mL TFA was added again and stirring continued for 2 hours then the solvent was removed in vacuo to give the deprotected amine as a clear oil. The product was used without further purification.

5-Amino-pentanoic acid benzyl ester (46 mmol, 9.5 g) and methyl bromoacetate (92 mmol, 14.1 g) were dissolved in 70 mL anhydrous acetonitrile. Solid K₂CO₃ (177 mmol, 24.5 g) was added and the mixture was brought to reflux for 20 hours then the solid was removed by filtration and the solution was concentrated in vacuo. The product was purified by silica gel chromatography (3:1 hexanes:ethyl acetate to 100% ethyl acetate) to a clear oil, 5-(bis-methoxycarbonylmethyl-amino)-pentanoic acid benzyl ester (4.63 g, 29%).

The benzyl ester was cleaved by dissolving a portion of the oil (7.3 mmol, 2.56 g) in 40 mL ethanol, then 200 mg solid Pd/C was added. The reaction flask was charged with H₂ (g) and stirred under positive H₂ (g) pressure for 16 hours. The Pd/C was filtered from the solution, and the solvent was then evaporated to give 5-(bis-methoxycarbonylmethyl-amino)-pentanoic acid as a clear oil (1.84 g, 96%).

The carboxylic acid (7.04 mmol, 1.84 g) was combined with pentafluorophenol (7.71 mmol, 1.42 g) in 10 mL cold ethyl acetate. After 5 minutes a solution of dicyclohexylcarbodiimide (7.76 mmol, 1.60 g) in 10 ml cold ethyl acetate was added to the stirring reaction solution. After stirring for 50 min at 0° C., the mixture was allowed to settle for 16 hours at −4° C. The solid precipitate was filtered from the reaction solution and the solvent was removed in vacuo, then the product was resuspended in 10 mL cold ethyl acetate and the newly formed precipitate was filtered from the reaction solution. This cycle of filtration and resuspension was repeated until solid was absent from the product, 5-(bis-methoxycarbonylmethyl-amino)-pentanoic acid pentafluorophenyl ester, which appeared as a slightly yellow oil (2.91 g, 97%).

Serinol (6.6 mmol, 600 mg) was dissolved in 30 mL anhydrous DMF then DIPEA (19.8 mmol, 3.56 mL) was added and the solution was cooled stirring over an ice bath for min. A cold solution of the active pentafluorophenol ester (6.6 mmol, 2.83 g) in 30 mL anhydrous DMF was added to the serinol solution and reaction was warmed to 25° C. with stirring for 16 hours. The DMF was removed in vacuo and the product was purified by silica gel chromatography (9:1 chloroform:methanol) to give the product {[4-(2-hydroxy-1-hydroxymethyl-ethylcarbamoyl)-butyl]-methoxycarbonylmethyl-amino}-acetic acid methyl ester as a clear oil (1.81 g, 82%).

The diol (5.26 mmol, 1.76 g) was dissolved in 5 mL anhydrous pyridine and added to a solution of 4,4′-dimethoxytrityl chloride (5.25 mmol, 1.78 g) in 10 mL anhydrous pyridine then the orange solution was stirred for 2 days, then 3 mL of methanol was added to the reaction solution and stirring was continued for 10 minutes and the solvent was then removed in vacuo. The product oil was dissolved in 40 mL ethyl acetate and washed with 5% NaHCO₃ (3×40 mL), and brine (1×40 mL), then dried over sodium sulfate and concentrated in vacuo. The product was purified by silica gel chromatography (100% chloroform to 9:1 chloroform:methanol) to yield the dimethoxytrityl protected product [(4-{2-[bis-(4-methoxy-phenyl)-phenyl-methoxy]-1-hydroxymethyl-ethylcarbamoyl}-butyl)-methoxycarbonylmethyl-amino]-acetic acid methyl ester as a clear oil (1.69 g, 51%)

The mono DMT protected alcohol (310 μmol, 200 mg) was dissolved in freshly prepared anhydrous acetonitrile and added to a solution of tetrazole (310 μmol, 688 μL, 0.45 M) followed by the addition of 2-cyanoethyl N,N,N′-tetraisopropylphosphordiamidite (310 μmol, 93 mg). Th reaction was stirred for one hour then the solvent was removed in vacuo. The product was taken up in ethyl acetate (10 mL) and washed with 5% NaHCO₃ (aq) (3×10 mL), and brine (1×10 mL), then dried over sodium sulfate and concentrated in vacuo. The product was purified by silica gel chromatography (ethyl acetate) to yield the CE-phosphoramidite [(4-{2-[bis-(4-methoxy-phenyl)-phenyl-methoxy]-1-[(2-cyano-ethoxy)-diisopropylamino-phosphanyloxymethyl]-ethylcarbamoyl}-butyl)-methoxycarbonylmethyl-amino]-acetic acid methyl ester as a clear oil (50 mg, 19%).

### 6.2 Assembly of Probes Using Synthons

### Example 6.2.1

### Synthesis of a PNA Probe with Two IDA Moieties

Probe: lys(IDA)-TGT ACG TCA CAA CTA-lys(IDA) having the following molecular structure:

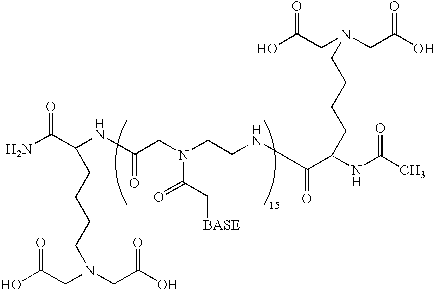

Note that the C-terminus is not a carboxylic acid, but rather a primary amide; this arises from the linker used in the solid-phase synthesis. Note also the N-terminus is acetylated. Both of these terminal groups reduce the likelihood of the termini contributing to any metal binding interaction, as may arise from carboxylic acids or primary amines.

Using the N-α-FMOC—N-ε-IDA lysine from Example 6.1.1 and commercially available FMOC-PNA monomers (Applied Biosystems, Foster City, Calif.), a PNA probe was prepared with an IDA chelating group at each terminus. The solid phase synthesis of this probe was performed using polystyrene synthesis resin with an XAL linker (Applied Biosystems). The nominal loading of the resin was 2 μmol. The FMOC protecting group was removed from the resin with 20% piperidine in DMF for 5 minutes. Then the resin washed with DMF to remove the piperidine. N-α-FMOC—N-ε-IDA lysine (10 μmol, 5 equivalents) was dissolved in 200 μL DMF. A solution of HATU (9 μmol, 4.5 equivalents) and HOBT (10 μmol, 5 equivalents) in 200 μL DMF was added to the N-α-FMOC—N-ε-IDA lysine solution, followed by DIPEA (20 μmol, 10 equivalents). This activated solution was then added to the resin, and agitated for 15 minutes. The resin was washed with DMF, and successful coupling confirmed by a negative Kaiser test. Any unreacted resin was capped with acetic anhydride (10% in DMF) for 3 minutes, and the resin again washed with DMF. The terminal FMOC protecting group was then removed with 20% piperidine in DMF for 5 minutes. Then the resin again washed with DMF.

Employing the same activation methods above (HATU+HOBT+DIPEA), PNA monomers were sequentially coupled to the probe to create the binding element having the sequence TGT ACG TCA CAA CTA. After completion of the binding element, a second N-α-FMOC—N-ε-IDA lysine was added following the same procedure as above. However, after the final FMOC group was removed, the probe was treated with the acetic anhydride solution to acetylate the N-terminus of the completed PNA probe. The resin was then washed sequentially with DMF and dichloromethane, and then dried in vacuo.

The completed PNA probe was cleaved from the resin and deprotected with a 90% TFA/10% m-cresol solution (100 μL). Complete deprotection of the tert-butyl ester protecting groups of the IDA moieties required 4 hours at room temperature. In this respect, synthesis of this probe differs from standard PNA probe synthesis, which typically requires a deprotection time of only a few minutes. At the end of the deprotection time, the probe was precipitated from the deprotection cocktail by the addition of diethyl ether (1.5 mL). The precipitated probe was pelleted by centrifugation in a microcentrifuge at 14,000 RPM for 10 minutes. The supernatant was then drawn off, and the white precipitate was washed with diethyl ether by vortexing. After washing, the precipitate was again pelletized by centrifugation. This washing procedure was repeated twice. The crude probe was then dried in vacuo.

The crude probe was resuspended in 400 μL of water+0.5% TFA and purified by reversed-phase HPLC using a Waters Delta-Pak C18 column, 7.8 mm×300 mm. The column was maintained at 50° C. in a column heater. The elution conditions were: solvent A, water+0.5% TFA; solvent B, acetonitrile+0.5% TFA; linear gradient of 5% solvent B to 35% solvent B over 30 minutes. The eluent was monitored by absorbance at 260 nm, and the desired probe product eluted after 20 minutes. The eluent containing the purified product was collected and lyophylized. The lyophylized powder was resuspended in 400 μL of water+0.5% TFA for analysis. The identity of the product was confirmed by electrospray ionization mass spectrometry using a Fisons VG Quattro mass spectrometer. The resultant electrospray mass spectrometry data were deconvoluted using the Maximum Entropy utility of the MassLynx software suite, confirming the mass of this species as 4575, equal to the calculated mass of the desired probe.

### Example 6.2.2

### Synthesis of a PNA Probe with Two IDA Moieties and Two Spacer Moieties

Probe: lys(IDA)-O-TGT ACG TCA CAA CTA-O-lys(IDA),
where the spacer moiety O═

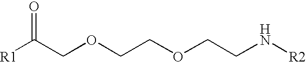

and R1 and R2 are adjacent monomers in the polymer chain. This spacer element is available commercially as the FMOC-protected derivative, FMOC-AEEG (Applied Biosystems).

This probe is comprised of the same binding element and possesses the same pair of IDA chelating groups as the probe in Example 6.2.1, but employs the short spacer element AEEG between the binding element and the chelators. It was prepared using the same solid phase synthesis techniques and reagents as in Example 6.2.1, with the addition of FMOC-AEEG. This synthon was added using the same ratio of activating agents (HATU, HOBT, DIPEA) as for the PNA monomers and for N-α-FMOC—N-ε-IDA lysine.

The purification of this probe was performed as described in Example 6.2.1. Addition of the two AEEG spacer moieties did not significantly affect the retention of the probe on the HPLC column. The purified probe yielded the correct mass of 4865 by electrospray mass spectrometry.

### Example 6.2.3

### Synthesis of a PNA Probe with an NTA Moiety and a Hexahistidyl Moiety

Probe:
asp(NTA)-TGT ACG TCA CAA CTA-his-his-his-his-his-
his

Using the N-α-FMOC-β-(N-α-di-tert-butylacetyl lysine-tert-butyl ester) aspartic acid from Example 6.1.2 and commercially available FMOC-PNA monomers (Applied Biosystems) and FMOC-histidine(trityl) (Nova Biochem), a PNA probe was prepared with an NTA chelating group at the C-terminus and a hexahistidyl sequence at the N-terminus. The solid phase synthesis of this probe was performed using the same solid-phase synthesis techniques as in Example 6.2.1. Following coupling of the final PNA monomer (A), six consecutive additions of FMOC-histidine(trityl) were carried out. The final histidine residue was N-acetylated with acetic anhydride, as in Example 6.2.1.

Deprotection of this probe in 90% TFA 10% m-cresol was found to be complete in about 3 hours. The trityl protecting groups of the histidyl residues are rapidly removed along with the BHOC protecting groups of the PNA monomers, but the complete removal of the tert-butyl esters of the NTA moiety is relatively slow.

Purification of this probe was carried out under the same HPLC conditions as described in Example 6.2.1.

### Example 6.2.4

### Synthesis of a PNA Probe with Two IDA Moieties and an Interactive Label Pair (DABCYL/EDANS)

Probe:
lys(DABCYL)-lys(IDA)-TGT ACG TCA CAA CTA-lys(IDA)-
glu(EDANS)

This probe is similar to that described in Example 6.2.1, but contains the additional elements of an interactive label pair in the form of the FRET pair EDANS and DABCYL. The synthons used to incorporate the elements of the label pair are commercially available (Nova Biochem): N-α-FMOC-β-EDANS glutamic acid and N-α-FMOC—N-ε-DABCYL lysine, which contain the respective labels conjugated to the side chains of the respective FMOC-protected amino acid derivatives. The other synthons needed to synthesize this probe are the N-α-FMOC—N-ε-IDA lysine from Example 6.1.1 and the commercially available PNA monomers (Applied Biosystems).

In this example, the label pair represents the terminal groups and the IDA moieties are positioned between the labels and the binding element. Subsequent examples will illustrate alternative arrangements.

Using the same solid-phase synthesis techniques as described in Example 6.2.1, the synthons described above were fully assembled on the synthesis resin. Lys(DABCYL) was first coupled to the resin, followed by lys(IDA), fifteen PNA monomers, another lys(IDA), and finally glu(EDANS). After the initial lys(DABCYL) coupling, the resin was an intense orange color, owing to the presence of the DABCYL chromophore. The DABCYL and EDANS moieties are stable to the reagents and conditions used in the synthesis, cleavage, and deprotection steps. The N-terminal amine of glu(EDANS) was acetylated as described for the probes above.

Following cleavage of the probe from the resin as in Example 6.2.1, a deprotection time of 4 hours allowed the removal of the tert-butyl groups from the IDA moieties. The probe was then purified by HPLC using the same column and solvents as in Example 6.2.1. However, the addition of the hydrophobic DABCYL and EDANS moieties resulted in greater retention of the probe on the C18 chromatographic resin, necessitating the use of a higher organic fraction to elute the product. Accordingly, a linear gradient of 20% B to 60% B over 20 minutes was performed, with the desired product eluting after 13 minutes as a red solution. Electrospray mass spectrometry confirmed the identity of this product with a mass of 5333.

### Example 6.2.5

### Synthesis of a PNA Probe with Two IDA Moieties and an Interactive Label Pair (DABCYL/TMR)

Probe:
lys(DABCYL)-lys(IDA)-TGT ACG TCA CAA CTA-lys(TMR)-
lys(IDA)

This probe is similar to that described in Example 6.2.4, but the label pair includes the fluorophore tetramethylrhodamine (TMR) in place of EDANS. Additionally, the geometry of this probe is somewhat different, with one of the labels (DABCYL) at the C-terminus, and one of the IDA chelating groups at the N-terminus. The reagents used were the same as in Example 6.2.4, but N-α-FMOC-β-EDANS glutamic acid was replaced with N-α-FMOC—N-ε-TMR lysine (Molecular Probes, Eugene Oreg.). The synthesis and purification of this probe was carried out in the same manner as given in Example 6.2.4.

### Example 6.2.6

### Synthesis of a PNA Probe with Two IDA Moieties and an Interactive Label Pair (DABCYL/Fluorescein)

Probe:
lys(IDA)-lys(DABCYL)-TGT ACG TCA CAA CTA-cys
(fluorescein)-lys(IDA)

This example illustrates a method of using a cysteine residue to introduce a reactive thiol to the probe, which can then be used as a site for the introduction of a label. The commercially available derivative FMOC-cys(t-butylthio) (Nova Biochem) was used to accomplish this. This probe also represents a different geometry from those in the previous two examples, with the two IDA chelating groups at opposite termini of the probe, and the two labels between an IDA moiety and the binding element.

The initial synthetic techniques in preparing this probe were identical to those given in Examples 6.2.1 through 6.2.5 to create the sequence shown above. Following the coupling of the fifteen PNA monomers, a cys(t-butylthio) residue was added by the same coupling chemistry, followed by a second lys(IDA). The N-terminal FMOC was removed and the amino terminus acetylated with acetic anhydride as in Examples 6.2.1 through 6.2.5. The resin was then extensively washed to remove residual acetic anhydride.

The resin was then suspended in a 50% solution of the disulfide reducing agent β-mercaptoethanol (BME, Calbiochem) in DMF, and agitated for 12 hours. This lengthy exposure to BME effectively removes the t-butylthio protecting group from the cysteine residue. Due to the pungent stench of BME, this step was performed in a laboratory fume hood. The resin was again washed extensively with DMF (in the fume hood) to remove any residual BME.

A fluorescein label was then conjugated to the deprotected thiol of the cysteine residue using the thiol-reactive derivative fluorescein-5-maleimide (Molecular Probes, Eugene Oreg.). This was done by dissolving a 5-fold excess of fluorescein-5-maleimide in 400 μL of DMF and adding this solution to the resin containing the deprotected cysteine residue. The reaction vessel was wrapped in foil to protect the solution from light (fluorescein-5-maleimide is light-sensitive) and agitated for 2 hours. The resin was then rinsed sequentially with DMF and DCM.

Cleavage, deprotection, and purification were conducted as in Example 6.2.4. The fluorescein moiety is stable to the deprotection conditions (90% TFA). The desired mass of 5487 was observed for the purified probe by mass spectrometry.

One skilled in the art of fluorescence labeling will recognize that a variety of fluorophores are commercially available to conjugate to cysteine residues. Although fluorescein was used in this specific example, a wide assortment of fluorophores are available as thiol-reactive derivatives. The reactive elements of these derivatives include maleimides, haloacetamides, and isothiocyanates. Indeed other, non-fluorophore labels may also be conjugated to a cysteine residue using the methods described in this example.

### Example 6.2.7

### Synthesis of a PNA Probe with Two IDA Moieties and an Interactive Label Pair (Pyrene/Pyrene)

Probe:
cys(pyrene)-lys(IDA)-O-TGT ACG TCA CAA CTA-O-lys
(IDA)-cys(pyrene)

This example describes the synthesis of a probe in which the interactive label pair is comprised of two copies of the same label, the fluorophore pyrene. This probe synthesis combines several of the features described in previous examples, including the use of AEEG spacer elements, a pair of lys(IDA) chelating groups, and the use of FMOC-cys(t-butylthio) as a synthon for the introduction of labels. In this probe, the metal chelators and pyrene labels are separated from the binding element by the AEEG spacers.

This probe was prepared using the same solid-phase techniques and reagents described in the above examples. Following the coupling of the final cys(t-butylthio) residue, the N-terminus was acetylated and the t-butylthio protecting groups removed with BME, as in Example 6.2.6. The two deprotected cysteine residues were then simultaneously labeled with pyrene using the thiol-reactive derivative N-(1-pyrene) maleimide (Molecular Probes, Eugene Oreg.). Ten equivalents of this reagent were dissolved in 400 μL of DMF and applied to the resin for 2 hours. This reaction was protected from light due to the light-sensitivity of this reagent. Following rinsing of the resin, cleavage, deprotection, and purification were conducted as in Example 6.2.4.

### Example 6.2.8

### Synthesis of a PNA Probe with Two IDA Moieties and an Interactive Label Pair (QSY-7/Oregon Green)

Probe:
lys(QSY-7)-lys(IDA)-TGT ACG TCA CAA CTA-lys(IDA)-
cys(Oregon Green)

This probe is similar to that described in Example 6.2.6, but utilizes a different quencher (QSY-7™, Molecular Probes, Eugene Oreg.) and a different fluorophore (Oregon Green™, Molecular Probes, Eugene Oreg.). The synthesis is essentially the same, with the substitution of N-α-FMOC—N-ε-QSY-7 lysine (Molecular Probes, Eugene Oreg.) for the N-α-FMOC—N-ε-DABCYL lysine, and the substitution of Oregon Green maleimide (Molecular Probes, Eugene Oreg.) for the fluorescein maleimide.

### Example 6.2.9

### Synthesis of a PNA Probe with Two IDA Moieties and an Interactive Label Pair (BHQ-1/Oregon Green)

Probe:
lys(BHQ-1)-lys(IDA)-TGT ACG TCA CAA CTA-lys(IDA)-
cys(fluorescein)

The probe of this example is similar to the probes described in Examples 6.2.6 and 6.2.8, however its synthesis is notably different. This arises from the fact that the alternative quencher Black Hole Quencher™-1 (Biosearch Technologies, Novato Calif.) is used, and it is not commercially available as an FMOC-protected synthon for peptide or peptide nucleic acid synthesis. However, it is available as an amine-reactive N-hydroxysuccinimide ester. By employing an amine-reactive quencher derivative (BHQ-1 succinimide ester, Biosearch Technologies, Novato Calif.) and a thiol-reactive fluorophore derivative (fluorescein maleimide, Molecular Probes, Eugene Oreg.), the probe was synthesized in the following manner.

Following the same solid-phase synthesis protocols described in the above examples, the first residue attached to the synthesis resin was N-α-FMOC—N-ε-ALOC lysine (Nova Biochem). The ALOC protecting group on the lysine side chain is an orthogonal protecting group, meaning that it is stable to all of the synthesis steps used in standard FMOC coupling chemistry, but can be removed independently. Following the removal of the FMOC group from this residue, the remainder of the probe was synthesized in the usual fashion according to the sequence shown above. After N-terminal acetylation, the cys (t-butylthio) residue was deprotected with BME as described in Example 6.2.6, and fluorescein maleimide conjugated to the deprotected thiol as before.

Following this step, the ALOC protecting group of the C-terminal lysine residue was removed under an argon atmosphere by placing tetrakis(triphenylphosphine)palladium(0) (5 equivalents, 10 μmol, 11.5 mg, Aldrich, Milwaukee Wis.) along with the dry resin in a sealable, fritted reaction column. In a separate vial, 150 μL of chloroform were combined with 4 μL of glacial acetic acid and 2 μL of N-methylmorpholine. An airtight syringe was flushed with argon, then used to transfer the chloroform solution to the reaction column. The column was then sealed and agitated for 2 hours. Following this reaction time, deprotection of the amine was confirmed by a positive Kaiser test. The resin was then sequentially washed with: DMF (3×); 0.5% DIPEA in DMF (3×); 0.5% sodium diethyldithiocarbamate in DMF (3×); DMF (3×); DCM (3×).

The now-deprotected ε-amino group of the C-terminal lysine was then conjugated with BHQ-1 succinimide ester by dissolving 5 equivalents of this reagent in DMF and adding it to the resin. This reaction was allowed to agitate for 12 hours. This long reaction time was necessary to achieve complete conjugation, probably due to steric constraints associated with labeling the ε-amine of the C-terminal residue after building an extensive backbone (16 residues) from its α-amine. The resin was then washed again with DMF and DCM. Following this conjugation, the probe was fully assembled. It was then cleaved, deprotected, and purified as described in Example 6.2.4.

### Example 6.2.10

### Synthesis of a PNA Probe with Two IDA Moieties and an Interactive Label Pair (DABCYL/Fluorescein)

Probe: lys(lys(α-DABCYL-ε-IDA)-TGT ACG TCA CAA CTA-lys(α-fluorescein-ε-IDA) having the following molecular structure:

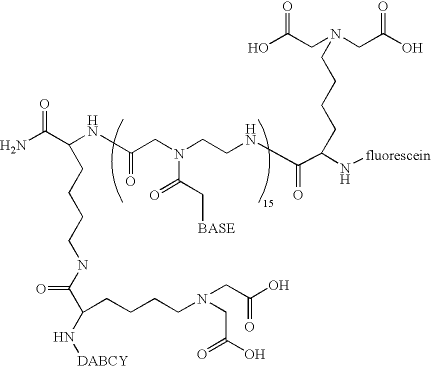

In all of the above examples of synthesis of probes containing an interactive label pair, the probes were prepared using separate synthons containing the metal chelating groups and the label moieties. These synthons served as ‘building blocks’ which were assembled in the desired order during solid-phase synthesis. Consequently, in each of these probes, the metal chelating moieties were separated from the label moieties by numerous covalent bonds, including two amino acid side chains and at least one peptide bond of the probe backbone. In some applications, it may be desirable for the labels to be located closer to the metal chelation sites in order, for example, to maximize the interaction between the labels when the probe is in the closed conformation. To achieve this goal, this example illustrates a method by which a label and a chelator was incorporated into a single lysine residue, using both of the lysine amino groups as reactive sites. We have also applied this method to other amino acids with amino side chains, including derivatives of ornithine (orn), diaminobutyric acid (DAB), and diaminopropionic acid (DAP) using methods described in Example 6.1.1. In this manner, the spatial separation between the chelating group and the label was varied according to the number of bonds between the amino groups of the diamino acid.

As in Example 6.2.9, the solid-phase synthesis of this probe began with the coupling of N-α-FMOC—N-ε-ALOC lysine (Nova Biochem) to the synthesis resin. This was followed by the standard sequential addition of 15 PNA monomers, then an N-terminal N-α-FMOC—N-ε-IDA lysine. Following the removal of the N-terminal FMOC group, the N-terminus was not acetylated. Instead, a 10-fold excess of the amine-reactive fluorescein derivative fluorescein succinimide ester (Molecular Probes, Eugene Oreg.) in DMF was added to the resin, along with an equimolar quantity of DIPEA, and agitated for 12 hours. In this fashion, fluorescein was conjugated to the α-amine of the N-terminal lysine residue, the same lysine residue which bears the IDA group on its ε-amine.

Following this step, the ALOC protecting group of the C-terminal lysine residue was removed using the same procedure described in Example 6.2.9. Once the ε-amine of this residue was deprotected, it was coupled with N-α-FMOC—N-ε-IDA lysine. As noted in Example 6.2.9, this reaction at the ε-amine of the C-terminal lysine residue required a longer reaction time (12 hours) to reach completion, presumably due to steric effects. When this reaction was complete, the FMOC group was removed and the α-amine of the lysine (IDA) residue was conjugated to DABCYL using the amine-reactive derivative DABCYL succinimide ester (Molecular Probes, Eugene Oreg.). This reagent (10-fold excess) was dissolved in DMF and allowed to react for 12 hours. With the completion of this step, probe assembly was complete. Cleavage, deprotection, and purification were carried out as in Example 6.2.4.

### 6.3 Physical Properties of Probes

### Example 6.3.1

### Specificity Determination by Absorbance Spectroscopy (Melting Curve Analysis)

For oligonucleotide probes which lack a label, the binding of the probe to a target nucleotide sequence is monitored by absorbance spectroscopy. This technique exploits the fact that the molar absorptivity of the nucleobases is lower in the duplex (bound) state, and thus the absorbance of a solution containing probe and target decreases upon target binding. By slowly varying the temperature of the solution, the ratio of bound to unbound probe is altered. Monitoring the absorbance during a temperature ramp over a range from fully bound to fully unbound probe produces a sigmoidal binding curve, the center of which is termed the melting temperature (Tₘ). The melting temperature represents the temperature at which half of the probe is bound to target, and is a concentration-dependent parameter. To determine the Tₘ from the sigmoidal melting curve, we employed the first derivative method, in which the slope of the curve (the first derivative) is determined at each temperature along the transition from bound to unbound probe. The temperature at which the slope (and thus the first derivative) is greatest is taken as the Tₘ; this is also the inflection point of the sigmoidal curve.

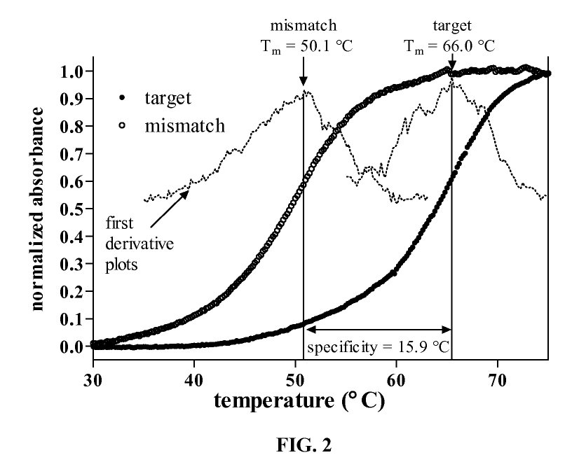

By comparing the Tₘ values of the probe-target complex and a complex between the probe and a mismatched target, the specificity of the probe can be quantified. The greater is the separation between these Tₘ values, the more specifically can the probe be used to detect the desired target. To illustrate this analysis, FIG. 2 shows the sigmoidal melting curves of the probe prepared in Example 6.2.2 with its DNA target (5′ ACA TGC AGT GTT GAT 3′) and with a DNA strand containing a single base mismatch in the middle of the sequence (5′ ACA TGC ATT GTT GAT 3′). Overlaid on these melting curves are the first derivative plots, the maxima of which represent the Tₘ values for the respective complexes. In this example, the Tₘ of the probe with its target was 66.0° C., 2980 while that with the mismatched sequence was 50.1° C. The specificity of the probe was quantified as the difference between these two values, 15.9° C. These curves were obtained in the absence of any transition metal, so the probe is not constrained by the intramolecular coordination complex (i.e., the ‘closed’ conformation does not exist). In a parallel experiment with added Ni⁺², a similar analysis yielded Tₘ values of 58.3° C. and 37.9° C. for the target and mismatch, respectively (not shown), for a specificity of 20.4° C. Thus, the introduction of the ‘closed’ conformation resulted in a specificity increase of 4.5° C. These data, along with those determined for other probes we have prepared, are tabulated in TABLE 3 in Example 6.3.5 below.

The data in this example were acquired using a Cary 3 spectrophotometer equipped with a 6×6 cell changer and Peltier temperature controller. The absorbance of the samples at 260 nm was recorded as the temperature was varied from 80° C. to 20° C. at a rate of 0.2° C. per minute. The samples themselves contained 500 nM probe and 500 nM target or mismatched DNA in 10 mM potassium phosphate buffer, pH 7.2, with 10 mM NaCl. The data acquisition and analysis was performed with the Cary Thermal software package.

### Example 6.3.2

### Effect of Metal Concentration on Tₘ

It is expected that when transition metal ions are introduced to a probe it will adopt a closed conformation and, the Tₘ value of the bound probe will decrease. This is due to the fact that the bound conformation of the probe must ‘compete’ with the closed conformation. The increase in specificity of constrained probes arose from the observation that the Tₘ of a probe-mismatch complex decreases more than the Tₘ of the probe-target complex. Thus, for a given probe, it is desirable to find conditions under which the Tₘ of the probe-mismatch complex is lowest.

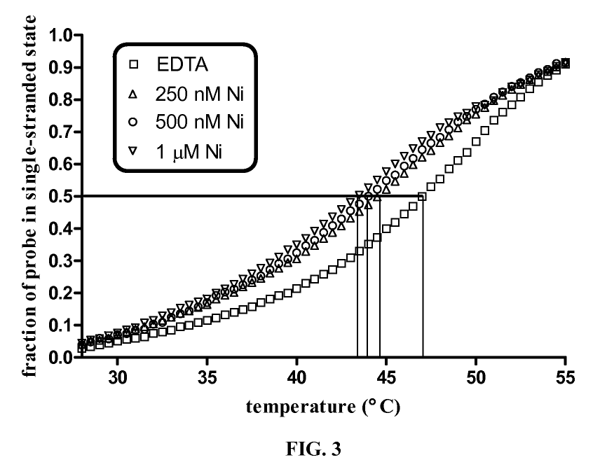

One such condition is the concentration of the transition metal ion. Using the method described in the previous example, the effect of different concentrations of transition metal ions upon the Tₘ values of probe-mismatch complexes were determined. In this example, data for the PNA probe prepared in Example 6.2.3, containing an NTA group at the C-terminus and a hexahistidine sequence at the N-terminus, along with data for a similar probe containing only 2 histidine residues at the N-terminus. FIG. 3 illustrates how melting curves for this dihistidine probe vary as a function of nickel concentration. Concentrations of probe and the single-base mismatched DNA sequence (as in Example 6.3.7) were 500 nM in this experiment. The vertical lines in the figure correspond to the Tₘs, which decreased with nickel concentration up to 1.0 μM; further increases in nickel concentration exerted a negligible effect on Tₘ.

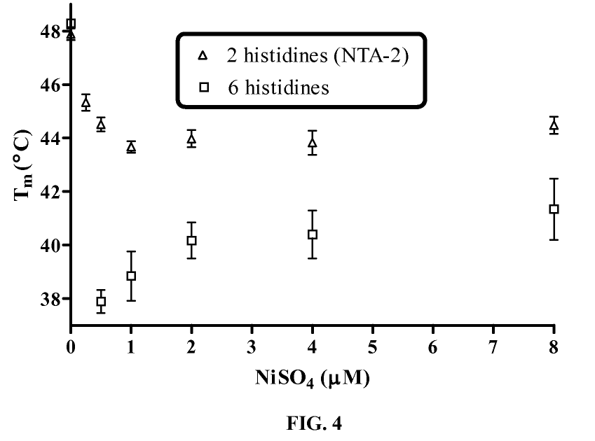

A similar experiment with the hexahistidine probe revealed a more complex behavior, with the Tₘ reaching its lowest point at 500 nM nickel, then slowly increasing with greater nickel concentrations. The contrasting behavior of these two probes is illustrated in FIG. 4, which plots Tₘ versus nickel concentration for both the dihistidine and hexahistidine probes. While the Tₘ values of the dihistidine probe (open triangles) exhibited a simple hyperbolic decrease, the values for the hexahistidine probe reached a minimum when the nickel concentration is equimolar with the probe (500 nM), then slowly increased. Without wishing to be bound by theory, we believe that this difference in behavior is due to the greater binding affinity of the hexahistidine moiety relative to the dihistidine moiety, which allowed it to bind nickel independently of the NTA group when excess nickel was present. In other words, in the presence of excess nickel, the hexahistidine group and the NTA group may bind separate metal ions, which precluded the formation of the closed probe conformation. Probes in this state behaved similarly as probes which have no metal ion bound at all, and thus the observed Tₘ increases.

### Example 6.3.3

### Effect of Different Transition Metals on Tₘ

Another condition expected to effect the Tₘ of probes was the identity of the transition metal used in a detection method. For instance, the Tₘ of the PNA probe described in Example 6.2.1 with a mismatched DNA sequence was determined in the presence of a variety of transition metal ions at various concentrations. The resulting data, determined as described in Examples 6.3.1 and 6.3.2, are tabulated in TABLE 2 below. The concentration of PNA probe and mismatched DNA target in these experiments was 500 nM.

TABLE 2
Effect of different transition metals on Tₘ

| Metal ion | ΔTₘ(max) (° C.)¹ | [M] at ΔTₘ(max) (μM)² |
|---|---|---|
| Ni⁺² | 12 | 2 |
| Cu⁺² | 6 | 1 |
| Co⁺² | 6 | 2-8 |
| Zn⁺² | 4 | 8 |
| Mn⁺² | <2 | — |
| Fe⁺² | <2 | — |
| Fe⁺³ | <2 | — |

¹The maximal observed decrease in Tₘ over a range of metal ion concentrations.
²The metal concentration at which the maximal decrease in Tₘ is observed.

As indicated in the table, the maximal effect on Tₘ was seen with Ni⁺², which decreased the melting temperature of the probe-mismatch complex by 12° C. at a concentration of 2 μM (a 4-fold excess relative to the probe). Ions of copper, cobalt and zinc yielded more modest Tₘ depression values, while ions of manganese and iron exhibited negligible effect at any concentration.

### Example 6.3.4

### Specificity Determination by Fluorescence Spectroscopy (Melting Curve Analysis)

For probes which contain a fluorophore/quencher interactive label pair, the binding of the probe to a target was monitored by observing changes in fluorescence. As with the use of absorbance spectroscopy described above, monitoring the fluorescence of a sample containing probe and target during a temperature ramp over a range from fully bound to fully unbound probe produced a sigmoidal binding curve. In this case, the fluorescence was observed to increase upon binding of the probe to its target. As with the absorbance melting curves, the Tₘ from a fluorescence curve was determined by the first derivative method (see Example 6.3.1). Again by comparing the Tₘ values of the probe-target complex and a complex between the probe and a mismatched target, the specificity of the probe is quantified.

For example, this analysis was performed with a probe labeled with fluorescein and DABCYL prepared as described in Example 6.2.10, except the synthons used were derived from ornithine (IDA) rather than lysine (IDA):

lys(orn(α-DABCYL-ε-IDA)-TGT ACG TCA CAA CTA-
orn(α-fluorescein-ε-IDA)

As described in Example 6.3.7, melting curves (and hence Tₘ values) were determined for this probe bound to both its perfect DNA target (5′ ACA TGC AGT GTT GAT 3′) and to a DNA target containing a single base mismatch (5′ ACA TGC ATT GTT GAT 3′). The difference between these Tₘ values was a quantitative measure of the specificity of the probe.

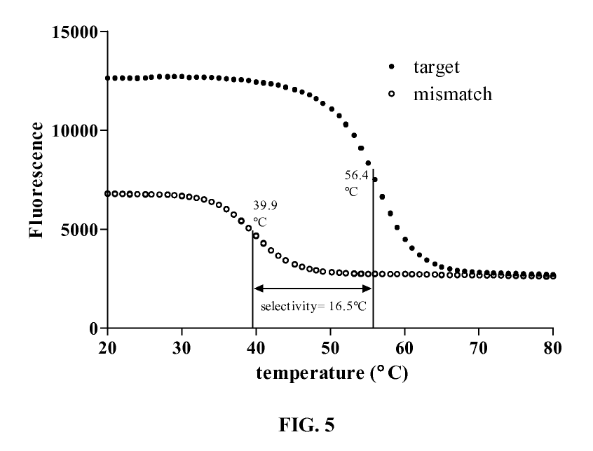

FIG. 5 illustrates these two melting curves of the probe in the presence of Ni⁺² (the first derivative curves are omitted from this example). At low temperatures, the probe was in the ‘open’ conformation and the fluorophore and quencher were spatially separated, resulting in relatively high fluorescence. Note that the fluorescence of the probe when bound to its correct target was in fact higher than when bound to the mismatched target. As the temperature was raised, the complex dissociates and the probe adopted its ‘closed’ conformation, placing the fluorophore and quencher in close proximity, therefore lowering the observed fluorescence. Note that the fluorescence of the two samples was essentially the same at higher temperatures, since in this state the conformation of the probe was independent of any targets which may been in the sample. The vertical lines again indicate the Tₘ values, which in this example, yielded a specificity of 16.5° C. These data, along with those determined for other probes we have prepared, are tabulated in TABLE 3 in Example 6.3.5 below.

The data in this example were acquired using an ABI Prism 7700 real-time PCR instrument. The fluorescence of the samples was recorded as the temperature was varied from 80° C. to 20° C. at a rate of 0.2° C. per minute. The samples themselves contained 100 nM probe and 100 nM target or mismatched DNA plus 100 nM NiSO₄ in 10 mM potassium phosphate buffer, pH 7.2, with 10 mM NaCl. The total sample volume was 100 μL. The data acquisition was performed with the ABI Prism software package. The data was analyzed by importing the resulting data files into GraphPad Prism 4.0. For each sample, the fluorescence at 520 nm (the emission maximum) was recorded at each temperature point. The background fluorescence (determined in a separate experiment with samples containing only buffer) was subtracted from these values, and the resulting data was plotted as shown in FIG. 5. A first derivative curve was then calculated for each melting curve and the Tₘ determined as in Example 6.3.1.

### Example 6.3.5

### Probe Specificities as a Function of Ni⁺²

Using the spectroscopic methods described in Examples 6.3.1 and 6.3.4 (for unlabelled and fluorescently labeled probes, respectively), Tₘ and specificity factor values have been determined for the set of PNA probes shown below in TABLE 3. The nomenclature used in this table to describe the probe composition is a C-terminal to N-terminal sequence with the monomeric units separated by hyphens, using the abbreviations provided in the table footnote. Accordingly, the abbreviated sequence “I-G-C(OG)-P₁₅-D-G-I” in TABLE 3 represents the probe, “lys(IDA)-gly-cys(Oregon Green)-TGT ACG TCA CAA CTA-lys(DABCYL)-gly-lys(IDA)”. Similarly, the abbreviated sequence “N—P₁₅—H₄” in TABLE 3 represents the probe “asp(NTA)-TGT ACG TCA CAA CTA-his-his-his-his”.

TABLE 3
Probe Specificities as a Function of Metal (Ni⁺²)

| | absence of metal | | | | | | |
|---|---|---|---|---|---|---|---|
| | (EDTA) | | | presence of metal (Ni⁺²) | | | Change In |
| | Target | Mismatch | Specificity Factor | Target | Mismatch | Specificity Factor | Specificity Factor |
| Probe | (T_{m-M}) | (T_{m-M}) | (ΔT_{m-M}) | (T_{m+M}) | (T_{m+M}) | (ΔT_{m+M}) | (ΔΔTₘ) |
| P₁₅ ^{1, 3} | 65.4 ⁴ | 49.5 | 15.9 | 65.0 | 49.9 | 15.1 | −0.8 |
| N-P₁₅ ^{1, 3} | 64.3 | 47.8 | 16.5 | 63.8 | 47.7 | 16.1 | −0.4 |
| H₂-P₁₅ ^{1, 3} | 66.6 | 50.3 | 16.3 | 66.0 | 50.4 | 15.6 | −0.7 |
| N-P₁₅-H ¹ | | 47.8 | | | 45.6 | | n.d. |
| N-P₁₅-H₂ ¹ | 63.5 | 47.8 | 15.7 | 61.7 | 43.8 | 17.9 | +2.2 |
| N-P₁₅-H₃ ¹ | 64.9 | 48.4 | 16.5 | 62.6 | 41.6 | 21.0 | +4.5 |
| N-P₁₅-H₄ ¹ | 65.4 | 50.1 | 15.3 | 61.0 | 41.5 | 19.5 | +4.2 |
| N-P₁₅-H₆ ¹ | 65.4 | 48.3 | 17.1 | 59.6 | 37.9 | 21.7 | +4.6 |
| H₂-P₁₅-N ¹ | 65.3 | 49.7 | 15.6 | 62.2 | 44.3 | 17.9 | +2.3 |
| I-P₁₅-H₂ ¹ | | 48.9 | | | 46.2 | | n.d. |
| I-P₁₅-I ¹ | 64.7 | 47.9 | 16.8 | 55.7 | 34.8 | 20.9 | +4.1 |
| I-O-P₁₅-O-I ¹ | 66.0 | 50.1 | 15.9 | 58.3 | 37.9 | 20.4 | +4.5 |
| N-P₁₅-N ¹ | 64.4 | 48.4 | 16.0 | 63.8 | 47.8 | 16.0 | 0.0 |
| I-G-C(OG)-P₁₅-D-G-I ² | 60.4 | | | 58.0 | | | n.d. |
| I1D-P₁₅-I1F ² | 60.5 | 46.6 | 13.9 | 56.2 | 39.0 | 16.2 | +2.3 |
| I1D-P₁₅-I2F ² | 57.2 | | | 55.2 | | | n.d. |
| I1D-P₁₅-I3F ² | 58.9 | | | 56.6 | | | n.d. |
| I1D-P₁₅-I4F ² | 59.3 | | | 57.0 | | | n.d. |
| I2D-P₁₅-I1F ² | 56.4 | | | 54.1 | | | n.d. |
| I2D-P₁₅-I2F ² | 62.6 | | | 50.4 | 34.4 | 16.0 | n.d. |
| I2D-P₁₅-I3F ² | 54.0 | | | 52.1 | | | n.d. |
| I2D-P₁₅-I4F ² | 57.1 | | | 53.3 | | | n.d. |
| I3D-P₁₅-I1F ² | 61.1 | | | 56.1 | | | n.d. |
| I3D-P₁₅-I2F ² | 55.3 | | | 53.6 | | | n.d. |
| I3D-P₁₅-I3F ² | 62.3 | 48.4 | 13.9 | 56.4 | 39.9 | 16.5 | +2.6 |
| I3D-P₁₅-I4F ² | 58.4 | | | 56.6 | | | n.d. |
| I4D-P₁₅-I1F ² | 60.4 | | | 57.6 | | | n.d. |
| I4D-P₁₅-I2F ² | 58.2 | | | 55.2 | | | n.d. |
| I4D-P₁₅-I3F ² | 59.8 | | | 56.8 | | | n.d. |
| I4D-P₁₅-I4F ² | | | | 58.7 | | | n.d. |

Abbreviations: P₁₅ = PNA sequence TGT ACG TCA CAA CTA; N = aspartate(NTA) (Examples 6.1.2 and 6.2.3)
I = lysine(IDA) (Examples 6.1.1 and 6.2.1); H = histidine (Example 6.2.3); O = AEEG spacer (Example 6.2.2); G = glycine; D = lysine(DABCYL) (Example 6.2.4); C(OG) = cysteine(Oregon Green) (Example 6.2.8); I1D = lys(DAP(α-DABCYL-β-IDA) (Example 6.2.10); I2D = lys(DAB(α-DABCYL-γ-IDA) (Example 6.2.10); I3D = lys(orn(α-DABCYL-δ-IDA) (Example 6.2.10); I4D = lys(lys(α-DABCYL-ε-IDA) (Example 6.2.10); I1F = DAP(α-fluorescein-β-IDA) (N-terminal residue only, Example 6.2.10); I2F = DAB(α-fluorescein-γ-IDA) (N-terminal residue only, Example 6.2.10); I3F = orn(α-fluorescein-δ-IDA) (N-terminal residue only, Example 6.2.10); I4F = lys(α-fluorescein-ε-IDA) (N-terminal residue only, Example 6.2.10).
¹ Determined by absorbance spectroscopy (see Example 6.3.1). Probe concentration, 500 nM
² Determined by fluorescence spectroscopy (see Example 6.3.4). Probe concentration, 100 nM
³ Not probes of the subject invention (control probes).
⁴ All numerical values in units of Celsius.

As can be seen in the right-most column of data, probes typically displayed an increase in selectivity of 2-4° C. when nickel was included in the method (enabling the ‘closed’ conformation of the probe). The control probes with only a single partial metal chelation site (top three rows) exhibited a slight (<1° C.) decrease in selectivity in the presence of nickel. A probe containing two NTA groups (N—P₁₅—N) did not exhibit any change upon addition of nickel. Again, not wishing to be bound by theory, this can be understood given that the two tetravalent NTA groups collectively exceeded the ligand binding capacity of the Ni⁺² ion, so that only one NTA group can bind to a given metal ion at one time. Effectively, this probe behaved as though it lacked a second partial chelation site since the second site was unable to ‘share’ a single nickel ion. These results illustrate the importance of designing probes with appropriate partial chelation sites, as discussed elsewhere in the specification.

### Example 6.3.6

### Determination of the Conformational Dependence of Fluorescence in Fluorophore/Quencher Labeled Probes

With interactively labeled probes, one functional consideration in their use is how great is the change in label signal upon binding of the probe to its target. As illustrated in FIG. 5 (Example 6.3.4), a probe with a fluorophore/quencher pair was expected to exhibit greater fluorescence when bound to target (open conformation) than when the coordination complex was intact (closed conformation). Moreover, it is expected that in the absence of metal, the fluorophore and quencher will not be held in close proximity, and thus the fluorescence will also be higher than when metal was present to hold the chelation sites together.

To assess these effects, a solution containing the fluorescently labeled probe was split into three aliquots. To the first is added a sufficient volume of a nickel stock solution to achieve a twofold excess of Ni⁺². To the second was added the one-half the volume of a second nickel stock solution of twice the nickel concentration, and one-half the volume of a target stock solution to again achieve a twofold excess of target (and the nickel concentration is the same as in the first sample). To the third was added a volume of buffer equivalent to the volume added to the other two samples. Thus, the three solutions contained the same concentration of probe, but one contained a twofold excess of nickel (thus the probe is in the closed conformation), one contained a twofold excess of nickel and target (probe in the open conformation), and the third contained neither nickel nor target (probe in an indefinite conformation). The fluorescence of these three samples was then determined using excitation and emission wavelengths appropriate for the fluorophore within the probe.

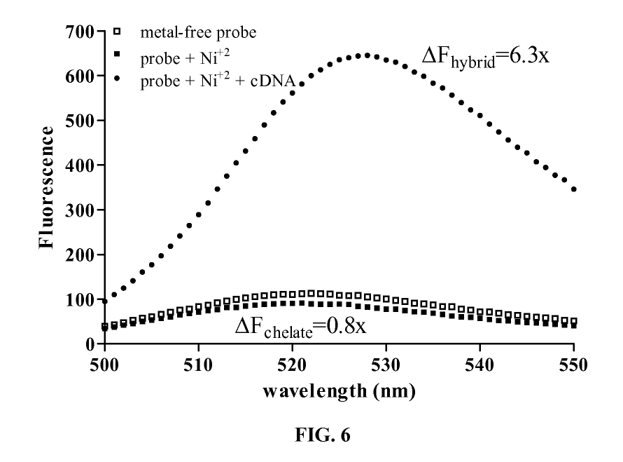

The data (fluorescence spectra) from such an experiment for the probe I-D-O—P₁₅—O—C(OG)-I are shown in FIG. 6. Although the absolute values of fluorescence were not physically meaningful (arbitrary units), the change in fluorescence from one state to another were the experimentally important parameters. These parameters are given in the ΔF values reported in the figure, which are factors by which the fluorescence changes from one state to another. In this case, the fluorescence of the nickel-bound closed conformation (ΔF_{chelate}) was 0.8× relative to the fluorescence of the indefinite conformation in the absence of nickel. In other words, formation of the closed conformation by the addition of nickel (and formation of the complete coordination complex) caused the fluorescence to decrease by a factor of 0.8, or 20%. Addition of target to this sample then caused an increase in fluorescence by a factor of 6.3× (ΔF_{hybrid}). This increase in fluorescence upon target binding represented the analytically useful signal in methods employing such probes.

These parameters, along with those determined for other probes we have prepared, are tabulated in TABLE 4 in Example 6.3.7 below.

The fluorescence spectra presented in this example were determined using an SLM 8100 fluorometer. The excitation wavelength was 488 nm. The samples contained 100 nM probe and appropriate concentrations of nickel and/or target DNA as described above. The buffer used was 10 mM potassium phosphate buffer, pH 7.2, with 10 mM NaCl.

### Example 6.3.7

### Fluorescence as a Function of Probe-Conformation

As described in Example 6.3.6 above, the fluorescence of labeled probes was determined in the absence of nickel (indefinite conformation), presence of nickel (closed conformation), and presence of target (open conformation). Changes in fluorescence upon formation of the coordination complex (ΔF_{chelate}) and upon target binding (ΔF_{hybrid}) were then calculated. The results for all probes tested in this fashion are tabulated in TABLE 4 below. As can be seen in the last column, the greatest values for ΔF_{hybrid} were generally seen with probes containing the FRET pair EDANS and DABCYL.

TABLE 4
Fluorescence as a Function of Probe-Conformation

| Probe composition | Ex/Em (nm) | −Ni⁺² | +Ni⁺² | +target | ΔF_{chelate} ¹ | ΔF_{hybrid} ² |
|---|---|---|---|---|---|---|
| D-P₁₅-E | 340/495 | 69 | 62 | 1120 | 0.9 | 18.1 |
| D-N-P₁₅-E-H₂ | 340/495 | 49 | 24 | 726 | 0.5 | 30.3 |
| D-N-P₁₅-H₂-E | 340/495 | 154 | 134 | 495 | 0.9 | 3.7 |
| N-D-P₁₅-H₂-E | 340/495 | 117 | 76 | 569 | 0.6 | 7.5 |
| N-D-P₁₅-E-H | 340/495 | 150 | 103 | 618 | 0.7 | 6.0 |
| N-D-P₁₅-E-H₂ | 340/495 | 110 | 86 | 722 | 0.8 | 8.4 |
| N-D-P₁₅-E-H₃ | 340/495 | 28 | 43 | 172 | 1.5 | 4.0 |
| N-D-P₁₅-E-H₄ | 340/495 | 18 | 9.4 | 68 | 0.5 | 7.2 |
| N-D-P₁₅-E-H₅ | 340/495 | 30 | 25 | 24 | 0.8 | 0.9 |
| N-D-P₁₅-E-H₆ | 340/495 | 39 | 0.8 | 9.1 | 0.01 | 11.7 |
| D-I-P₁₅-E-I | 340/495 | 13 | 10 | 57 | 0.8 | 5.7 |
| D-I-P₁₅-I-E | 340/495 | 17 | 14 | 59 | 0.8 | 4.3 |
| I-D-P₁₅-E-I | 340/495 | 6.5 | 4.0 | 50 | 0.6 | 12.5 |
| I-D-P₁₅-I-E | 340/495 | 17 | 14 | 45 | 0.8 | 3.3 |
| D-I-P₁₅-C(F)-I | 488/520 | 136 | 134 | 169 | 1.0 | 1.3 |
| D-I-P₁₅-I-C(F) | 488/520 | 846 | 853 | 838 | 1.0 | 1.0 |
| I-D-P₁₅-C(F)-I | 488/520 | 179 | 176 | 240 | 1.0 | 1.4 |
| I-D-P₁₅-I-C(F) | 488/520 | 124 | 121 | 165 | 1.0 | 1.4 |
| C(F)-I-P₁₅-I-D | 488/520 | 258 | 208 | 233 | 0.8 | 1.1 |
| I-C(F)-P₁₅-I-D | 488/520 | 215 | 164 | 239 | 0.8 | 1.5 |
| I-C(F)-P₁₅-D-I | 488/520 | 221 | 173 | 226 | 0.8 | 1.3 |
| I-D-P₁₅-C(OG)-I | 488/525 | 46 | 44 | 168 | 1.0 | 3.8 |
| D-I-P₁₅-I-C(OG) | 488/525 | 47 | 46 | 144 | 1.0 | 3.2 |
| I-D-P₁₅-C(AF)-I | 488/521 | 45 | 41 | 171 | 0.9 | 4.2 |
| D-I-P₁₅-I-C(AF) | 488/521 | 32 | 32 | 72 | 1.0 | 2.3 |
| D-O-I-P₁₅-I-O-C(F) | 488/520 | 122 | 110 | 397 | 0.9 | 3.6 |
| D-O-I-O-P₁₅-O-I-O-C(F) | 488/520 | 177 | 144 | 385 | 0.8 | 2.7 |
| D-O-I-P₁₅-I-O-C(OG) | 488/525 | 137 | 128 | 492 | 0.9 | 3.8 |
| D-O-I-O-P₁₅-O-I-O-C(OG) | 488/525 | 187 | 150 | 446 | 0.8 | 3.0 |
| D-I-O-P₁₅-O-I-C(OG) | 488/525 | 123 | 114 | 514 | 0.9 | 4.5 |
| D-I-O-P₁₅-O-I-O-C(OG) | 488/525 | 153 | 129 | 431 | 0.8 | 3.3 |
| I-D-O-P₁₅-O-C(OG)-I | 488/525 | 112 | 92 | 581 | 0.8 | 6.3 |
| I-D-O-P₁₅-O-C(OG)-O-I | 488/525 | 127 | 107 | 624 | 0.8 | 5.8 |
| I-G-C(OG)-P₁₅-D-G-I | 488/525 | 6325 | 5061 | 16445 | 0.8 | 3.2 |
| I-G-C(OG)-O-P₁₅-O-D-G-I | 488/525 | 9336 | 6980 | 16530 | 0.7 | 2.4 |
| C(OG)-G-I-P₁₅-I-G-D | 488/525 | 8236 | 7038 | 15410 | 0.9 | 2.2 |
| C(OG)-G-I-O-P₁₅-O-I-G-D | 488/525 | 9465 | 7098 | 16986 | 0.7 | 2.4 |
| I-G-C(OG)-P₁₅-Q-G-I | 488/525 | 745 | 1041 | 629 | 1.4 | 0.6 |
| I-G-C(OG)-O-P₁₅-O-Q-G-I | 488/525 | 9836 | 8305 | 11159 | 0.8 | 1.3 |
| C(OG)-G-I-P₁₅-I-G-Q | 488/525 | 1658 | 1599 | 3600 | 1.0 | 2.3 |
| C(OG)-G-I-O-P₁₅-O-I-G-Q | 488/525 | 3652 | 2603 | 4422 | 0.7 | 1.7 |
| I-O-C(OG)-P₁₅-D-O-I | 488/525 | 9012 | 6820 | 15518 | 0.8 | 2.3 |
| C(OG)-O-I-O-P₁₅-O-I-O-D | 488/525 | 16018 | 11780 | 19612 | 0.7 | 1.7 |
| I1D-P₁₅-I1F | 488/520 | 2715 | 2166 | 6071 | 0.8 | 2.8 |
| I1D-P₁₅-I2F | 488/520 | 846 | 753 | 1818 | 0.9 | 2.4 |
| I1D-P₁₅-I3F | 488/520 | 6514 | 6269 | 8107 | 1.0 | 1.3 |
| I1D-P₁₅-I4F | 488/520 | 1915 | 1502 | 5129 | 0.8 | 3.4 |
| I2D-P₁₅-I1F | 488/520 | 1242 | 1103 | 3226 | 0.9 | 2.9 |
| I2D-P₁₅-I2F | 488/520 | 1535 | 1303 | 5755 | 0.8 | 4.4 |
| I2D-P₁₅-I3F | 488/520 | 2243 | 2328 | 7025 | 1.0 | 3.0 |
| I2D-P₁₅-I4F | 488/520 | 1719 | 1631 | 7162 | 0.9 | 4.4 |
| I3D-P₁₅-I1F | 488/520 | 2727 | 2842 | 5536 | 1.0 | 1.9 |
| I3D-P₁₅-I2F | 488/520 | 2604 | 2446 | 6794 | 0.9 | 2.8 |
| I3D-P₁₅-I3F | 488/520 | 1934 | 2038 | 7097 | 1.1 | 3.5 |
| I3D-P₁₅-I4F | 488/520 | 2298 | 1894 | 7896 | 0.8 | 4.2 |
| I4D-P₁₅-I1F | 488/520 | 2342 | 2103 | 5885 | 0.9 | 2.8 |
| I4D-P₁₅-I2F | 488/520 | 1605 | 1449 | 6425 | 0.9 | 4.4 |
| I4D-P₁₅-I3F | 488/520 | 1905 | 2300 | 5977 | 1.2 | 2.6 |
| I4D-P₁₅-I4F | 488/520 | 3247 | 2908 | 9152 | 0.9 | 3.1 |

Abbreviations: As in TABLE 3 and E = glu(EDANS) (Example 6.2.4); C(F) = cysteine(fluorescein) (Example 6.2.6); C(AF) = cysteine(AlexaFluor 488) (Molecular Probes, Eugene OR; Example 6.2.6); Q = lys(QSY-7) (Example 6.2.8).
¹ΔF_{chelate} = F (+Ni⁺²)/F (−Ni⁺²).
²ΔF_{hybrid} = F (+target)/F (+Ni⁺²).

### Example 6.3.8

### Kinetics of Probe Binding to Target

Another important parameter in the practical application of probes is the rate at which the probes bind to their targets. Generally speaking, the greater the energy of the intramolecular chelate (the energy holding the probe in the closed conformation), the slower the rate of target binding is likely to be. Use of target-binding probes in real-time methods requires that this rate be fast enough to complete the method within a matter of minutes.

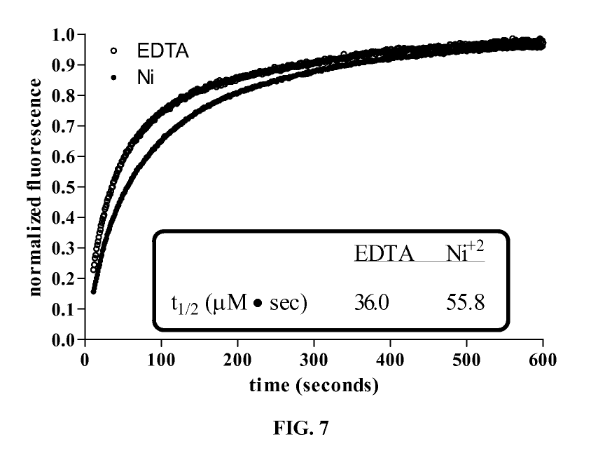

To determine the target binding rate, the fluorescence of probes labeled with a fluorophore and quencher were monitored over time after addition of the target. In this example, the target binding kinetics of the PNA probe prepared in Example 6.2.6 (I-D-P₁₅—C(F)-I) are followed by fluorescence. FIG. 7 illustrates the kinetic binding curves of this probe in the presence and absence of a transition metal. These data were fit to a first-order exponential association equation to determine the t_{1/2} of association as shown in the figure. As expected, in the presence of nickel, the association kinetics were notably slower than when EDTA is added to the sample instead of the metal. However, even with nickel the probe binding reached 50% completion in less than one minute, fast enough for use in real-time target detection methods.

In this experiment, the probe concentration was 100 nM and the target concentration was 1 μM, in a 10 mM potassium phosphate buffer, pH 7.2, with 10 mM NaCl. Data were collected using an SLM 8100 fluorometer by measuring the fluorescence of the sample containing only the probe (with or without metal), then adding the target DNA sequence and monitoring the change in fluorescence over time.

The present disclosure is not to be limited in scope by the specific embodiments disclosed in the examples which are intended as illustrations. Indeed, various modifications in addition to those shown and described herein will become apparent to those skilled in the art.

A number of references have been cited, the entire disclosures of which are incorporated herein by reference.

What is claimed is:

1. A probe, comprising a flexible binding element, a first and a second partial metal chelator and a transition metal, wherein the transition metal atom is shared between the first and the second partial metal chelator moieties, and wherein the first and the second partial metal chelators are attached to one another through a portion of the flexible binding element, and wherein a specificity factor of the probe comprises a value selected from the group consisting of K_{d}, IC₅₀, Kₘ, kₐ, k_{d}, log₂(PM/MM), Tₘ, T_{d-50} and T_{d-w}.

2. The probe according to claim 1, wherein the first and second partial metal chelators are independently selected from the group consisting of 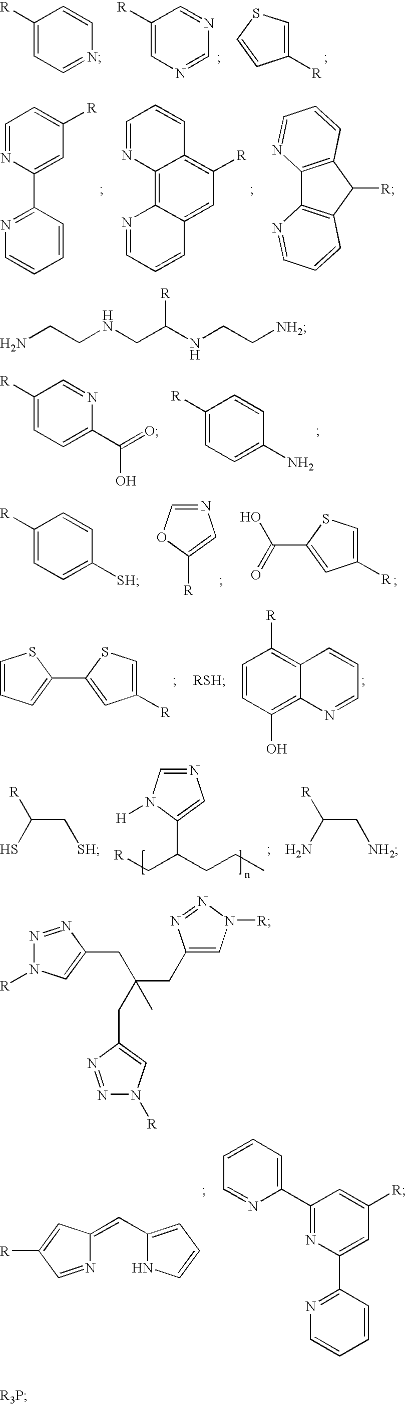 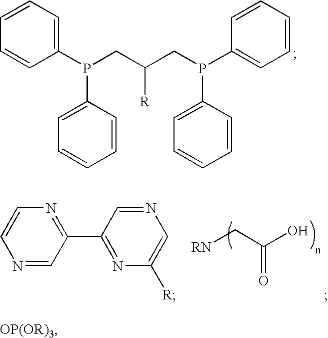
wherein R is the location of a covalent attachment to the probe.

3. The probe according to claim 1, further comprising a label, wherein the label is selected from the group consisting of fluorophores and quenchers, radioactive isotopes, antigenic determinants, non-radioactive isotopes, nucleic acids available for hybridization, altered fluorescence-polarization or altered light-scattering, chromogenic, chemiluminescent, electrochemically detectable, and combinations thereof.

4. The probe according to claim 1, wherein the flexible binding element comprises a segment selected from the group consisting of PNA, poly-morphilino, PNAMs, DNA, RNA, siRNA, peptide, oligosaccharide, and combinations thereof.

5. The probe according to claim 1, wherein the first and second partial metal chelators are the same.

6. The probe according to claim 1 wherein the transition metal is selected from the group consisting of zinc, cadmium, copper, nickel, ruthenium, platinum, palladium, cobalt, magnesium, barium, strontium, iron, vanadium, chromium, manganese, rhodium, silver, mercury, molybdenum, tungsten, calcium, lead, cerium, aluminum and thorium.

7. A coordination complex capable of disruption upon binding to a target, comprising a flexible binding element, a first and a second partial metal chelator and a transition metal, wherein the transition metal atom is shared between the first and the second partial metal chelator moieties, and wherein the first and the second partial metal chelators are attached to one another via a covalent bond through a portion of the flexible binding element, and wherein a specificity factor of the probe comprises a value selected from the group consisting of K_{d}, IC₅₀, Kₘ, kₐ, k_{d}, log₂(PM/MM), Tₘ, T_{d-50} and T_{d-w}.

8. The coordination complex according to claim 7, wherein the transition metal is selected from the group consisting of zinc, cadmium, copper, nickel, ruthenium, platinum, palladium, cobalt, magnesium, barium, strontium, iron, vanadium, chromium, manganese, rhodium, silver, mercury, molybdenum, tungsten, calcium, lead, cerium, aluminum and thorium.

9. The coordination complex of claim 7 wherein the first and second partial metal chelators are independently selected from the group consisting of 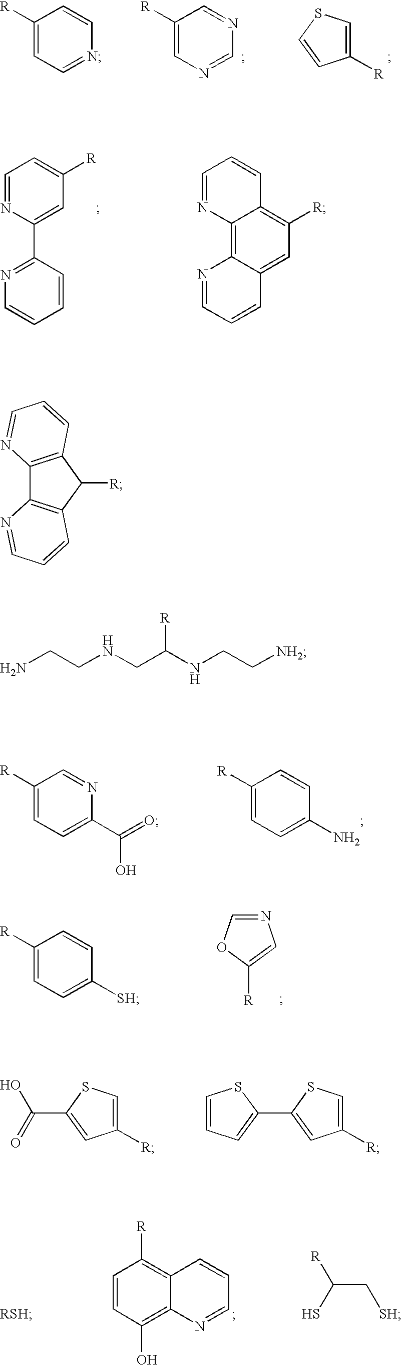 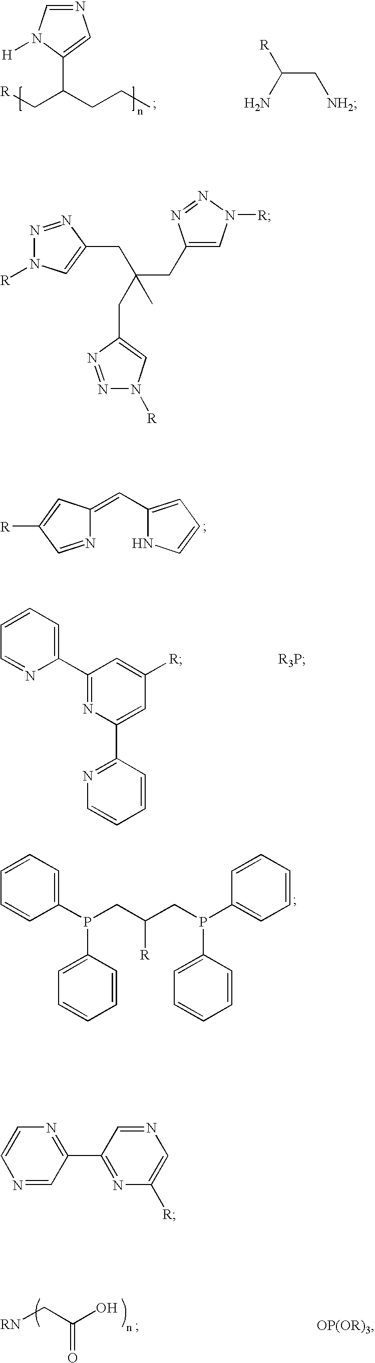
wherein R is the location of a covalent attachment to the probe.

10. A method for binding a target to a probe comprising:
a) providing a probe in a closed configuration, wherein the probe comprises a flexible binding element, a first and a second partial metal chelator and a transition metal, wherein the transition metal atom is shared between the first and the second partial metal chelator moieties, and wherein the first and the second partial metal chelators are attached to one another through a portion of the flexible binding element, and wherein a specificity factor of the probe comprises a value at least a great as the values selected from the group consisting of K_{d}, IC₅₀, Kₘ, kₐ, k_{d}, log₂(PM/MM), Tₘ, T_{d-50} and T_{d-w}; and
b) opening the closed configuration probe by contacting the probe with the target and the transition metal, wherein the target can only open the closed configuration probe with a high specificity factor.

11. The method according to claim 10, further comprising detecting the target bound to the probe.

12. The method according to claim 10, wherein the step of contacting occurs inside a cell.

13. The method according to claim 12, wherein the amount of an mRNA within the cell is reduced.

14. The method according to claim 12, wherein the amount of a protein within the cell is reduced.

15. The method according to claim 10 wherein the target is selected from the group consisting of DNA, RNA, mRNA, peptide, protein, oligosaccharide, and combinations thereof.

16. The method according to claim 10, wherein the probe is attached to a solid support.

17. The method according to claim 16, wherein the solid support is selected from the group consisting of a bead, glass slide, microarray, membrane, microtiter well and dipstick.

18. The method according to claim 10, wherein the transition metal is selected from the group consisting of zinc, cadmium, copper, nickel, ruthenium, platinum, palladium, cobalt, magnesium, barium, strontium, iron, vanadium, chromium, manganese, rhodium, silver, mercury, molybdenum, tungsten, calcium, lead, cerium, aluminum, thorium, and combinations thereof.

19. A method of claim 10, wherein the probe further comprises a label, wherein the label is selected from the group consisting of fluorophores and quenchers, radioactive isotopes, antigenic determinants, non-radioactive isotopes, nucleic acids available for hybridization, altered fluorescence-polarization or altered light-scattering, chromogenic, chemiluminescent, electrochemically detectable, and combinations thereof.

20. The method of claim 12, wherein the probe further comprises a spacer.

21. The method of claim 10, wherein the first and second partial metal chelators are independently selected from the group consisting of 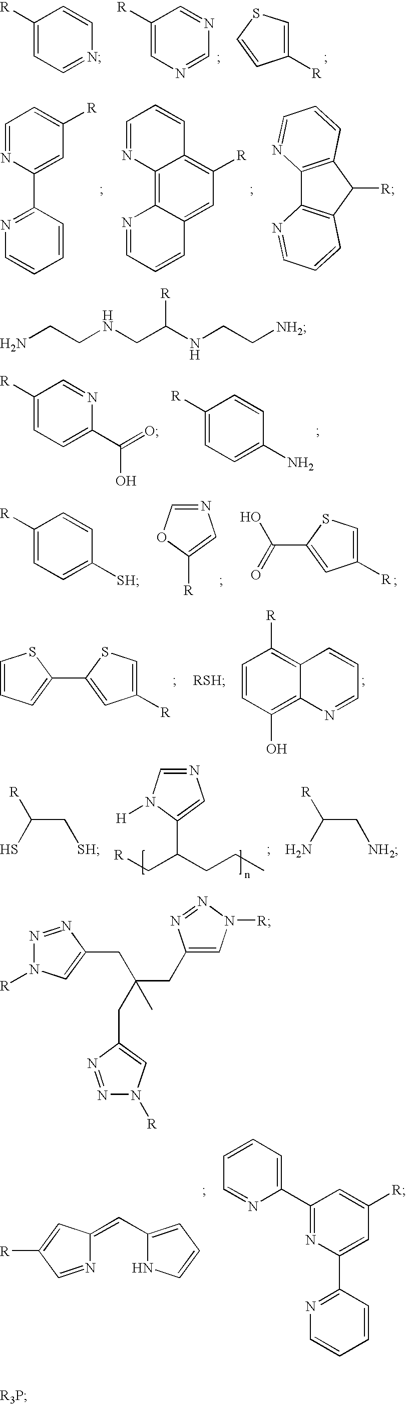 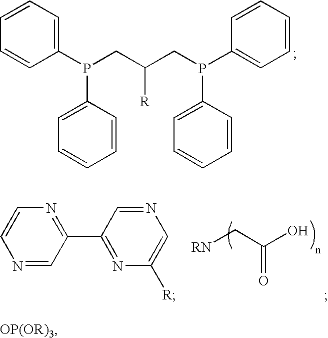
wherein R is an attachment to the probe.

22. The method of claim 10, wherein the portion of the flexible binding element is selected from the group consisting of PNA, poly-morphilino, PNAMS, DNA, RNA, siRNA, peptide, oligosaccharide, and combinations thereof.

23. The method of claim 10, wherein the first and second partial metal chelators are the same.

24. A method of binding a target comprising:
a) providing a probe comprising a first and a second partial metal chelator, wherein the first and second partial metal chelators are covalently attached to one another through a portion of a flexible binding element, wherein the partial metal chelators form a coordination complex with a transition metal, and wherein the coordination complex is disrupted when the binding element contacts a target; and
b) contacting the probe with the target and a transition metal.

25. A partial metal chelator synthon comprising formula (I) or formula (II): 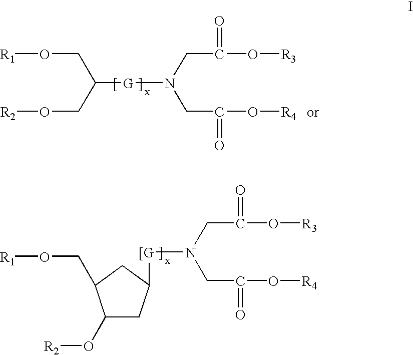
II
wherein
R₁ is a hydroxyl protecting group;
R₂ is selected from the group consisting of a linker; 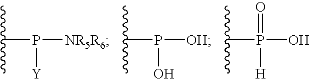
and salts thereof,
R₃ and R₄ are carboxyl protecting groups;
R₅ and R₆ are independently selected from the group consisting of C₃₋₁₀ branched alkyl and C₁₋₁₂ unbranched alkyl, and cyclic hydrocarbons;
Y is beta-cyanoethyl;
G is selected from the group consisting of alkyl, heteroalkyl, aryl, aryl(alkylene), heteroaryl, heteroaryl(alkylene), carbocycle, carbocyle(alkylene), heterocycle, heterocycle(alkylene), 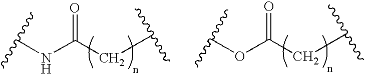
wherein n=1 to 10; and
X is from 0 to 10.

26. The partial metal chelator synthon according to claim 25, further comprising glass, wherein the glass is selected from the group consisting of controlled pore glass and flat glass.

27. The partial metal chelator synthon according to claim 26, wherein the partial metal chelator synthon consists of a compound having the structure

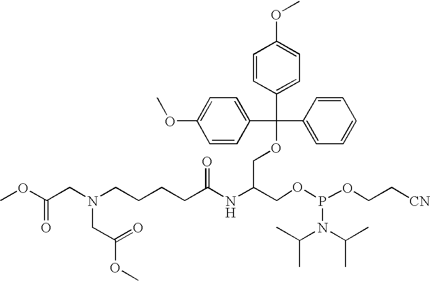

28. A microarray device comprising a solid phase substrate having a surface, wherein the solid phase surface comprises a plurality of known locations, and a plurality of probes, wherein each probe is bound to the substrate at a known location, wherein the probe comprises a flexible binding element, a first and a second partial metal chelator and a transition metal, wherein the transition metal atom is shared between the first and the second partial metal chelator moieties, and wherein the first and the second partial metal chelators are attached to one another through a portion of the flexible binding element, and wherein a specificity factor of the probe comprises a value selected from the group consisting of K_{d}, IC₅₀, Kₘ, kₐ, k_{d}, log₂(PM/MM), Tₘ, T_{d-50} and T_{d-w}.

29. The microarray device of claim 28 wherein the first and second partial metal chelators are independently selected from the group consisting of 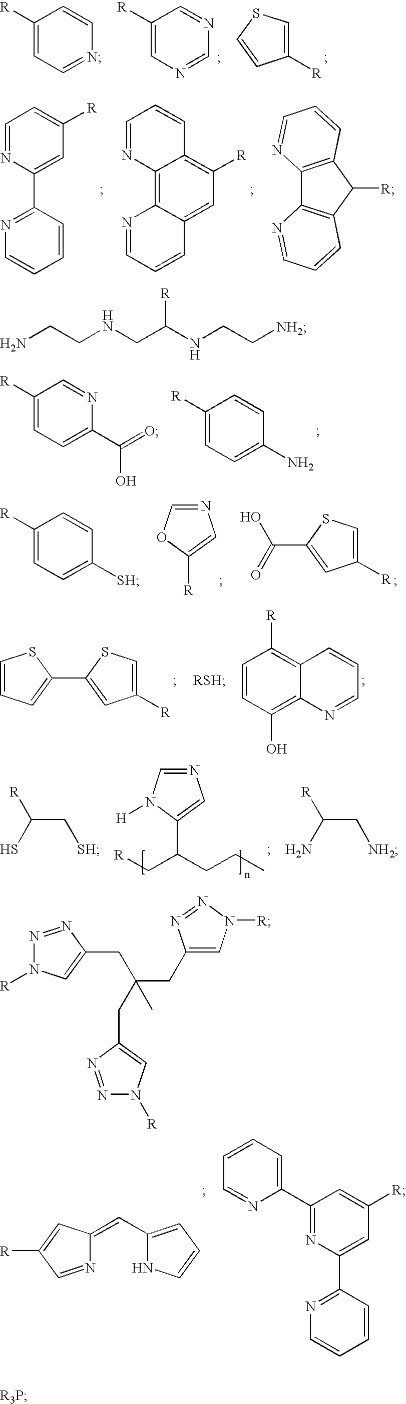 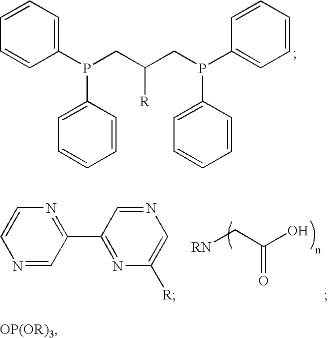
wherein R is the location of a covalent attachment to the probe.

30. The microarray device of claim 28 wherein the probe further comprises a label, wherein the label is selected from the group consisting of fluorophores and quenchers, radioactive isotopes, antigenic determinants, non-radioactive isotopes, nucleic acids available for hybridization, altered fluorescence-polarization or altered light-scattering, chromogenic, chemiluminescent, electrochemically detectable, and combinations thereof.

31. The microarray device of claim 28 wherein the flexible binding element comprises a segment selected from the group consisting of PNA, poly-morphilino, PNAMs, DNA, RNA, siRNA, peptide, oligosaccharide, and combinations thereof.

32. The microarray device of claim 28 wherein the transition metal is selected from the group consisting of zinc, cadmium, copper, nickel, ruthenium, platinum, palladium, cobalt, magnesium, barium, strontium, iron, vanadium, chromium, manganese, rhodium, silver, mercury, molybdenum, tungsten, calcium, lead, cerium, aluminum and thorium.
